(19) United States
(12) Patent Application Publication
(10) Pub. No.: US 2007/0142362 A1
(43) Pub. Date: Jun. 21, 2007

(54) 4,5-Dihydro-(1H)-pyrazole derivatives as cannabinoid CB1 receptor modulators

(75) Inventors: Josephus H.M. Lange, Weesp (NL); Wouter I. Iwema Bakker, Weesp (NL); Bernard J. Van Vliet, Weesp (NL); Martina A.W. Van Der Neut, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SOLVAY PHARMACEUTICALS B.V.

(21) Appl. No.: 11/641,725

(22) Filed: Dec. 20, 2006

Related U.S. Application Data
(60) Provisional application No. 60/751,667, filed on Dec. 20, 2005.

(30) Foreign Application Priority Data
Dec. 20, 2005 (EP) 05 112482.4

(51) Int. Cl. A61K 31/55; A61K 31/4709; A61K 31/4439; A61K 31/4152; C07D 403/02

(52) U.S. Cl. 514/217.09; 514/406; 548/374.1; 514/341; 514/310; 546/275.1; 546/148; 514/314; 546/169

(57) ABSTRACT

This invention is directed to 4,5-dihydro-(1H)-pyrazole(pyrazoline) derivatives as cannabinoid CB₁ receptor modulators, to pharmaceutical compositions containing these compounds, to methods for the preparation of these compounds, methods for preparing novel intermediates useful for their synthesis, and methods for preparing compositions. The invention also relates to the uses of such compounds and compositions, particularly their use in administering them to patients to achieve a therapeutic effect in disorders in which CB₁ receptors are involved, or that can be treated via manipulation of those receptors.
The compounds have the general formula (I) 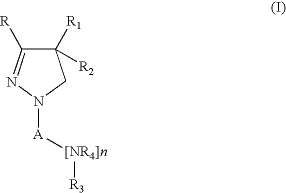 wherein the symbols have the meanings given in the specification.

### RELATED APPLICATIONS

This application claims priority benefit under Article 87 EPC of EP 065 112482.4 field on Dec. 20, 2005, and also under Title 35 §119(e) of U.S. Provisional Application No. 60/751,667 filed on Dec. 20, 2005, the contents of which are herein incorporated by reference.

4,5-DIHYDRO-(1H)-PYRAZOLE DERIVATIVES AS CANNABINOID
CB₁ RECEPTOR MODULATORS

| INDEX | page |
|---|---|
| Title of the invention | 1 |
| Index | 1 |
| Summary: technical field of the invention | 1 |
| Related applications | 2 |
| Background of the invention | 2 |
| Detailed description of the invention | 3 |
| Definitions of chemical terms | 10 |
| Definitions of other terms | 12 |
| Abbreviations | 15 |
| Examples | 17 |
| Example 1: Analytical methods | 17 |
| Example 2: General aspects of syntheses | 20 |
| Example 3: Synthesis and spectral data of intermediates | 26 |
| Example 4: Synthesis of specific compounds of the invention | 41 |
| Example 5: Formulations used in animal studies | 92 |
| Example 6: Pharmacological methods | 93 |
| Example 7: Pharmacological test results | 95 |
| Example 8: Pharmaceutical preparations | 96 |
| References | 99 |
| Claims | 101 |
| Abstract | 115 |

### SUMMARY

### TECHNICAL FIELD OF THE INVENTION

This invention is directed to 4,5-dihydro-(1H)-pyrazole(pyrazoline) derivatives as cannabinoid CB₁ receptor modulators, to pharmaceutical compositions containing these compounds, to methods for the preparation of these compounds, methods for preparing novel intermediates useful for their synthesis, and methods for preparing compositions. The invention also relates to the uses of such compounds and compositions, particularly their use in administering them to patients to achieve a therapeutic effect in disorders in which CB₁ receptors are involved, or that can be treated via manipulation of those receptors.

### BACKGROUND OF THE INVENTION

Cannabinoid receptors are part of the endo-cannabinoid system which is involved in several diseases, such as neurological, psychiatric, cardiovascular, gastrointestinal, reproductive, eating disorders and cancer (De Petrocellis, 2004; Di Marzo, 2004; Lambert and Fowler, 2005; Vandevoorde and Lambert, 2005).

CB₁ receptor modulators have several potential therapeutic applications such as medicaments for treating psychosis, anxiety, depression, attention deficits, memory disorders, cognitive disorders, appetite disorders, obesity, addiction, appetence, drug dependence, neurodegenerative disorders, dementia, dystonia, muscle spasticity, tremor, epilepsy, multiple sclerosis, traumatic brain injury, stroke, Parkinson's disease, Alzheimer's disease, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, as well as for the treatment of pain disorders, including neuropathic pain disorders, septic shock, glaucoma, diabetes, cancer, emesis, nausea, gastrointestinal disorders, gastric ulcers, diarrhoea, sexual disorders, impulse control disorders and cardiovascular disorders.

CB₂ receptors occur predominantly in the immune system (spleen, tonsils, immune cells), but also in astrocytes, microglial cells and in the brainstem and have been linked to the perception of neuropathic pain as well as allergy/asthma and (neuro)inflammatory conditions (Van Sickle, 2005).

Diarylpyrazoline derivatives having cannabinoid CB₁ receptor antagonistic or inverse agonistic affinity have been claimed in WO 01/70700, WO 03/026647, WO 03/026648, WO 2005/074920, and were described by Lange (2004, 2005).

Pyrazoline derivatives which act as agonists or partial agonists on the CB₁ receptor have not been reported yet, but certain pyrazoline derivatives have been claimed as vermin controlling agents (JP 61 189270).

There is abundant recent literature containing general information on CB receptor modulators (Lange and Kruse, 2004, 2005; Hertzog, 2004; Smith and Fathi, 2005; Thakur, 2005; Padgett, 2005; Muccioli, 2005; Raitio,2005; Muccioli and Lambert, 2006).

The objective of the present invention was to develop novel compounds with CB, receptor agonistic activity.

### DETAILED DESCRIPTION OF THE INVENTION

Surprisingly, we have found that the modification of the original 3-aryl or 3-heteroaryl group R in prior art pyrazolines of general formula (I) by a (substituted) alkyl moiety—in combination with a different substitution pattern at the 1-position of the pyrazoline moiety—results in novel compounds with potent CB₁ receptor affinity. Moreover, some of the compounds of the invention also have been found to act as partial agonists or full agonists at the CB₁ receptor, whereas other compounds of the invention have been found to act as antagonists or inverse agonists at the CB₁ receptor. The majority of the compounds of the invention showed also affinity for the CB₂ receptor. These compounds may act as CB₂ receptor agonists, CB₂ receptor antagonists or CB₂ receptor inverse agonists.

The present invention relates to compounds of the general formula (I): 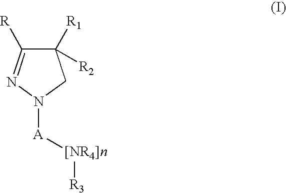
wherein
R represents a C₂₋₁₀ alkyl group, a C₄₋₁₀ alkenyl group, a C₄₋₁₀ alkynyl group, a C₂₋₁₀-heteroalkyl group, a C₅₋₈-cycloalkyl-C₁₋₅-alkyl group or a C₅₋₈-heterocycloalkyl-C₁₋₅-alkyl group wherein the heteroatom(s) are either N, O or S, which C₂₋₁₀ alkyl group, C₄₋₁₀ alkenyl group, C₄₋₁₀ alkynyl group, C₂₋₁₀-heteroalkyl group, C₅₋₈-cycloalkyl-C₁₋₅-alkyl group or C₅₋₈-heterocycloalkyl-C₁₋₅-alkyl group may be substituted with 1-5 substituents selected from methyl, ethyl, hydroxy, amino or fluoro, or R represents an aryl-C₁₋₃-alkyl group or an aryl-C₁₋₃-heteroalkyl group in which the aryl groups may be substituted with 1-5 substituents Y, which can be the same or different, selected from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethyl-sulfonyl, cyano, carbamoyl, sulfamoyl, phenyl and acetyl, or R represents a cyclopropyl group which cyclopropyl group may be substituted with 1-5 substituents selected from methyl, ethyl, fluoro or with a C₃₋₅ linear or branched alkyl group or with a benzyl or aryl group, in which the aryl or benzyl group may be substituted with 1-5 substituents Y,
R₁ represents hydrogen, hydroxy, C₁₋₃-alkoxy, acetyloxy or propionyloxy,
R₂ represents an aryl group which may be substituted with 1-5 substituents Y, wherein Y has the abovementioned meaning,
n is either 0 or 1
R₃ represents a linear C₃₋₁₀ alkyl group, a branched C₅₋₁₀ alkyl group, a cyclopropyl, cyclobutyl, cyclopentyl, cycloheptyl or cyclooctyl group, C₅₋₁₀ bicycloalkyl group, C₆₋₁₀ tricycloalkyl group or C₈₋₁₁ tetracycloalkyl group which groups may be substituted with 1-5 substituents selected from methyl, ethyl, hydroxy, amino, fluoro or R₃ represents a C₃₋₈ cycloalkyl group which C₃₋₈ cycloalkyl group is substituted with an aryl group which aryl group may be substituted with 1-5 substituents Y wherein Y has the abovementioned meaning, or R₃ represents a 2,2,2-trifluoroethyl or 2-fluoroethyl group or R₃ represents a cyclohexyl group which group is substituted with 1-5 substituents selected from methyl, ethyl, hydroxy, amino or fluoro, or R₃ represents a C₅₋₈ heterocycloalkyl group, C₆₋₁₀ bicycloheteroalkyl group, C₇₋₁₀ tricycloheteroalkyl group, which groups may be substituted with 1-5 substituents selected from methyl, ethyl, hydroxy, amino or fluoro, or R₃ represents a C₃₋₈ cycloalkyl-C₁₋₃-alkyl group, C₅₋₁₀-bicycloalkyl-C₁₋₃-alkyl group, C₆₋₁₀-tricycloalkyl-C₁₋₃-alkyl group, which groups may be substituted with 1-5 substituents selected from methyl, ethyl, hydroxy, amino or fluoro, or R₃ represents a branched or linear C₃₋₈ heterocycloalkyl-C₁₋₃-alkyl group, C₅₋₁₀ bicycloheteroalkyl-C₁₋₃-alkyl group, C₆₋₁₀ tricycloheteroalkyl-C₁₋₃-alkyl group, which groups may be substituted with 1-5 substituents selected from methyl, ethyl, hydroxy, amino or fluoro, or R₃ represents an aryl group, which group may be substituted with 1-5 substituents Y, wherein Y has the abovementioned meaning, or R₃ represents a aryl-C₁₋₅alkyl group or a diaryl-C₁₋₅alkyl group, in which groups the phenyl or heteroaromatic rings may be substituted with 1-5 substituents Y, wherein Y has the abovementioned meaning, or R₃ represents a linear or branched C₄₋₈ alkenyl or C₄₋₈ alkynyl group which linear or branched C₄₋₈ alkenyl or C₄₋₈ alkynyl group may be substituted with 1-3 fluoro atoms, or, when n=1, R₃ represents a branched or linear C₂₋₁₀ heteroalkyl group, containing 1-2 heteroatoms selected from N, O or S,
R₄ represents a hydrogen atom, a C₁₋₄ alkyl group or R₃ and R₄—together with the nitrogen atom to which they are bonded—form a saturated or unsaturated, non-aromatic or partly aromatic, monocyclic, bicyclic or tricyclic heterocyclic group having 5 to 11 ring atoms, which heterocyclic group may be substituted with 1-5 substituents selected from aryl, aryl-C₁₋₃-alkyl, diarylmethyl, or Y, wherein Y has the abovementioned meaning
A represents a carbonyl (C═O), thiocarbonyl (C═S) or sulfonyl (SO₂) group with the proviso that when A represents a thiocarbonyl (C═S), group, n has the value 1,
   and stereoisomers, prodrugs and N-oxides thereof, and isotopically-labelled compounds of formula (I), as well as pharmacologically acceptable salts, hydrates, solvates, complexes and conjugates of said compounds of formula (I) and its stereoisomers, prodrugs, N-oxides, or isotopically-labelled analogs.

The invention particularly relates to compounds of the general formula (I) wherein R₁ represents a hydrogen atom, and the other symbols have the meanings as given above.

More particular, the invention relates to compounds of the general formula (I) wherein R₁ represents a hydrogen atom, A represents a carbonyl group, and the other symbols have the meanings as given above.

Even more particular, the invention relates to compounds of the general formula (I) wherein R₁ represents a hydrogen atom, A represents a carbonyl group, R₂ represents a phenyl, thienyl or pyridyl group, which phenyl, pyridyl or thienyl group may be substituted with 1, 2 or 3 substituents Y, and the other symbols have the meanings as given above.

Also in particular, the invention relates to compounds of the general formula (I) wherein n=1, R₁ represents a hydrogen atom, A represents a carbonyl group, R₂ represents a phenyl, thienyl or pyridyl group, which phenyl, pyridyl or thienyl group may be substituted with 1, 2 or 3 substituents Y, and the other symbols have the meanings as given above.

Likewise, the invention particularly relates to compounds of the general formula (I) wherein n=1, R₁ and R₄ represent hydrogen atoms, A represents a carbonyl group, R₂ represents a phenyl, thienyl or pyridyl group, which phenyl, pyridyl or thienyl group may be substituted with 1, 2 or 3 substituents Y, and the other symbols have the meanings as given above.

Most particularly the invention relates to compounds of the general formula (I) wherein n=1, R represents a C₃₋₈ branched or linear alkyl group, which C₃₋₈ branched or linear alkyl group may be substituted with 1-3 fluoro atoms, R₁ and R₄ represent hydrogen atoms, R₂ represents a phenyl or pyridyl group, which phenyl or pyridyl group may be substituted with 1, 2 or 3 substituents Y, and the other symbols have the meanings as given above.

The compounds of the invention of the general formula (I), as well as the pharmacologically acceptable salts thereof, have cannabinoid CB, receptor modulating activity. They are useful in the treatment of disorders in which cannabinoid receptors are involved, or that can be treated via manipulation of those receptors.

The invention is also directed to:
a pharmaceutical composition for treating, for example, a disorder or condition that may be treated by modulating cannabinoid CB₁ receptors, the composition comprising a compound of formula (I) or a pharmaceutically acceptable salt thereof, and a pharmaceutically acceptable carrier;
a method of treatment of a disorder or condition that may be treated by modulating cannabinoid CB₁ receptors, the method comprising administering to a mammal in need of such treatment a compound of formula (I) or a pharmaceutically acceptable salt thereof;
a pharmaceutical composition for treating, for example, a disorder or condition selected from the group consisting of psychosis, anxiety, depression, attention deficits, memory disorders, cognitive disorders, appetite disorders, obesity, addiction, appetence, drug dependence, neurodegenerative disorders, dementia, dystonia, muscle spasticity, tremor, multiple sclerosis, traumatic brain injury, stroke, Parkinson's disease, Alzheimer's disease, epilepsy, Huntington's disease, Tourette's syndrome, cerebral ischaemia, cerebral apoplexy, craniocerebral trauma, stroke, spinal cord injury, neuroinflammatory disorders, plaque sclerosis, viral encephalitis, demyelinisation related disorders, as well as for the treatment of pain disorders, including neuropathic pain disorders, septic shock, glaucoma, diabetes, cancer, emesis, nausea, gastrointestinal disorders, gastric ulcers, diarrhoea, sexual disorders, impulse control disorders and cardiovascular disorders;
a method of treatment of a disorder or condition selected from the group consisting of the disorders listed herein, the method comprising administering to a mammal in need of such treatment a compound of formula (I) or a pharmaceutically acceptable salt thereof;
a pharmaceutical composition for treatment of a disorder or condition selected from the group consisting of the disorders listed herein, the composition comprising a compound of formula (I) or a pharmaceutically acceptable salt thereof, and a pharmaceutically acceptable carrier;
a method of treatment of a disorder or condition that may be treated by modulating cannabinoid CB₁ receptors, the method comprising administering to a patient in need of such treatment a compound of formula (I) or a pharmaceutically acceptable salt thereof.
a method of antagonizing a cannabinoid CB, receptor, which comprises administering to a subject in need thereof, an effective amount of a compound of formula (I);

The invention also provides the use of a compound or salt according to formula (I) for the manufacture of a medicament.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed. Such other therapeutic agent(s) may be administered prior to, simultaneously with, or following the administration of the compounds of the invention.

The invention also provides compounds, pharmaceutical compositions, kits and methods for the treatment of a disorder or condition that may be treated by modulating cannabinoid CB₁ receptors, the method comprising administering to a patient in need of such treatment a compound of formula (I) or a pharmaceutically acceptable salt thereof.

The compounds of the invention possess cannabinoid CB₁ receptor modulating activity. The (ant)agonizing activities of the compounds of the invention is readily demonstrated, for example, using one or more of the assays described herein or known in the art.

The invention also provides methods of preparing the compounds of the invention and the intermediates used in those methods.

The compounds of the present invention may contain one or more asymmetric centers and can thus occur as racemates and racemic mixtures, single enantiomers, diastereomeric mixtures and individual diastereomers.

All compounds of the present invention do contain at least one chiral centre (at the 4-position of the 4,5-dihydropyrazole ring). Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. The independent syntheses of these diastereomers or their chromatographic separations may be achieved as known in the art by appropriate modification of the methodology disclosed herein. Their absolute stereochemistry may be determined by X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base, such as for example (−)di-p-toluoyl-D-tartaric acid and/or (+)-di-p-toluoyl-L-tartaric acid The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

Cis and trans isomers of the compound of formula (I) or a pharmaceutically acceptable salt thereof are also within the scope of the invention, and this also applies to tautomers of the compounds of formula (I) or a pharmaceutically acceptable salt thereof.

Some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Isotopically-labeled compound of formula (I) or pharmaceutically acceptable salts thereof, including compounds of formula (I) isotopically-labeled to be detectable by PET or SPECT, are also included within the scope of the invention, and same applies to compounds of formula (I) labeled with [¹³C]—, [¹⁴C]—, [³H]—, [¹⁸F]—, [¹²⁵I]— or other isotopically enriched atoms, suitable for receptor binding or metabolism studies.

Definitions of Chemical Terms

The term ‘alkyl’ refers to straight or branched saturated hydrocarbon radicals. ‘Alkyl(C₁₋₃)’ for example, means methyl, ethyl, n-propyl or isopropyl, and ‘alkyl(C₁₋₄)’ means ‘methyl, ethyl, n-propyl, isopropyl, n-butyl, sec-butyl, isobutyl or tert-butyl’. The term ‘alkenyl’ denotes straight or branched hydrocarbon radicals having one or more carbon-carbon double bonds, such as vinyl, allyl, butenyl, etc.. In ‘alkynyl’ groups the straight or branched hydrocarbon radicals have one or more carbon-carbon triple bonds, such as ethynyl, propargyl, 1-butynyl, 2-butynyl, etc.. The term ‘acyl’ means alkyl(C₁₋₃) carbonyl, arylcarbonyl or aryl-alkyl(C₁₋₃)carbonyl. ‘Hetero’ as in ‘heteroalkyl, heteroaromatic’ etc. means either N, O or S. ‘heteroalkyl’ includes alkyl groups with heteroatoms in any position, thus including N-bound, O-bound or S-bound alkyl groups. The abbreviation ‘aryl’ means monocyclic or fused bicyclic aromatic or heteroaromatic groups, which heteroaromatic groups contain one or two heteroatoms selected from the group (N, O, S). Aryl groups include but are not limited to furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, phenyl, indazolyl, indolyl, indolizinyl, isoindolyl, benzo[b]furanyl, 1,2,3,4-tetrahydronaphtyl, 1,2,3,4-tetrahydroisoquinolinyl, indanyl, indenyl, benzo[b]thiophenyl, 2,3-dihydro-1,4-benzodioxin-5-yl, benzimidazolyl, benzothiazolyl, quinolinyl, isoquinolinyl, phtalazinyl, quinazolinyl, quinoxalinyl, 1,8-naphthyridinyl, naphthyl. The abbreviation ‘halogen’ means chloro, fluoro, bromo or iodo. The abbreviation ‘C₃₋₈-cycloalkyl’ means cyclopropyl, cyclobutyl, cyclopentyl, cyclohexyl, cyclopheptyl or cyclooctyl. The abbreviation ‘C₅₋₈ heterocycloalkyl’ refers to (N, O, S) heteroatom containing rings including but not limited to piperidinyl, morpholinyl, azepanyl, pyrrolidinyl, thiomorpholinyl, piperazinyl, tetrahydrofuryl, tetrahydropyranyl. The abbreviation ‘C₅₋₁₀ bicycloalkyl group’ refers to carbo-bicyclic ring systems including but not limited to bicyclo[2.2.1]heptanyl, bicyclo[3.3.0]octanyl or the bicyclo[3.1.1]heptanyl group. The abbreviation ‘C₆₋₁₀ tricycloalkyl group’ refers to carbo-tricyclic ring systems including but not limited to the 1-adamantyl, noradamantyl or the 2-adamantyl group. The abbreviation ‘C₈₋₁₁ tetracycloalkyl group’ refers to carbo-tetracyclic ring systems including but not limited to the cubyl, homocubyl or bishomocubyl group.

The terms “oxy”, “thio” and “carbo” as used herein as part of another group respectively refer to an oxygen atom, a sulphur atom and a carbonyl (C═O) group, serving as linker between two groups, such as for instance hydroxyl, oxyalkyl, thioalkyl, carboxyalkyl, etc. The term “amino” as used herein alone or as part of another group refers to a nitrogen atom that may be either terminal or a linker between two other groups, wherein the group may be a primary, secondary or tertiary (two hydrogen atoms bonded to the nitrogen atom, one hydrogen atom bonded to the nitrogen atom and no hydrogen atoms bonded to the nitrogen atom, respectively) amine. The terms “sulfinyl” and “sulfonyl” as used herein as part of another group respectively refer to an —SO— or an —SO₂— group.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

N-oxides of the compounds mentioned above are in the scope of the present invention. Tertiary amines may or may not give rise to N-oxide metabolites. The extent to what N-oxidation takes place varies from trace amounts to a near quantitative conversion. N-oxides may be more active than their corresponding tertiary amines or less active. Whilst N-oxides are easily reduced to their corresponding tertiary amines by chemical means, in the human body this happens to varying degrees. Some N-oxides undergo nearly quantitative reductive conversion to the corresponding tertiary amines, in other cases the conversion is a mere trace reaction or even completely absent (Bickel, 1969).

Definitions of Other Terms

With reference to substituents, the term “independently” means that when more than one of such substituents is possible, such substituents may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Any compound that can be converted in vivo to provide the bioactive agent (i.e., the compound of formula (I)) is a prodrug within the scope and spirit of the application. Prodrugs are therapeutic agents which are inactive per se but are transformed into one or more active metabolites. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Prodrugs are bioreversible derivatives of drug molecules used to overcome some barriers to the utility of the parent drug molecule. These barriers include, but are not limited to, solubility, permeability, stability, presystemic metabolism and targeting limitations (Bundgaard, 1985; King, 1994; Stella, 2004; Ettmayer, 2004; Jarvinen, 2005). Prodrugs, i.e. compounds which when administered to humans by any known route, are metabolised to compounds having formula (I), belong to the invention. In particular this relates to compounds with primary or secondary amino or hydroxy groups. Such compounds can be reacted with organic acids to yield compounds having formula (I) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Dose. The affinity of the compounds of the invention for cannabinoid CB₁ receptors was determined as described below. From the binding affinity measured for a given compound of formula (I), one can estimate a theoretical lowest effective dose. At a concentration of the compound equal to twice the measured Kᵢ-value, nearly 100% of the cannabinoid CB₁ receptors likely will be occupied by the compound. Converting that concentration to mg of compound per kg of patient yields a theoretical lowest effective dose, assuming ideal bioavailability. Pharmacokinetic, pharmaco-dynamic, and other considerations may alter the dose actually administered to a higher or lower value. The dose of the compound to be administered will depend on the relevant indication, the age, weight and sex of the patient and may be determined by a physician. The dosage will preferably be in the range of from 0.01 mg/kg to 10 mg/kg. The typical daily dose of the active ingredients varies within a wide range and will depend on various factors such as the relevant indication, the route of administration, the age, weight and sex of the patient and may be determined by a physician. In general, oral and parenteral dosages will be in the range of 0.1 to 1,000 mg per day of total active ingredients.

The term “therapeutically effective amount” as used herein refers to an amount of a therapeutic agent to treat or prevent a condition treatable by administration of a composition of the application. That amount is the amount sufficient to exhibit a detectable therapeutic, preventative or ameliorative response in a tissue system, animal or human. The effect may include, for example, treatment or prevention of the conditions listed herein. The precise effective amount for a subject will depend upon the subject's size and health, the nature and extent of the condition being treated, recommendations of the treating physician (researcher, veterinarian, medical doctor or other clinician), and the therapeutics or combination of therapeutics selected for administration. Thus, it is not useful to specify an exact effective amount in advance.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention, or separately by reacting compounds of the invention with pharmaceutically acceptable non-toxic bases or acids, including inorganic or organic bases and inorganic or organic acids.

The term “treatment” as used herein refers to any treatment of a mammalian, preferably human condition or disease, and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

The term ‘medical therapy’ as used herein is intended to include prophylactic, diagnostic and therapeutic regimens carried out in vivo or ex vivo on humans or other mammals.

The term “subject” as used herein, refers to an animal, preferably a mammal, most preferably a human, who has been the object of treatment, observation or experiment.

### ABBREVIATIONS

ACN acetonitrile
API-ES atmospheric pressure ionization—electron spray
BOC tert-butoxycarbonyl
BSA bovine serum albumin
CB₁ cannabinoid receptor subtype-1
CB₂ cannabinoid receptor subtype-2
CHO Chinese Hamster Ovary (cells)
CNS central nervous system
CUR curtain gas
DF deflector voltage
DIPEA N,N-diisopropylethylamine
DMAP 4-dimethylaminopyridin
DMEM Dulbecco's Modified Eagle's Medium
DMSO dimethylsulfoxide
DSC differential scanning calorimetry
EDCl 1-(3-dimethylaminopropyl)-3-ethylcarbodiimide hydrochloride
EP entrance potential
FP focusing potential
g gram(s)
h hour(s)
HOBt N-hydroxybenzotriazole
HPLC high performance liquid chromatography
IBMX 3-isobutyl-1-methylxanthine
IS ionspray voltage
MeOH methanol
mg milligram(s)
min minute(s)
ml milliliter(s)
m.p. melting point c.q. melting range
MTBE methyl tert-butylether
NEB nebulizer gas
NMM N-methylmorpholine
PBS phosphate buffered saline
PET positron emission tomography
R_{f} retention factor (thin layer chromatography)
Rₜ retention time (LC/MS)
RT room temperature
SPECT single photon emission computed tomography
TEM temperature
THF tetrahydrofuran

### EXAMPLES

### Example 1

### Materials and Methods

¹H NMR spectra were recorded on either a Varian 300 MHz instrument, a Varian UN400 instrument (400 MHz) using DMSO-d₆ or CDCl₃ as solvents with tetramethylsilane as an internal standard. ¹³C NMR spectra were recorded on a Varian UN400 instrument using CDCl₃ as solvent. Chemical shifts are given in ppm (δ scale) downfield from tetramethylsilane. Coupling constants (J) are expressed in Hz. Flash chromatography was performed using silica gel 60 (0.040-0.063 mm, Merck). Column chromatography was performed using silica gel 60 (0.063-0.200 mm, Merck). Sepacore chromatographic separations were carried out using Supelco equipment, VersaFLASH™ columns, VersaPak™ silica cartridges, Büchi UV monitor C-630, Büchi Pump module C-605, Büchi fraction collector C-660 and Buchi pump manager C-615. Melting points were recorded on a Büchi B-545 melting point apparatus or determined by DSC (differential scanning calorimetry) methods. Optical rotations ([α]_{D}) were measured on an Optical Activity polarimeter. Specific rotations are given as deg/dm, the concentration values are reported as g/100 mL of the specified solvent and were recorded at 23° C.

LC-MS instrumentation for method A and method B: Hardware: An Agilent 1100 LC/MS system was used consisting of:

| | |
|---|---|
| G1322A | solvent degasser |
| G1311A | quaternary pump |
| G1313A | auto sampler |
| G1316A | column oven + switch |
| G1315B | DAD + standard flow cell |
| G1946D | (SL)-MSD |

Method A:
Column: Discovery C₁₈ (150×4.6 mm) Supelco
Mobile phase: 100% Solution B (16 min)
Flow rate: 1.0 ml/min.
UV wavelength: 216 & 251 nm
Sample: ˜1 mg/ml in MeOH
Injected volume: 3 μl
Temperature: 22° C.
Mass detection.: API-ES positive
Solution B: 9.65 g Ammoniumacetate; 250 ml H₂O; 1350 ml MeOH; 900 ml Acetonitrile
   Method B:
Column: Agilent Zorbax Extend-C18 (4.6*50 mm; 3.5 μm)
Mobile phase: Gradient: 0-3 minutes: Solution A/Solution B=20/80 (v/v)).>3 minutes: Solution B, unless indicated otherwise.
Flow rate: 1.0 ml/min.
UV wavelength: 218 and 250 nm
Sample: ˜1 mg/ml in MeOH
Injected volume: 1.0μl
Temperature: 22° C.
Mass detection: API-ES positive & negative
Solution A: 9.65 g Ammoniumacetate; 2250 ml H₂O; 150 ml MeOH; 100 ml Acetonitrile
Solution B: 9.65 g Ammoniumacetate; 250 ml H₂O; 1350 ml MeOH; 900 ml Acetonitrile
   Preparative LC/MS Instrumentation and Procedure for Method C
Sciex API 150 EX masspectrometer with electron spray,
2 Shimadzu LC8A LC pump,
Shimadzu SCL-10A VP system controller,
Shimadzu SPD-10A VP UV meter,
Gilson 215 injector/collector,

Column: Phenomenex Luna C18 (2) :150×21.2×5 μ

Eluant: A 100% Water+0.1% Formic acid on pH=3 : B 100% Acetonitrile+0.1% Formic acid

Injection: 2.5 ml

Splitter: 1 to 50,000 with a make-up flow of 0.2 ml/min (25% H₂O/75% ACN met 0.25% HCOOH)

MS scan from: 100-900 amu step 1 amu scan time 1 sec.

Method: Flow rates and gradient profiles.

| Total Time (min) | Flow rate (ml/min) | A % (v/v) | B % (v/v) |
|---|---|---|---|
| 0 | 5 | 95 | 5 |
| 2 | 5 | 95 | 5 |
| 2.1 | 20 | 95 | 5 |
| 12 | 20 | 0 | 100 |
| 14 | 20 | 0 | 100 |
| 14.5 | 20 | 95 | 5 |
| 15 | 20 | 95 | 5 |

Preparative LC/MS Instrumentation and Procedure for Method D
Analytical 3 Minutes Method

The LC-MS system consists of 2 Perkin-Elmer series 200 micro pumps. The pumps are connected to each other by a 50 ul tee mixer. The mixer is connected to the Gilson 215 auto sampler.

The LC method is:

| step | total time | flow (ul/min) | A (%) | B (%) |
|---|---|---|---|---|
| 0 | 0 | 2300 | 95 | 5 |
| 1 | 1.8 | 2300 | 0 | 100 |
| 2 | 2.5 | 2300 | 0 | 100 |
| 3 | 2.7 | 2300 | 95 | 5 |
| 4 | 3.0 | 2300 | 95 | 5 |

A=100% Water with 0.2% HCOOH and 10 mmol NH4COOH pH=±3
B=100% ACN with 0.2% HCOOH

The auto sampler has a 2 ul injection loop. The auto sampler is connected to a Waters Atlantis C18 30*4.6 mm column with 3 um particles. The column is thermo stated in a Perkin-Elmer series 200 column oven at 40 degrees Celsius. The column is connected to an Applied biosystems ABI 785 UV meter with a 2.7 ul flow cel. The wavelength is set to 254 nm. The UV meter is connected to a Sciex API 150EX mass spectrometer. The mass spectrometer has the following parameters:
Scan range: 150-900 Amu
Polarity: positive
Scan mode: profile
Resolution Q1: UNIT
Step size: 0.10 amu
Time per scan: 0.500 sec
NEB: 10
CUR: 10
IS: 5200
TEM: 325
DF: 30
FP: 225
EP: 10

The light scattering detector is connected to the Sciex API 150. The light scattering detector is a Polymerlabs PLS2100 operating at 70° C. and 1.7 bar N₂ pressure. The complete systems is controlled by a Dell precision 370 computer operating under Windows 2000.

### Example 2

### General Aspects of Syntheses

Pyrazoline derivatives can be obtained by published methods (Barluenga, 1999 (and references cited therein); Wang, 2003). The synthesis of compounds having formula (I) is outlined in Scheme 1. Ketone derivatives of general formula (II) can be made by various methods known to those skilled in the art. Examples are the application of a so-called Weinreb amide RC(═O)N(OCH₃)CH₃ which can be reacted with a Grignard reagent R₂CH₂MgCl or R₂CH₂MgBr or a reaction of RMgBr or RMgCl with a Weinreb amide of general formula R₂CH₂C(═O)N(OCH₃)CH₃. Alternatively, a Grignard reagent R₂CH₂MgCl or R₂CH₂MgBr can be reacted with a cyanide analog R₁CN, followed by acidic hydrolysis, for example by using hydrochloric acid. A ketone derivative of general formula (II) wherein R and R₂ have the abovementioned meaning can be reacted with formaldehyde in the presence of an amine, such as piperidine and an acid, for example acetic acid, in an inert organic solvent such as methanol to give a compound of general formula (III), wherein R and R₂ have the abovementioned meaning. This reaction can be classified as a so-called Mannich reaction, followed by elimination of the applied amine. Alternatively, a ketone derivative of general formula (II) wherein R and R₂ have the abovementioned meaning can be reacted with N,N,N′,N′-tetramethyldiaminomethane in acetic anhydride to give a compound of general formula (III), wherein R and R₂ have the abovementioned meaning (Ogata, 1987^{a}, 1987^{b}). The compound of general formula (III) can be reacted with hydrazine or hydrazine hydrate in the presence of an inert organic solvent such as ethanol to give a pyrazoline derivative of general formula (IV), wherein R and R₂ have the abovementioned meaning and R₁ represents a hydrogen atom. Alternatively, the compound of general formula (III) can be oxidized with an oxidizing reagent such as hydrogen peroxide to give a epoxyketone derivative of general formula (V), wherein R and R₂ have the abovementioned meaning. A compound of general formula (V) can be reacted with hydrazine or hydrazine hydrate in the presence of an inert organic solvent such as ethanol to give a pyrazoline derivative of general formula (IV), wherein R and R₂ have the abovementioned meaning and R₁ represents a hydroxy group.

A compound of general formula (IV) can be reacted with a carboxylic acid R₃—CO₂H wherein R₃ has the abovementioned meaning in the presence of an so-called activating reagent or coupling reagent in an inert organic solvent such as dichloromethane to give a pyrazoline derivative of general formula (I), wherein n=0, A represents a carbonyl group and all other symbols have the meanings as given above. Additional information on activating and coupling methods of amines to carboxylic acids can be found in the literature (Bodanszky and Bodanszky, 1994; Akaji, 1994; Albericio, 1997; Montalbetti and Falque, 2005).

Alternatively, a compound of general formula (IV) wherein R, R₁ and R₂ have the abovementioned meaning can be reacted with an acid chloride R₃—COCl wherein R₃ has the abovementioned meaning to give a pyrazoline derivative of general formula (I), wherein n=0, A represents a carbonyl group and all other symbols have the meanings as given above.

A compound of general formula (IV) wherein R, R₁ and R₂ have the abovementioned meaning can be reacted with an isocyanate derivative R₃—N═C═O (VII) wherein R₃ has the abovementioned meaning in the presence of an inert organic solvent such as diethyl ether to give a pyrazoline-1-carboxamide derivative of general formula (I), wherein n=1 and R₄ represents H, A represents a carbonyl group and all other symbols have the meanings as given above. Isocyanates R₃—N═C═O can also be prepared in situ from the corresponding amine R₃—NH₂ and a so-called carbonyl donor such as phosgene, diphosgene (trichloromethyl chloroformate) or triphosgene (bis(trichloromethyl) carbonate). Alternatively, isocyanates R₃—N═C═O can be prepared from the corresponding carboxylic acid R₃—COOH via the acylazide R₃—CON₃ in a so-called Curtius rearrangement.

An amine of general formula R₃R₄NH wherein R₃ and R₄ have the abovementioned meaning can be reacted with a carbonylating agent such as phosgene and the like in the presence of an inert organic solvent such as toluene or benzene to give a compound of general formula (VI), wherein L represents a so-called leaving group such as chloride. A compound of general formula (VI) wherein L represents a so-called leaving group can be reacted with a compound of general formula (IV) wherein R, R₁ and R₂ have the abovementioned meaning to give a pyrazoline derivative of general formula (I), wherein n=1 and all other symbols have the meanings as given above. Preferably, a base such as triethylamine or Hunigs base may be added in such reactions. Furthermore, 4-(dimethylamino)pyridine (DMAP) may serve as a catalyst in such reactions.

A compound of general formula (IV) wherein R, R₁ and R₂ have the abovementioned meaning can be reacted with an isothiocyanate derivative R₃—N═C═S (VIIa) wherein R₃ has the abovementioned meaning in the presence of an inert organic solvent such as tetrahydrofuran to give a pyrazoline-1-carbothioamide derivative of general formula (I), wherein n=1 and R₄ represents H, A represents a thiocarbonyl group and all other symbols have the meanings as given above.

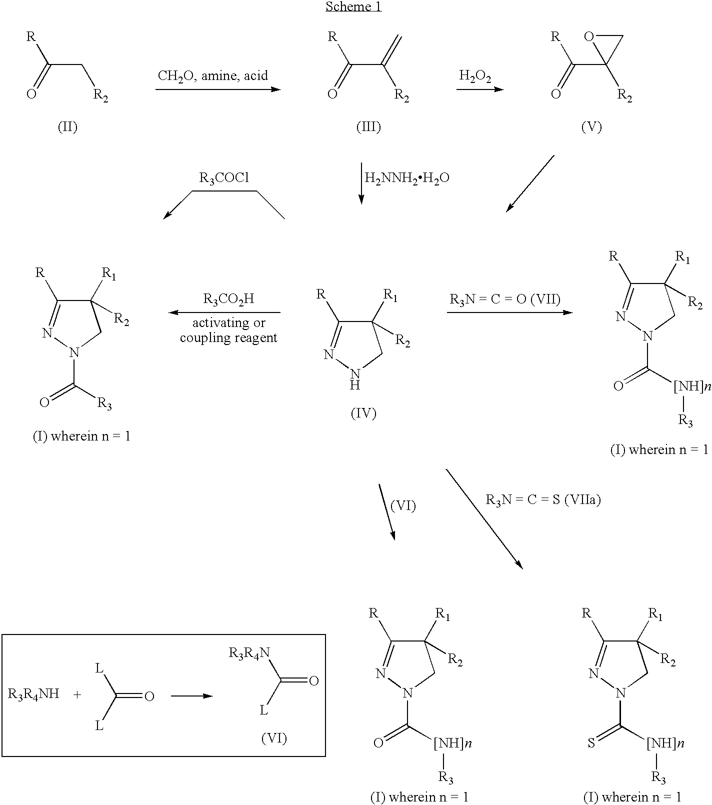

Alternatively, a compound of general formula (IV) wherein R and R₂ have the abovementioned meaning and R₁ represents a hydrogen atom can be reacted with phosgene, diphosgene or triphosgene to give a compound of general formula (VIII) wherein R and R₂ have the abovementioned meaning and R₁ represents a hydrogen atom (Scheme 2). A compound of general formula (VIII) can be reacted with a compound R₃R₄NH to give a pyrazoline-1-carboxamide derivative of general formula (I), wherein n=1, A represents a carbonyl group. A compound of general formula (IV) wherein R and R₂ have the abovementioned meaning and R₁ represents a hydrogen atom can be reacted with a sulfonylchloride derivative of general formula R₃SO₂Cl to give a pyrazoline derivative of general formula (I), wherein n=0, A represents a sulfonyl group and all other symbols have the meanings as given above. Preferably, a base such as triethylamine or Hunigs base (DIPEA) may be added in such reactions.

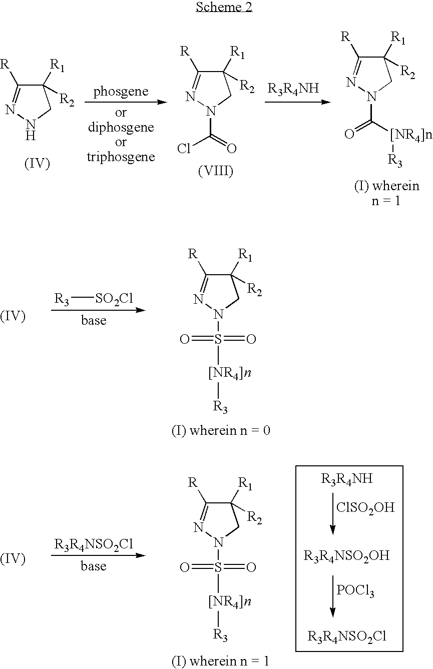

A compound of general formula (IV) wherein R and R₂ have the abovementioned meaning and R₁ represents a hydrogen atom can be reacted with a compound of general formula R₃R₄NSO₂Cl to give a pyrazoline derivative of general formula (I), wherein n=1, A represents a sulfonyl group and all other symbols have the meanings as given above. Preferably, a base such as triethylamine or Hünigs base (DIPEA) may be added in such reactions.

A compound of general formula R₃R4NSO₂Cl can be obtained from a reaction of a sulfamic acid derivative R₃R₄NSO₂OH with a chlorinating agent such as POCl₃ in an inert organic solvent such as dichloromethane. A compound of general formula R₃R₄NSO₂OH can be obtained from a reaction of an amine R₃R₄NH and chlorosulfonic acid in an inert organic solvent such as dichloromethane. Preferably, a base such as triethylamine or Hünigs base (DIPEA) may be added in such a reaction.

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Compounds of the general formula (III), wherein R represent a phenyl group which is substituted with 1-3 substituents Y1 wherein Y1 represents halogen, CF₃, OCF₃ or OCH₃, or R represents a pyridyl or thienyl group, and R₂ represents a n-butyl, n-propyl, 1,1-dimethylpropyl, 1,1-dimethylbutyl, 3,3,3-trifluoropropyl, 4,4,4-trifluorobutyl or 1,1-dimethyl-3,3,3-trifluoropropyl group, or R represent a phenyl group and R₂ represents a 1,1-dimethylpropyl, 1,1-dimethylbutyl, 3,3,3-trifluoropropyl, 4,4,4-trifluorobutyl or 1,1-dimethyl-3,3,3-trifluoropropyl group are new. Such compounds are useful in the synthesis of compounds of the general formula (I).

Compounds of the general formula (IV) wherein R and R₁ have the same meanings as given in claim 1 and R₂ represents an phenyl group which may be substituted with 1-5 substituents Y2 which can be the same or different, selected from the group C₁₋₃-alkoxy, hydroxy, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethyl-sulfonyl, cyano, carbamoyl, sulfamoyl, ortho-halogen, meta-halogen, ortho-C₁₋₃-alkyl, meta-C₁₋₃-alkyl and acetyl, or R₂ represents a thienyl or pyridyl group, which groups may be substituted with one or two substituents Y, which Y group has the meaning as in claim 1, are new. Such compounds are useful in the synthesis of compounds of formula (I).

Compounds of the general formula (VIII) wherein R and R₂ have the same meanings as given hereinabove and R₁ represents hydrogen are new. Such compounds are useful in the synthesis of compounds of the general formula (I) wherein n=1.

Pharmaceutically acceptable salts may be obtained using standard procedures well known in the art, for example by mixing a compound of the present invention with a suitable acid, for instance an inorganic acid such as hydrochloric acid, or with an organic acid such as fumaric acid.

According to these procedures the compounds described below have been prepared. They are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only.

### Example 3

### Synthesis and Spectral Data of Intermediates

Intermediate II-1

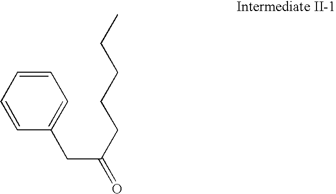

To a magnetically stirred solution of hexanoic acid methoxy-methyl-amide (12.2 g, 77 mmol) at 0° C. in tetrahydrofuran (THF) was slowly added benzylmagnesium chloride (20 weight percent solution in THF, 90 ml 116 mmol) and the resulting mixture was reacted for two hours. The reaction mixture was poured in excess aqueous hydrochloric acid (4N solution) and extracted with tert-butyl-methyl ether (MTBE). Concentration in vacuo, followed by flash chromatographic purification (heptane/ethylacetate=40/1 (v/v)) gave 1-phenylheptan-2-one (Intermediate II-1) (11.6 gram) as an oil; ¹H-NMR (300 MHz, CDCl₆) δ 0.86 (t, J=7, 3H), 1.20-1.27 (m, 4H), 1.52-1.60 (m, 2H), 2.40-2.46 (m, 2H), 3.68 (s, 2H), 7.18-7.33 (m, 5H).
Intermediate II-2

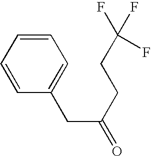

4,4,4-Trifluoro-N-methoxy-N-methylbutyramide (7.68 g) was obtained in 87% yield as an oil from the reaction of 4,4,4-trifluorobutyric acid (6.77 g, 0.0477 mol) with N-methyl-N-methoxy-amine.HCl in the presence of N-hydroxybenzotriazole (HOBt), 1-(3-dimethylaminopropyl)-3-ethylcarbodiimide.HCl (EDCl) and N-methylmorpholine (NMM) in dichloromethane as the solvent (room temperature, 16 hours). ¹H-NMR (400 MHz, CDCl₃) δ 2.40-2.54 (m, 2H), 2.67-2.73 (m, 2H), 3.20 (s, 3H), 3.71 (s, 3H). 4,4,4-Trifluoro-N-methoxy-N-methylbutyramide (7.68 g) was converted with benzylmagnesium chloride at 0° C. in tetrahydrofuran (THF) analogously to the procedure described for the synthesis of intermediate II-1 to give 6.37 gram (71%) 5,5,5-trifluoro-1-phenylpentan-2-one (Intermediate II-2). Chromatographic sepacore purification (petroleum ether/diethyl ether=47/1 (v/v)) was used to purify intermediate II-2. ¹H-NMR (400 MHz, CDCl₃) δ 2.31-2.44 (m, 2H), 2.68-2.75 (m, 2H), 3.73 (s, 2H), 7.18-7.38 (m, 5H).
Intermediate II-3

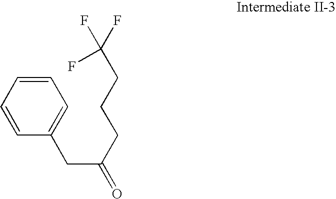

Intermediate II-3 (6,6,6-trifluoro-1-phenyl-hexan-2-one) was prepared analogously to intermediate II-1 from 5,5,5-trifluoropentanoic acid methoxymethyl-amide and benzylmagnesium chloride (20 weight percent solution in THF) at 0° C. in tetrahydrofuran as an oil; ¹H-NMR (400 MHz, CDCl₃) δ 1.75-1.85 (m, 2H), 1.98-2.11 (m, 2H), 2.55 (t, J=7, 2H), 3.69 (s, 2H), 7.18-7.22 (m, 2H), 7.26-7.37 (m, 3H).

5,5,5-Trifluoropentanoic acid methoxy-methyl-amide: ¹H-NMR (400 MHz, CDCl₃) δ 1.86-1.95 (m, 2H), 2.11-2.24 (m, 2H), 2.53 (br t, J=7, 2H), 3.19 (s, 3H), 3.69 (s, 3H). 5,5,5-Trifluoropentanoic acid methoxy-methyl-amide was obtained from the reaction of 5,5,5-trifluoropentanoic acid and N-methyl-N-methoxy-amine.HCl in the presence of N-hydroxybenzotriazole, 1-(3-dimethylaminopropyl)-3-ethylcarbodiimide.HCl and N-methylmorpholine in dichloromethane.
Intermediate II-4

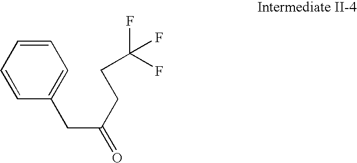

Intermediate II-4 (6,6,6-trifluoro-1-phenyl-pentan-2-one) was prepared analogously to intermediate II-1, from 4,4,4-trifluoro-N-methoxy-N-methyl-butyramide and benzylmagnesium chloride (20 weight percent solution in THF) at 0° C. in tetrahydrofuran as an oil; ¹H-NMR (400 MHz, CDCl₃) δ 2.31-2.44 (m, 2H), 2.71 (t, J=7, 2H), 3.73 (s, 2H), 7.18-7.22 (m, 2H), 7.26-7.38 (m, 5H).

4,4,4Trifluoro-N-methoxy-N-methyl-butyramide: ¹H-NMR (400 MHz, CDCl₃) δ 2.41-2.53 (m, 2H), 2.70 (br t, J=7, 2H), 3.20 (s, 3H), 3.71 (s, 3H). 4,4,4-Trifluoro-N-methoxy-N-methyl-butyramide was obtained from the reaction of 4,4,4-trifluorobutyric acid and N-methyl-N-methoxy-amine.HCl in the presence of N-hydroxybenzotriazole, 1-(3-dimethylaminopropyl)-3-ethylcarbodiimide. HCl and N-methylmorpholine in dichloromethane.
Intermediate II-5

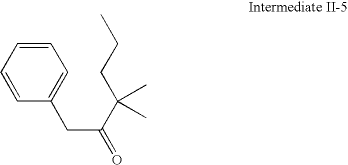

Intermediate II-5 (3,3-dimethyl-1-phenyl-hexan-2-one) was prepared analogously to intermediate II-1 from 2,2-dimethylpentanoic acid methoxy-methyl-amide and benzylmagnesium chloride (20 weight percent solution in THF) at 0° C. in tetrahydrofuran as an oil; ¹H-NMR (400 MHz, CDCl₃) δ 0.89 (t, J=7, 3H), 1.14-1.23 (m, 8H), 1.53-1.60 (m, 2H), 3.76 (s, 2H), 7.15-7.33 (m, 5H).

2,2-Dimethylpentanoic acid methoxy-methyl-amide: ¹H-NMR (400 MHz, CDCl₃) δ 0.90 (t, J=7, 3H), 1.20-1.29 (m, 8H), 1.55-1.60 (m, 2H), 3.17 (s, 3H), 3.67 (s, 3H). 2,2-Dimethylpentanoic acid methoxy-methyl-amide was obtained from the reaction of 2,2-dimethylpentanoic acid and N-methyl-N-methoxy-amine.HCl in the presence of N-hydroxybenzotriazole, 1-(3-dimethylaminopropyl)-3-ethylcarbodiimide.HCl and N-methylmorpholine in dichloromethane.
Intermediate II-6

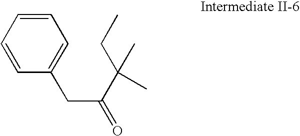

Intermediate II-6 (3,3-dimethyl-1-phenyl-pentan-2-one) was prepared analogously to intermediate II-1 from 2,2,N-trimethyl-N-methoxy-butyramide and benzylmagnesium chloride (20 weight percent solution in THF) at 0° C. in tetrahydrofuran as an oil; ¹H-NMR (400 MHz, CDCl₃) δ 0.81 (t, J=7, 3H), 1.15 (s, 6H), 1.64 (q, J=7.5, 2H), 3.76 (s, 2H), 7.15-7.33 (m, 5H).

2,2,N-Trimethyl-N-methoxy-butyramide: ¹H-NMR (400 MHz, CDCl₃) δ 0.85 (t, J=7, 3H), 1.21 (s, 6H), 1.61-1.69 (m, 2H), 3.18 (s, 3H), 3.67 (s, 3H). 2,2,-Trimethyl-N-methoxy-butyramide was obtained from the reaction of 2,2-dimethylbutyric acid and N-methyl-N-methoxy-amine.HCl in the presence of N-hydroxybenzotriazole, 1-(3-dimethylaminopropyl)-3-ethylcarbodiimide.HCl and N-methylmorpholine in dichloromethane.
Intermediate II-7

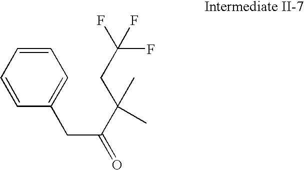

Intermediate II-7 (3,3-dimethyl-5,5,5-trifluoro-1-phenyl-pentan-2-one) was prepared analogously to intermediate II-1 from 4,4,4-trifluoro-2,2,N-trimethyl-N-methoxy-butyramide and benzylmagnesium chloride (20 weight percent solution in THF) at 0° C. in tetrahydrofuran as an oil; ¹H-NMR (400 MHz, CDCl₃) δ 1.34 (s, 6H), 2.47 (d, J˜12, 1H), 2.52 (d, J˜12, 1H), 3.84 (s, 2H), 7.15 (br d, J˜8, 2H), 7.23-7.36 (m, 3H).

4,4,4-Trifluoro-2,2,N-trimethyl-N-methoxy-butyramide: ¹H-NMR (400 MHz, CDCl₃) δ 1.35 (s, 6H), 2.55 (d, J˜12, 1H), 2.60 (d, J˜12, 1H), 3.19 (s, 3H), 3.70 (s, 3H). 4,4,4-Trifluoro-2,2,N-trimethyl-N-methoxy-butyramide was obtained from the reaction of 4,4,4-trifluoro-2,2-dimethylbutyric acid and N-methyl-N-methoxy-amine.HCl in the presence of N-hydroxybenzotriazole, 1-(3-dimethylaminopropyl)-3-ethylcarbodiimide.HCl and N-methylmorpholine in dichloromethane.
Intermediate II-8

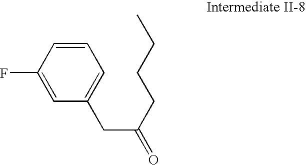

3-Fluorobenzyl bromide (25 g, 0.132 mol) was converted to 3-fluorobenzyl magnesiumbromide in anhydrous diethyl ether (85 ml) using magnesium (3.17 g) in the presence of catalytic amounts of iodine and 1,2-dibromoethane. The in situ formed 3-fluorobenzyl magnesium bromide was reacted with pentanenitrile (11 ml) in toluene (100 ml) at 110° C. for 2 hours. After hydrolysis of the formed mixture with concentrated hydrochloric acid (12 N) at 80° C. for 4 hours and subsequent extraction with toluene 1-(3-fluorophenyl)-hexan-2-one was obtained in 86% yield as an oil. ¹H-NMR (400 MHz, CDCl₃) δ 0.87 (t, J=7, 3H), 1.22-1.33 (m, 2H), 1.50-1.59 (m, 2H), 2.46 (t, J=7, 2H), 3.68 (s, 2H), 6.90-7.00 (m, 3H), 7.26-7.32 (m, 2H).
Intermediate II-9

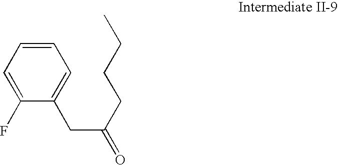

2-Fluorobenzyl bromide was converted to 2-fluorobenzyl magnesiumbromide in anhydrous diethyl ether using magnesium in the presence of catalytic amounts of iodine and 1,2-dibromoethane analogously to the procedure described for the synthesis of Intermediate II-8. The in situ formed 2-fluorobenzyl magnesium bromide was reacted with pentanenitrile in toluene at 110° C. for 2 hours. After hydrolysis of the formed mixture with concentrated hydrochloric acid (12 N) at 80° C. for 20 hours 1-(2-fluorophenyl)-hexan-2-one was obtained in 70% yield as an oil. ¹H-NMR (400 MHz, CDCl₃) δ 0.88 (t, J=7, 3H), 1.24-1.35 (m, 2H), 1.53-1.62 (m, 2H), 2.48 (t, J=7, 2H), 3.72 (br s, 2H), 6.98-7.28 (m, 4H).
Intermediate II-10

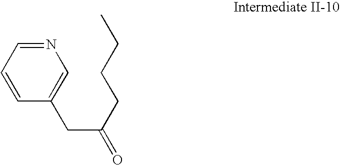

To a magnetically stirred solution of N-methoxy-N-methyl-2-(pyridin-3-yl)acetamide (12 g, 67 mmol) at −15° C. in tetrahydrofuran (THF) was slowly added n-butylmagnesium chloride (2 M solution in THF, 75 ml, 150 mmol) and the resulting mixture was reacted for 1 hour at −15° C. and successively stirred at room temperature overnight. The reaction mixture was poured in excess aqueous NH₄Cl and extracted twice with ethylacetate. Concentration in vacuo, followed by sepacore chromatographic purification (ethylacetate) gave 1-(pyridin-3-yl)hexan-2-one (Intermediate II-10) (5.95 gram, 50% yield) as an oil; ¹H-NMR (400 MHz, CDCl₆) δ 0.89 (t, J=7, 3H), 1.24-1.35 (m, 2H), 1.52-1.62 (m, 2H), 2.50 (t, J=7, 2H), 3.70 (s, 2H), 7.25-7.29 (m, 1H), 7.52-7.57 (m, 1H), 8.45 (br d, J=2, 1H), 8.52 (dd, J˜6 and 2, 1H).
Intermediate III-1

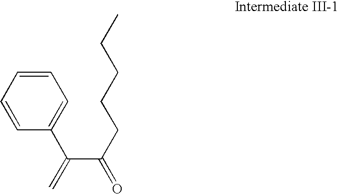

To a magnetically stirred solution of 1-phenylheptan-2-one (Intermediate II-1) (11.6 gram, 61 mmol) in methanol (100 ml) was added piperidine (1 ml) and acetic acid (1 ml), followed by a formaldehyde solution (20 ml of a 35% solution in water, 226 mmol) and the resulting mixture was stirred at 55° C. for 60 hours. The reaction mixture was cooled to room temperature, concentrated and taken up in a mixture of MTBE and water. The organic layer was collected, dried over Na₂SO₄, filtered and concentrated to give 2-phenyl-oct-1-en-3-one (Intermediate III-1) (11.4 gram) as an oil. Intermediate III-1: ¹H-NMR (400 MHz, CDCl₃) δ 0.80 (t, J=7, 3H), 1.18-1.30 (m, 4H), 1.54-1.63 (m, 2H), 2.65 (t, J=7, 2H), 5.80 (s, 1H), 6.02 (s, 1H), 7.20-7.32 (m, 5H).
Intermediate III-2

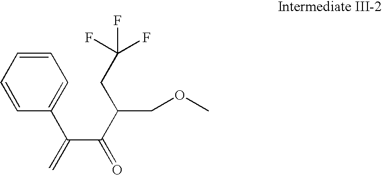

5,5,5-Trifluoro-1-phenylpentan-2-one (Intermediate II-2) was reacted in methanol with piperidine and acetic acid, followed by a formaldehyde solution (35% solution in water) and the resulting mixture was stirred at 55° C. for 60 hours analogously to the procedure described for the synthesis of intermediate III-1 to give 6,6,6-trifluoro-4-methoxymethyl-2-phenyl-hex-1-en-3-one (intermediate III-2) in 16% yield. Chromatographic sepacore purification (petroleum ether/diethyl ether=19/1 (v/v)) was used to purify intermediate III-2 ¹H-NMR (400 MHz, CDCl₃) □ 2.28-2.42 (m, 1H), 2.70-2.85 (m, 1H), 3.29 (s, 3H), 3.47-3.60 (m, 2H), 3.68-3.76 (m, 1H), 6.01 (s, 1H), 6.13 (s, 1H), 7.28-7.40 (m, 5H).
Intermediate III-3

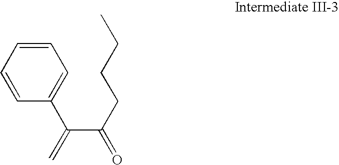

Intermediate III-3 (2-phenyl-hept-1-en-3-one) was prepared analogously to intermediate III-1, from 1-phenylhexan-2-one, piperidine, acetic acid and formaldehyde solution (35% solution in water) at 55° C. for 60 hours. Intermediate III-3: ¹H-NMR (400 MHz, CDCl₃) δ 0.91 (t, J=7, 3H), 1.30-1.40 (m, 2H), 1.59-1.69 (m, 2H), 2.73 (t, J=7, 2H), 5.87 (s, 1H), 6.09 (s, 1H), 7.28-7.40 (m, 5H).
Intermediate III-4

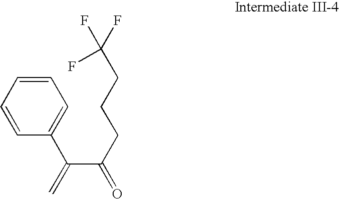

Intermediate III-4 (7,7,7-trifluoro-2-phenyl-hept-1-en-3-one) was prepared analogously to intermediate III-1, from 6,6,6-trifluoro-1-phenylhexan-2-one, piperidine, acetic acid and formaldehyde solution (35% solution in water) at 55° C. for 60 hours. Intermediate III-4: ¹H-NMR (400 MHz, CDCl₃) δ 1.89-1.98 (m, 2H), 2.09-2.22 (m, 2H), 2.84 (t, J=7, 2H), 5.91 (s,.1H), 6.13 (s, 1H), 7.26-7.40 (m, 5H).
Intermediate III-5

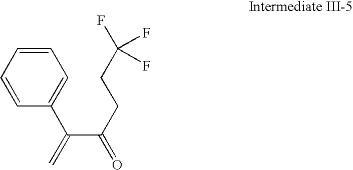

Intermediate III-5 (6,6,6-trifluoro-2-phenyl-hex-1-en-3-one) was prepared analogously to intermediate III-1 with some modifications (temperature and amount of formaldehyde used), from 5,5,5-trifluoro-1-phenylpentan-2-one, piperidine, acetic acid and formaldehyde solution (1.1 molar equivalent CH₂O, 35% solution in water) at 40° C. for 40 hours in 57% yield. Purification was performed by sepacore chromatographic purification (petroleum ether/diethyl ether=39/1 (v/v)). R_{f}=0.4 (petroleum ether/diethyl ether=9/1 (v/v)). Intermediate III-5: ¹H-NMR (400 MHz, CDCl₃) δ 2.43-2.56 (m, 2H), 3.03 (t, J=7, 2H), 5.97 (s, 1H), 6.19 (s, 1H), 7.26-7.40 (m, 5H).
Intermediate III-6

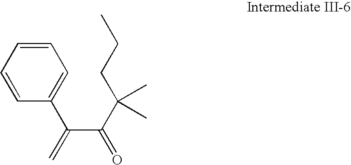

Intermediate III-6 (4,4-dimethyl-2-phenyl-hept-1-en-3-one) was prepared analogously to intermediate III-1, from 3,3-dimethyl-1-phenylhexan-2-one, piperidine, acetic acid and formaldehyde solution (35% solution in water) at 55° C. for 60 hours. Intermediate III-6: ¹H-NMR (400 MHz, CDCl₃) δ 0.83 (t, J=7, 3H), 1.02 (s, 6H), 1.10-1.19 (m, 2H), 1.40-1.50 (m, 2H), 5.13 (s, 1H), 5.45 (s, 1H), 7.09-7.38 (m, 5H).
Intermediate III-7

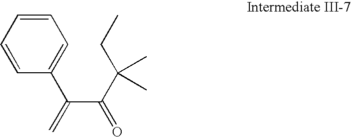

Intermediate III-7 (4,4-dimethyl-2-phenyl-hex-1-en-3-one) was prepared analogously to intermediate III-1, from 3,3-dimethyl-1-phenylpentan-2-one, piperidine, acetic acid and formaldehyde solution (35% solution in water) at 55° C. for 60 hours. Intermediate III-7: ¹H-NMR (400 MHz, CDCl₃) δ 0.81 (t, J=7, 3H), 1.09 (s, 6H), 1.59 (q, J=7, 2H), 5.20 (s, 1H), 5.52 (s, 1H), 7.29-7.37 (m, 5H).
Intermediate III-8

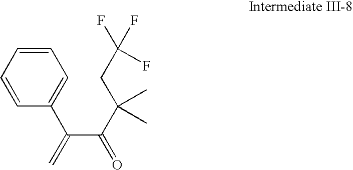

Intermediate III-8 (4,4-dimethyl-6,6,6-trifluoro-2-phenyl-hex-1-en-3-one) was prepared analogously to intermediate III-1, from 3,3-dimethyl-5,5,5-trifluoro-1-phenylpentan-2-one, piperidine, acetic acid and formaldehyde solution (35% solution in water) at 55° C. for 60 hours. Intermediate III-8: ¹H-NMR (400 MHz, CDCl₃) δ 1.22 (s, 6H), 2.49 (d, J˜12, 1H), 2.56 (d, J-12, 1H), 5.29 (s, 1H), 5.57 (s, 1 H), 7.29-7.39 (m, 5H).
Intermediate III-9

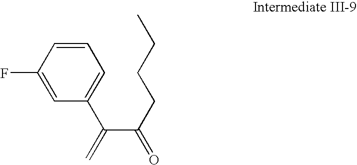

Intermediate III-9 (2-(3-fluorophenyl)-hept-1-en-3-one) was prepared analogously to intermediate III-1, from 1-(3-fluorophenyl)-hexan-2-one, piperidine, acetic acid and formaldehyde solution (35% solution in water) at 55° C. for 60 hours. Intermediate III-9: ¹H-NMR (400 MHz, CDCl₃) δ 0.93 (t, J=7, 3H), 1.30-1.41 (m, 2H), 1.60-1.69 (m, 2H), 2.75 (t, J =7, 2H), 5.93 (s, 1H), 6.15 (s, 1H), 7.00-7.09 (m, 3H) 7.28-7.35 (m, 1H).
Intermediate III-10

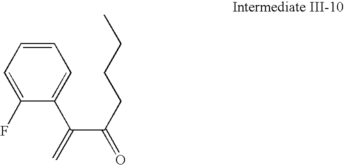

Intermediate III-10 (2-(2-fluorophenyl)-hept-1-en-3-one) was prepared analogously to intermediate III-1, from 1-(2-fluorophenyl)-hexan-2-one, piperidine, acetic acid and formaldehyde solution (35% solution in water) at 55° C. for 60 hours. Intermediate III-10: ¹H-NMR (400 MHz, CDCl₃) δ 0.91 (t, J=7, 3H), 1.30-1.40 (m, 2H), 1.59-1.69 (m, 2H), 2.70 (t, J=7, 2H), 5.88 (s, 1H), 6.26 (s, 1H), 6.98-7.37 (m, 4H).
Intermediate III-11

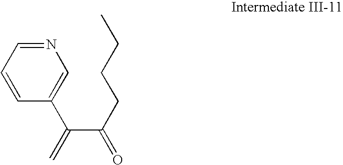

To a magnetically stirred, ice-cooled solution of 1-(pyridin-3-yl)hexan-2-one (6 g, 34 mmol) and N,N,N′,N′-tetramethyidiaminomethane (7 ml, 51 mmol) at 0° C. was slowly added acetic anhydride (Ac₂O) (4.8 ml, 51 mmol). The resulting mixture was reacted for 30 minutes at 45° C. and successively cooled to room temperature. The reaction mixture was poured in excess ice and brine was added. Extraction with ethylacetate (2×) and dichloromethane followed by drying (Na₂SO₄) of the combined organic layers, filtering and concentration in vacuo gave crude product. Subseqent sepacore chromatographic purification (ethylacetate) gave 2-(pyridin-3-yl)hept-1-en-3-one (Intermediate III-11) (3.86 gram, 60% yield); ¹H-NMR (400 MHz, CDCl₆) δ 0.93 (t, J =7, 3H), 1.32-1.43 (m, 2H), 1.62-1.71 (m, 2H), 2.81 (t, J=7, 2H), 6.06 (s, 1H), 6.28 (s, 1H), 7.25-7.31 (m, 1H), 7.63-7.68 (m, 1H), 8.53-8.58 (m, 2H).
Intermediate III-12

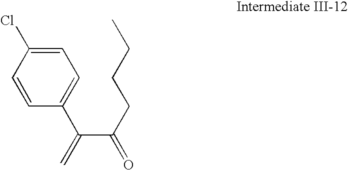

Intermediate III-12 (2-(4-chlororophenyl)-hept-1-en-3-one) was prepared analogously to intermediate III-1, from 1-(4-chlorophenyl)-hexan-2-one, piperidine, acetic acid and formaldehyde solution (35% solution in water) at 55° C. for 60 hours. Intermediate III-12: ¹H-NMR (400 MHz, CDCl₃) δ 0.92 (t, J=7, 3H), 1.30-1.41 (m, 2H), 1.59-1.68 (m, 2H), 2.74 (t, J=7, 2H), 5.92 (s, 1H), 6.13 (s, 1H), 7.24 (br d, J=8, 2H), 7.32 (br d, J=8, 2H).
Intermediate III-13

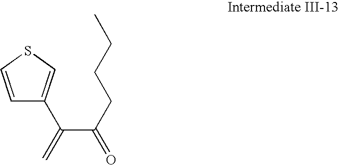

Intermediate III-13 (2-(thien-3-yl)-hept-1-en-3-one) was prepared analogously to intermediate III-1, from 1-(thien-3-yl)hexan-2-one, piperidine, acetic acid and formaldehyde solution (35% solution in water) at 55° C. for 60 hours. Intermediate III-13: ¹H-NMR (400 MHz, CDCl₃) δ 0.93 (t, J=7, 3H), 1.31-1.42 (m, 2H), 1.61-1.69 (m, 2H), 2.77 (t, J˜8, 2H), 6.03 (s, 1H), 6.04 (s, 1H), 7.18 (dd, J=6 and 2, 1H), 7.28 (dd, J˜6 and 3, 1H), 7.51-7.53 (m, 1H).
Intermediate IV-2

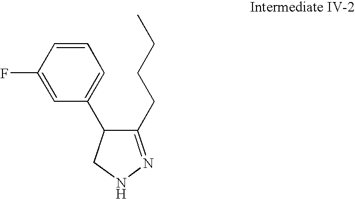

Intermediate IV-2 (3-(n-butyl)-4-(3-fluorophenyl-4,5-dihydropyrazole) was prepared analogously to 3-(n-pentyl)-4-phenyl-4,5-dihydropyrazole (Intermediate IV-1, see preparation of compound 1), from 2-(3-fluorophenyl)-hept-1-en-3-one and hydrazine hydrate. Some characteristic pyrazoline ring proton NMR signals: (400 MHz, CDCl₃) δ 3.37 (t, J˜10, 1H, H₅), 3.81 (t, J˜10, 1H, H₅), 3.99 (t, J˜9, 1H, H₄).
Intermediate IV-3

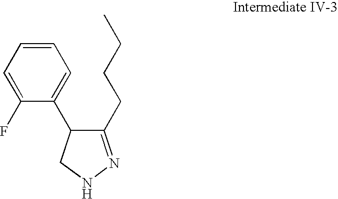

Intermediate IV-3 (3-(n-butyl)-4-(2-fluorophenyl-4,5-dihydropyrazole) was prepared analogously to 3-(n-pentyl)-4-phenyl-4,5-dihydropyrazole (Intermediate IV-1), from 2-(2-fluorophenyl)-hept-1-en-3-one and hydrazine hydrate. Some characteristic pyrazoline ring proton NMR signals: (400 MHz, CDCl₃) δ 3.37 (t, J˜9, 1H, H₅), 3.78 (t, J˜10, 1H, H₅), 4.35 (t, J˜10, 1H, H₄).
Intermediate VII-1

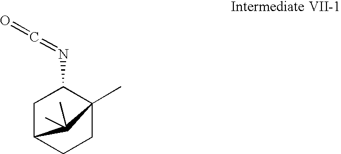

To a magnetically stirred solution of diphosgene (4.26 ml, 0.0353 mol) in dichloromethane (90 ml) was slowly added a solution of endo-1R, 2S, 4R-)-1,7,7-trimethylbicyclo[2.2.1]hept-2-ylamine (CAS 32511-34-5) and N,N-dimethylaniline (15.2 ml, 0.12 mol)) in dichloromethane (90 ml) at 0° C. The resulting mixture was allowed to attain room temperature and stirred for 30 minutes. The mixture was concentrated and the residue taken up in dichloromethane, washed (3× with 1N HCl and 1× brine), dried (MgSO₄), filtered and concentrated in vacuo to give endo-2-isocyanato-[(1R,2S,4R)-1,7,7-trimethylbicyclo[2.2.1]heptane (10.43 g, 97% yield. ¹H-NMR (400 MHz, CDCl₃) δ 0.85 (s, 3H), 0.86 (s, 3H), 0.89 (s, 3H), 1.11 (dd, J=13.2 and 4.2, 1H), 1.21-1.28 (m, 1H), 1.30-1.38 (m, 1H), 1.67 (t, J=4, 1H), 1.71-1.83 (m, 2H), 2.26-2.34 (m, 1H), 3.75 (ddd, J=10.5, 4.1 and 2.3, 1H). Optical rotation ([α]_{D})=+40.2 (c=1.07, dichloromethane).
Intermediate VII-2

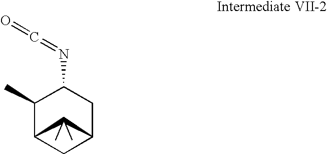

3-lsocyanato-[(1R,2R,3R,5S)-2,7,7-trimethylbicyclo[3.1.1]heptane (intermediate VII-2) was prepared from the reaction of (−)-3-amino-[(1R,2R,3R,5S)-2,7,7-trimethylbicyclo[3.1.1]heptane (CAS 69460-11-3) and triphosgene in the presence of DIPEA in dichloromethane at 0° C. ¹H-NMR (400 MHz, CDCl₃) δ 0.95 (s, 3H), 1.00 (d, J=9, 1H), 1.13 (d, J=7, 3H), 1.23 (s, 3H), 1.80-1.90 (m, 2H), 1.93-2.00 (m, 1H), 2.04-2.13 (m, 1H), 2.38-2.44 (m, 1H), 2.49-2.58 (m, 1H), 3.80-3.88 (m, 1H).
Intermediate VII-3

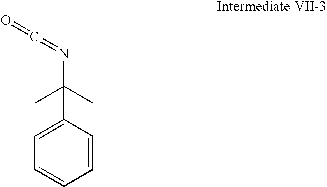

Intermediate VII-3 was prepared from diphosgene, cumylamine and N,N-dimethylaniline in dichloromethane analogously to the procedure described for intermediate VII-1. ¹H-NMR (400 MHz, CDCl₃) δ 1.71 (s, 6H), 7.22-7.29 (m, 1H), 7.32-7.38 (m, 2H), 7.42-7.46 (m, 2H).
Intermediate VII-4

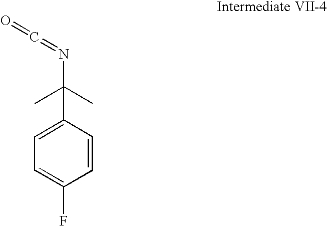

Intermediate VII-4 was prepared from diphosgene, 1-(4-fluorophenyl)-1-(methyl)ethylamine and N,N-dimethylaniline in dichloromethane analogously to the procedure described for intermediate VII-1. ¹H-NMR (400 MHz, CDCl₃) δ 1.70 (s, 6H), 6.99-7.05 (m, 2H), 7.37-7.43 (m, 2H).

### 3-(n-Butyl)-4-(2-fluorophenyl)-4,5-dihydro-(1H)-pyrazole-1-carbonyl chloride

Intermediate VIII-1

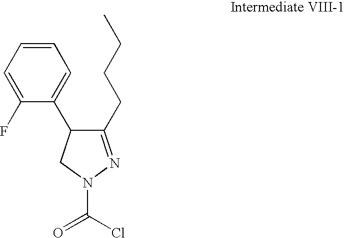

To a magnetically stirred solution of crude 3-(n-butyl)-4-(2-fluorophenyl)-4,5-dihydro-(1H)-pyrazole (Intermediate (IV-3) (2.0 gram, 8.93 mmol maximally) in dichloromethane (25 ml) was successively added DIPEA (1.50 g, 2.0 ml, 11.61 mmol) and triphosgene (0.79 g, 2.68 mmol, dissolved in 10 ml dichloromethane) at 0° C. and the resulting solution was allowed to attain room temperature and subsequently reacted at room temperature for 1 hour. Column chromatographic purification (eluant: dichloromethane) gave pure 3-(n-butyl)-4-(2-fluorophenyl )-4,5-dihydro-(1H pyrazole-1-carbonyl chloride (Intermediate VIII-1) (1.26 g, ˜50% yield). ¹H-NMR (400 MHz, CDCl₃) δ 0.86 (t, J=7, 3H), 1.22-1.36 (m, 2H), 1.42-1.60 (m, 2H), 2.08-2.18 (m, 1H), 2.27-2.40 (m, 1H), 3.96 (dd, J=12 and 7, 1H), 4.34 (t, J=12, 1H), 4.54-4.64 (m, 1H), 7.08-7.22 (m, 3H), 7.30-7.38 (m, 1H).

### 3-(n-pentyl)-4-phenyl-4,5-dihydro-(1H)-pyrazole-1-carbonyl chloride

Intermediate VIII-2

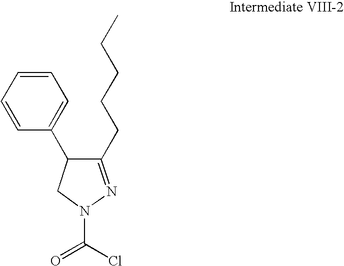

To a magnetically stirred solution of 3-(n-pentyl)-4-phenyl-4,5-dihydro-(1H)-pyrazole (116 ml of a 0.25 M solution in dichloromethane) was added DIPEA (116 ml of a 0.30 M solution in dichloromethane) and triphosgene (0.3 mol equivalent as a solution in dichloromethane) at 0 ° C and the resulting solution was allowed to attain room temperature and subsequently reacted at room temperature for 1 hour to give a stock solution of crude 3-(n-pentyl)-4-phenyl-4,5-dihydro-(1H)-pyrazole-1-carbonyl chloride (Intermediate VIII-2). This stock solution was used in parallel reactions with various amines, to prepare compounds 103-123.

### Example 4

### Synthesis of Specific Compounds

### Compound 1

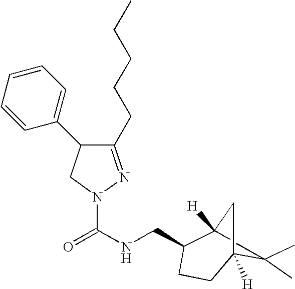

### N-[(1R,2S,5R)-rel-6,6-dimethylbicyclo[3.1.1]heptan-2-methyl]-3-(n-pentyl)-4-phenyl-4,5-dihydro-(1H)-pyrazole-1-carboxamide

Part A: To a magnetically stirred solution of 2-phenyl-oct-1-en-3-one (Intermediate III-1) (5 gram, 24.7 mmol) in ethanol (30 ml) was added hydrazine hydrate (2.46 ml, 50.7 mmol) and the resulting solution was heated at reflux temperature for 4 hours. The resulting solution was allowed to attain room temperature, concentrated and taken up in a mixture of MTBE and water. The organic layer was collected, dried over Na₂SO₄, filtered and concentrated to give crude 3-(n-pentyl)-4-phenyl-4,5-dihydropyrazole (Intermediate IV-1) (4.8 gram) as an impure oil which was used immediately in the subsequent step. (Intermediate IV-1) some characteristic pyrazoline ring proton NMR signals: (400 MHz, CDCl₃) δ 3.36 (t, J˜10, 1H), 3.81 (t, J˜10, 1H), 4.00 (t, J˜10, 1H).

Part B: To a magnetically stirred solution of (−)-cis-myrtanylamine (2.4 ml, 14.2 mmol) (CAS 38235-68-6)) in dichloromethane (40 ml) was added triethylamine (2 ml, 14.2 mmol). The resulting solution was slowly added to a solution of triphosgene (1.4 gram, 4.7 mmol) in dichloromethane (60 ml) and the resulting mixture was stirred at room temperature for 16 hours. The mixture was then poured in water and extracted with dichloromethane, dried over Na₂SO₄, filtered and concentrated to give cis-myrtanylisocyanate (2.12 gram) as an oil.

Part C. 3-(n-Pentyl-4-phenyl-4,5-dihydropyrazole (2.2 gram, 10.3 mmol) was dissolved in benzene (25 ml) and treated with cis-myrtanylisocyanate (2.12 g, 11.8 mmol) and 5 drops of triethylamine and the resulting solution was stirred at room temperature for 16 hours. The solution was concentrated, followed by flash chromatographic purification (heptane/ethylacetate=6:1 (v/v)) to give N-[(1R,2S,5R)-rel-6,6-dimethylbicyclo[3.1.1]heptan-2-methyl]-3-(n-pentyl)-4-phenyl-4,5-dihydro-(1H)-pyrazole-1-carboxamide] as an oil. ¹H-NMR (400 MHz, CDCl₃) δ 0.85-0.95 (m, 4H), 1.06 (s, 3H), 1.19-1.31 (m, 7H), 1.38-1.60 (m, 3H), 1.82-2.41 (m, 9H), 3.22-3.40 (m, 2H), 3.83-3.90 (m, 1H), 4.12 (dd, J=12 and 7, 1H), 4.18-4.26 (m, 1H), 5.92-5.96 (m, 1H), 7.15 (br d, J˜8, 2H), 7.25-7.37 (m, 3H). LC/MS (Method A). Retention time: 7.07 minutes: Found molecular mass (API-ES; positive scan)=396.

Analogously the compounds 2-84 were prepared:

### Compound 2

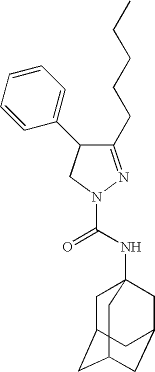

### N-(1-Adamantyl)-3-(n-pentyl)-4-phenyl-4,5-dihydro-(1H)-pyrazole-1-carboxamide

LC/MS (Method A). Retention time: 8.04 minutes: Found molecular mass (API-ES; positive scan)=394. R_{f} (dichloromethane/methanol=99/1 (v/v))=0.3.

### Compound 3

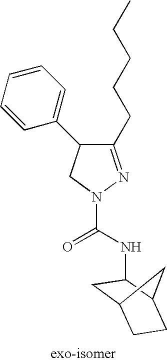

### N-(Exo-bicyclo[2.2.1]hept-2-yl)-3-(n-pentyl)-4-phenyl-4,5-dihydro-(1H)-pyrazole-1-carboxamide

LC/MS (Method A). Retention time: 9.26 minutes: Found molecular mass (API-ES; positive scan)=354. R_{f} (dichloromethane/methanol=99/1 (v/v))=0.2.

### Compound 4

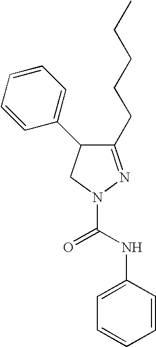

### N-Phenyl-3-(n-pentyl)-4phenyl-4,5-dihydro-(1H)-pyrazole-1-carboxamide

LC/MS (Method A). Retention time: 4.35 minutes: Found molecular mass (API-ES; positive scan)=336. R_{f} (dichloromethane/methanol=99/1 (v/v))=0.4.

### Compound 5

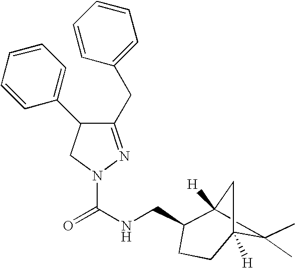

### N-[(1R,2S,5R)-rel-6,6-dimethylbicyclo[3.1.1]heptan-2-methyl]-3-(benzyl)-4-phenyl-4,5-dihydro-(1H)-pyrazole-1-carboxamide (from the isocyanate derived from (−)-cis-myrtanylamine (CAS 38235-68-6))

LC/MS (Method A). Retention time: 4.96 minutes: Found molecular mass (API-ES; positive scan)=416. R_{f} (dichloromethane/methanol=99/1 (v/v))=0.25.

### Compound 6

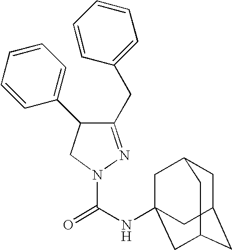

### N-(1-Adamantyl)-3-(benzyl)-4-phenyl-4,5-dihydro-(1H)-pyrazole-1-carboxamide

¹H-NMR (400 MHz, CDCl₃) δ 1.65-1.75 (m, 6H), 2.06-2.13 (m, 9H), 3.20 (d, J˜14, 1H), 3.65 (d, J˜14, 1H), 3.84 (dd, J˜11 and 6, 1H), 3.95-4.00 (m, 1H), 4.14 (t, J˜11, 1H), 5.85 (br s, 1H), 7.05-7.11 (m, 4H), 7.22-7.36 (m, 6H).

LC/MS (Method A). Retention time: 5.34 minutes: Found molecular mass (API-ES; positive scan)=414. Melting point: 61° C.

### Compound 7

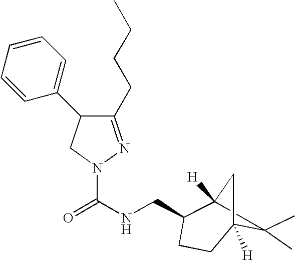

### N-[(1R,2S,5R)-rel-6,6-dimethylbicyclo[3.1.1]heptan-2-methyl]-3-(n-butyl)-4-phenyl-4,5-dihydro-(1H)-pyrazole-1-carboxamide (from the isocyanate derived from (−)-cis-myrtanylamine (CAS 38235-68-6))

LC/MS (Method B). Retention time: 5.03 minutes: Found molecular mass (API-ES; positive scan)=382. R_{f} (dichloromethane/methanol=99/1 (v/v))=0.2.

¹H-NMR (400 MHz, CDCl₃) δ 0.80-0.90 (m, 3H), 0.92 (d, J˜10, 1H), 1.06 (s, 3H), 1.21 (s, 3H), 1.22-1.60 (m, 5H), 1.82-2.41 (m, 8H), 3.23-3.40 (m, 2H), 3.87 (ddd, J˜11, 7 and 2, 1H), 4.12 (br dd, J˜11 and 7, 1H), 4.18-4.26 (m, 1H), 5.95 (br t, J˜7, 1H), 7.15 (br d, J˜8, 2H), 7.25-7.37 (m, 3H).

### Compound 8

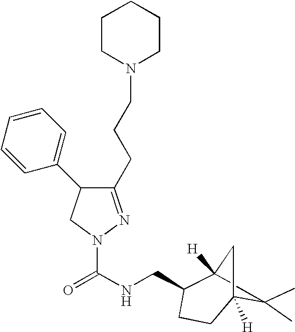

### N-[(1R,2S,5R)-rel-6,6-dimethylbicyclo[3.1.1]heptan-2-methyl]-3-[3-(1-piperidinyl)propyl]-4-phenyl-4,5-dihydro-(1H)-pyrazole-1-carboxamide (from the isocyanate derived from (−)cis-myrtanylamine (CAS 38235-68-6))

¹H-NMR (400 MHz, CDCl₃) δ 0.92 (d, J =10, 1H), 1.07 (s, 3H), 1.21 (s, 3H), 1.38-2.43 (m, 24H), 3.21-3.38 (m, 2H), 3.84-3.90 (m, 1H), 4.13 (dd, J=11 and 6, 1H), 4.19-4.26 (m, 1H), 5.97 (br t, J˜7, 1H), 7.15 (br d, J˜8, 2H), 7.25-7.40 (m, 3H).

### Compound 9

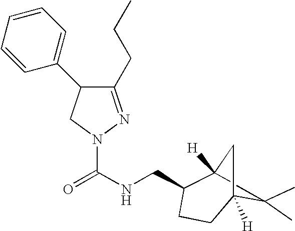

### N-[(1R,2S,5R)-rel-6,6-dimethylbicyclo[3.1.1]heptan-2-methyl]-3-(n-propyl)-4-phenyl-4,5-dihydro-(1H)-pyrazole-1-carboxamide (from the isocyanate derived from (−)-cis-myrtanylamine (CAS 38235-68-6))

¹H-NMR (400 MHz, CDCl₃) δ 0.85-0.95 (m, 4H), 1.16 (s, 3H), 1.21 (s, 3H), 1.41-1.61 (m, 2H), 1.83-2.17 (m, 8H), 2.25-2.41 (m, 2H), 3.22-3.39 (m, 2H), 3.83-3.90 (m, 1H), 4.12 (dd, J=12 and 7, 1H), 4.18-4.26 (m, 1H), 5.93-5.99 (m, 1H), 7.15 (br d, J˜8, 2H), 7.26-7.36 (m, 3H).

### Compound 10

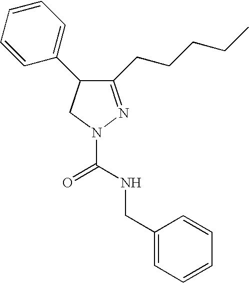

### N-(Benzyl)-3-(n-pentyl)-4-phenyl-4,5-dihydro-(1H)-pyrazole-1-carboxamide

LC/MS (Method B). Retention time: 5.76 minutes: Found molecular mass (API-ES; positive scan)=350. Mobile phase gradient: 0-5 minutes: Solution A/Solution B=30/70 (v/v)).>5 minutes: Solution B. R_{f} (dichloromethane/methanol=99/1 (v/v))=0.2.

### Compound 11

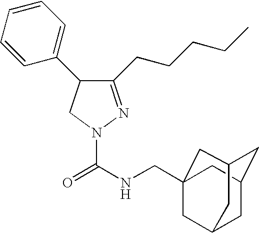

### N-(1-Adamantyl)methyl-3-(n-pentyl)-4-phenyl-4,5-dihydro-(1H)-pyrazole-1-carboxamide

LC/MS (Method B). Retention time: 6.28 minutes: Found molecular mass (API-ES; positive scan)=408. Mobile phase gradient: 0-3 minutes: Solution A/Solution B=20/80 (v/v)).>3 minutes: Solution B. R_{f}(dichloromethane/methanol=99/1 (v/v))=0.2.

### Compound 12

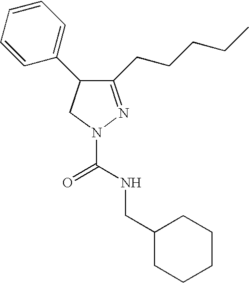

### N-(Cyclohexylmethyl)-3-(n-pentyl)-4-phenyl-4,5-dihydro-(1H)-pyrazole-1-carboxamide

LC/MS (Method B). Retention time: 7.01 minutes: Found molecular mass (API-ES; positive scan)=356. Mobile phase gradient: 0-5 minutes: Solution A/Solution B=30/70 (v/v)).>5 minutes: Solution B. R_{f}(dichloromethane/methanol=99/1 (v/v))=0.2.

### Compound 13

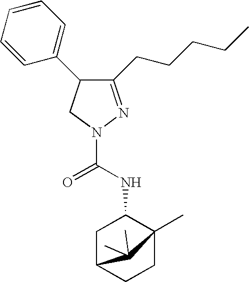

### N-[Endo-(1R,2S,4R)-1,7,7-trimethylbicyclo[2.2.1]hept-2-yl]-3-(n-pentyl)-4-phenyl-4,5-dihydro-(1H)-pyrazole-1-carboxamide (from the isocyanate (intermediate VII-1) derived from 1R-(+)-bornylamine (CAS 32511-34-5))

¹H-NMR (400 MHz, CDCl₃) δ 0.80-0.94 (m, 10H), 0.97 (s, 3H), 1.20-1.69 (m, 10H), 1.74-1.83 (m, 1H), 2.00-2.22 (m, 2H), 2.33-2.45 (m, 1H), 3.83-3.89 (m, 1H), 4.09-4.27 (m, 3H), 6.02 (br d, J˜10, 1H), 7.16 (br d, J˜8, 2H), 7.27-7.37 (m, 3H).

LC/MS (Method B). Retention time: 7.43 minutes: Found molecular mass (API-ES; positive scan)=396. Mobile phase gradient: 0-3 minutes: Solution A/Solution B=20/80 (v/v)).>3 minutes: Solution B. R_{f}(dichloromethane/methanol=99/1 (v/v))=0.3.

### Compound 14

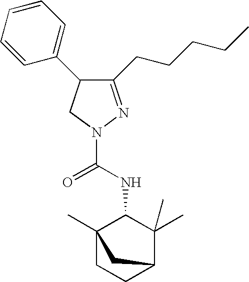

### N-[endo-(1S)-1,3,3-trimethylbicyclo[2.2.1]hept-2-yl]-3-(n-pentyl)-4phenyl-4,5-dihydro-(1H)-pyrazole-1-carboxamide (from the isocyanate derived from endo-(1S)-1,3,3-trimethylbicyclo[2.2.1]heptan-2-amine (CAS 301822-76-4)

LC/MS (Method B). Retention time: 5.83 minutes: Found molecular mass (API-ES; positive scan)=396. Mobile phase gradient: 0-5 minutes: Solution A/Solution B=15/85 (v/v)).>5 minutes: Solution B. R_{f}(dichloromethane/methanol=99/1 (v/v))=0.3.

### Compound 15

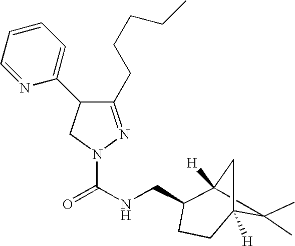

### N-[(1R,2S,5R)-rel-6,6-dimethylbicyclo[3.1.1]heptan-2-methyl]-3-(n-propyl)-4-(2-pyridyl)-4,5-dihydro-(1H)-pyrazole-1-carboxamide (from the isocyanate derived from (−)-cis-myrtanylamine (CAS 38235-68-6))

¹H-NMR (400 MHz, CDCl₃) δ 0.85-0.94 (m, 4H), 1.15 (s, 3H), 1.20-2.40 (m, 18H), 3.20-3.39 (m, 2H), 3.99-4.07 (m, 1H), 4.22-4.30 (m, 1H), 4.37 (dd, J=12 and 7, 1H), 5.93-5.99 (m, 1H), 7.14-7.23 (m, 2H), 7.65-7.71 (m, 1H), 8.57-8.60 (m, 2H).

### Compound 16

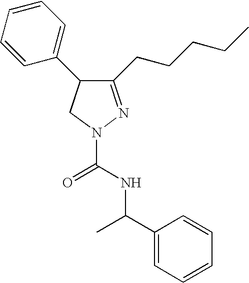

### N-(1-Phenyl-ethyl)-3-(n-pentyl)-4-phenyl-4,5-dihydro-(1H)-pyrazole-1-carboxamide

LC/MS (Method B). Retention time: 4.50 minutes: Found molecular mass (API-ES; positive scan)=364.

R_{f}(dichloromethane/methanol=99/1 (v/v))=0.25.

### Compound 17

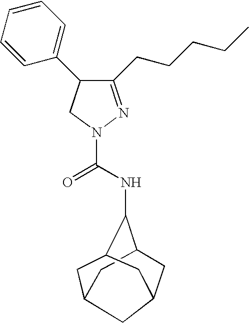

### N-(2-Adamantyl)-3-(n-pentyl)-4-phenyl-4,5-dihydro-(1H)-pyrazole-1-carboxamide

LC/MS (Method B). Retention time: 5.55 minutes: Found molecular mass (API-ES; positive scan)=394. Melting point: 71° C. R_{f}(dichloromethane/methanol=99/1 (v/v))=0.2.

### Compound 18

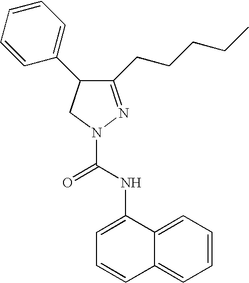

### N-(1-Naphtyl)-3-(n-pentyl)-4-phenyl-4,5-dihydro-(1H)-pyrazole-1-carboxamide

¹H-NMR (400 MHz, CDCl₃) δ 0.90 (t, J=7, 3H), 1.25-1.37 (m, 4H), 1.55-1.65 (m, 2H), 2.12-2.32 (m, 2H), 4.02 (dd, J=10 and 6, 1H), 4.26 (dd, J=12 and 6, 1H), 4.39 (t, J˜12, 1H), 7.20-7.57 (m, 8H), 7.62 (d, J=8, 1H), 7.62 (d, J=8, 1H), 7.87 (d, J=8, 1H), 7.96 (d, J=8, 1H), 8.14 (d, J=8, 1H), 8.60 (br s, 1H). R_{f}(dichloromethane/methanol=99/1 (v/v))=0.3.

### Compound 19

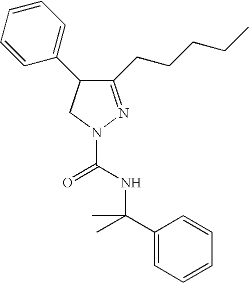

### N-(1-Methyl-1-phenyl-ethyl)-3-(n-pentyl)-4-phenyl-4,5-dihydro-(1H)-pyrazole-1-carboxamide

¹H-NMR (300 MHz, CDCl₃) δ 0.86 (t, J=7, 3H), 1.20-1.60 (m, 6H), 1.75 (s, 3H), 1.78 (s, 3H), 2.00-2.20 (m, 2H), 3.79-3.85 (m, 1H), 4.05-4.22 (m, 2H), 6.37 (br s, 1H), 7.13-7.37 (m, 8H), 7.46-7.51 (m, 2H). R_{f}(dichloromethane/methanol=99/1 (v/v))=0.2.

### Compound 20

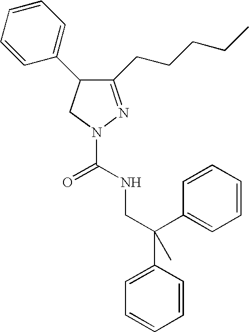

### N-(2,2-Diphenylpropyl)-3-(n-pentyl)-4-phenyl-4,5-dihydro-(1H)-pyrazole-1-carboxamide

LC/MS (Method B). Retention time: 4.99 minutes: Found molecular mass (API-ES; positive scan)=454. R_{f}(dichloromethane/methanol=99/1 (v/v))=0.3.

### Compound 21

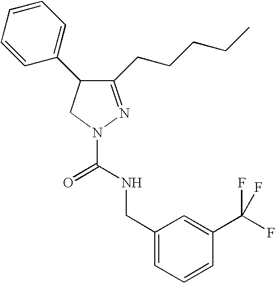

### N-((3-Trifluoromethyl)benzyl)-3-(n-pentyl)-4phenyl-4,5-dihydro-(1H)-pyrazole-1-carboxamide

LC/MS (Method B). Retention time: 4.43 minutes: Found molecular mass (API-ES; positive scan)=418.

R_{f}(dichloromethane/methanol=99/1 (v/v))=0.2.

### Compound 22

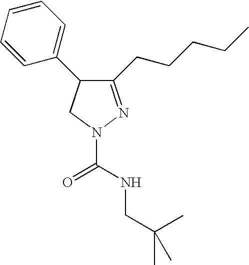

### N-(2,2-Dimethylpropyl)-3-(n-pentyl)-4-phenyl-4,5-dihydro-(1H)-pyrazole-1-5 carboxamide

LC/MS (Method B). Retention time: 4.36 minutes: Found molecular mass (API-ES; positive scan)=330.

R_{f}(dichloromethane/methanol=99/1 (v/v))=0.3.

### Compound 23

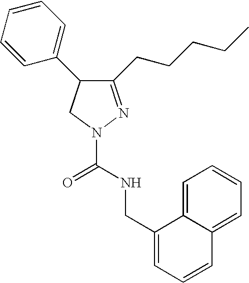

### N-(Naphthalen-1-yl-methyl)-3-(n-pentyl)-4-phenyl-4,5-dihydro-(1H)-pyrazole-1-carboxamide

LC/MS (Method B). Retention time: 6.41 minutes: Found molecular mass (API-ES; positive scan)=400.

R_{f}(dichloromethane/methanol=99/1 (v/v))=0.3.

### Compound 24

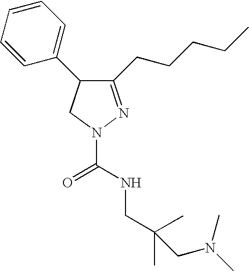

### N-[(3-Dimethylamino)-2,2-dimethylpropyl]-3-(n-pentyl)-4-phenyl-4,5-dihydro-(1H)-pyrazole-1-carboxamide

¹H-NMR (400 MHz, CDCl₃) δ 0.80-0.90 (m, 3H), 0.96 (br s, 6H), 1.20-1.28 (m, 4H), 1.46-1.57 (m, 2H), 2.00-2.16 (m, 2H), 2.24 (s, 2H), 2.32 (s, 6H), 3.15-3.27 (m, 2H), 3.87 (dd, J˜11 and 7, 1H), 4.10 (dd, J˜11 and 7, 1H), 4.23 (br t, J˜11, 1H), 7.14-7.18 (m, 2H), 7.26-7.38 (m, 4H).

### Compound 25

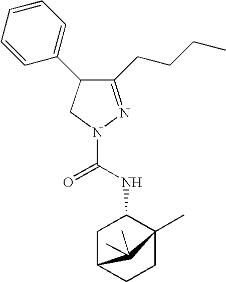

### N-[Endo-(1R,2S,4R)-1,7,7-trimethylbicyclo[2.2.1]hept-2-yl]-3-(n-butyl)-4-phenyl-4,5-dihydro-(1H)-pyrazole-1-carboxamide (from the isocyanate derived from 1R-(+)-bornylamine (CAS 32511-34-5)).

¹H-NMR (400 MHz, CDCl₃) δ 0.80-0.94 (m, 10H), 0.97 (s, 3H), 1.20-1.70 (m, 8H), 1.72-1.84 (m, 1H), 2.01-2.10 (m, 1H), 2.14-2.24 (m, 1H), 2.34-2.44 (m, 1H), 3.82-3.89 (m, 1H), 4.09-4.27 (m, 3H), 6.01 (br d, J˜9, 1 H), 7.16 (br d, J˜8, 2H), 7.26-7.37 (m, 3H).

### Compound 26

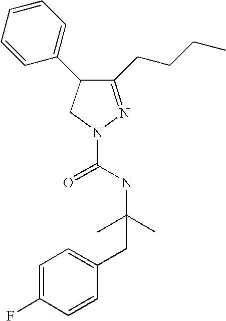

### N-(2-(4-fluorophenyl)-1,1-dimethyl-ethyl)-3-(n-butyl)-4-phenyl-4,5-dihydro-(1H)-pyrazole-1-carboxamide

¹H-NMR (400 MHz, CDCl₃) δ 0.84 (t, J=7, 3H), 1.20-1.52 (m, 10H), 1.97-2.17 (m, 2H), 3.02 (d, J=13, 1H), 3.09 (d, J=13, 1H), 3.88 (dd, J=10 and 6, 1H), 4.08-4.15 (m, 1H), 4.18-4.24 (m, 1H), 5.76 (br s, 1H), 6.93-7.01 (m, 2H), 7.12-7.18 (m, 4H), 7.26-7.38 (m, 3H).

### Compound 27

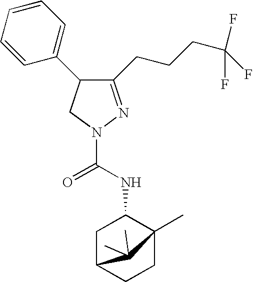

### N-[Endo-(1R,2S,4R)-1,7,7-trimethylbicyclo[2.2.1]hept-2-yl]-3-(4,4,4-trifluoro-n-butyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.84-0.94 (m, 7H), 0.97 (s, 3H), 1.20-1.29 (m, 1H), 1.36-1.47 (m, 1H), 1.53-1.63 (m, 1H), 1.67 (br t, J-4, 1H), 1.71-1.89 (m, 3H), 2.00-2.23 (m, 4H), 2.35-2.51 (m, 1H), 3.86-3.93 (m, 1H), 4.08-4.30 (m, 3H), 6.00 (br d, J˜9, 1H), 7.15 (br d, J˜8, 2H), 7.26-7.39 (m, 3H).

### Compound 28

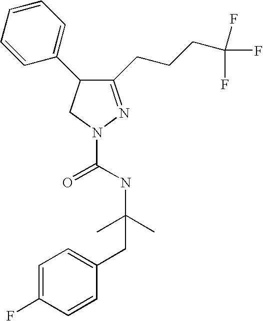

### N-(2-(4-fluorophenyl)-1,1-dimethyl-ethyl)-3-(4,4,4-trifluoro-n-butyl)-4-phenyl-4,5-dihydro-(1H)-pyrazole-1-carboxamide

¹H-NMR (400 MHz, CDCl₃) δ 1.38 (s, 3H), 1.39 (s, 3H), 1.67-1.84 (m, 2H), 1.92-2.16 (m, 4H), 3.02 (d, J=13, 1H), 3.08 (d, J =13, 1H), 3.91 (dd, J=11 and 7, 1H), 4.06-4.13 (m, 1H), 4.24 (t, J=11, 1H), 5.72 (br s, 1H), 6.94-7.00 (m, 2H), 7.12-7.18 (m, 4H), 7.28-7.40 (m, 3H).

### Compound 29

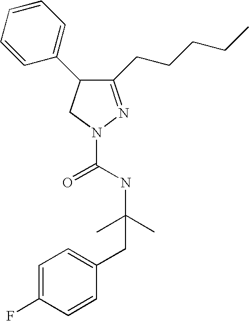

### N-(2-(4Fluorophenyl)-1,1-dimethyl-ethyl)-3-(n-pentyl)-4-phenyl-4,5-dihydro-(1H)-pyrazole-1-carboxamide

¹H-NMR (400 MHz, CDCl₃) δ 0.84 (t, J=7, 3H), 1.14-1.30 (m, 4H), 1.32-1.54 (m, 8H), 1.96-2.14 (m, 2H), 3.03 (d, J=13, 1H), 3.09 (d, J=13, 1H), 3.88 (dd, J=11 and 6, 1H), 4.08-4.14 (m, 1H), 4.21 (t, J=11, 1H), 5.76 (brs, 1H), 6.93-7.00 (m, 2H), 7.13-7.18 (m, 4H), 7.28-7.38 (m, 3H).

### Compound 30

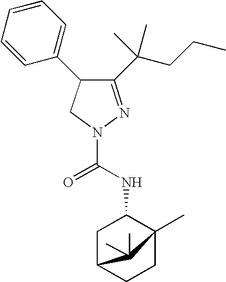

### N-[Endo-(1R,2S,4R)-1,7,7-Trimethylbicyclo[2.2.1]hept-2-yl]-3-(1,1-dimethyl-n-butyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.73-0.93 (m, 13H), 0.97 (s, 3H), 1.05 (s, 3H), 1.10-1.70 (m, 8H), 1.73-1.85 (m, 1H), 2.36-2.45 (m, 1H), 3.88-3.95 (m, 1H), 4.02-4.21 (m, 3H), 6.12 (brd, J˜9, 1H), 7.13-7.19 (m, 2H), 7.21-7.32 (m, 3H).

### Compound 31

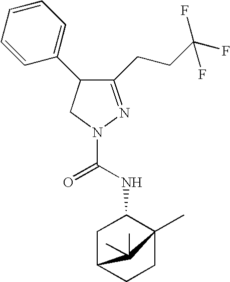

### N-[Endo-(1R,2S,4R)-1,7,7-Trimethylbicyclo[2.2.1]hept-2-yl]-3-(3,3,3-trifluoropropyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.81-0.94 (m, 7H), 0.97 (s, 3H), 1.21-1.30 (m, 1H), 1.36-1.48 (m, 1H), 1.52-1.70 (m, 2H), 1.74-1.85 (m, 1H), 2.30-2.48 (m, 5H), 3.88-3.95 (m, 1H), 4.10-4.33 (m, 3H), 5.96 (brd, J˜9, 1H), 7.17 (brd, J=8, 2H), 7.28-7.40 (m, 3H).

### Compound 32

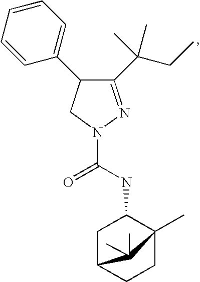

### N-[Endo-(1R,2S,4R)-1,7,7-Trimethylbicyclo[2.2.1]hept-2-yl]-3-(1,1-dimethylpropyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.77 (t, J=7, 3H), 0.81-0.95 (m, 10H), 0.97 (s, 3H), 1.04 (s, 3H), 1.10-1.70 (m, 6H), 1.74-1.85 (m, 1H), 2.34-2.46 (m, 1H), 3.88-3.94 (m, 1H), 4.02-4.20 (m, 3H), 6.13 (br d, J˜9, 1H), 7.13-7.18 (m, 2H), 7.21-7.33 (m, 3H).

### Compound 33

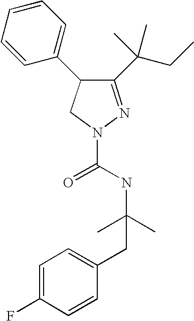

### N-(2-(4-Fluorophenyl)-1,1-dimethyl-ethyl)-3-(1,1-dimethylpropyl)-4-phenyl-4,5-dihydro-(1H)-pyrazole-1-carboxamide

¹H-NMR (400 MHz, CDCl₃) δ 0.72 (t, J=7, 3H), 0.83 (s, 3H), 0.99 (s, 3H), 1.22-1.31 (m, 2H), 1.40 (s, 6H), 2.97-3.09 (m, 2H), 3.88-3.94 (m,1H), 4.01-4.14 (m, 2H), 5.84 (br s, 1H), 6.93-7.01 (m, 2H), 7.11-7.19(m, 4H), 7.22-7.33 (m, 3H).

### Compound 34

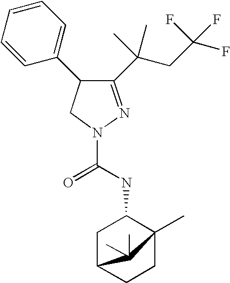

### N-[Endo-(1R,2S,4R)-1,7,7-trimethylbicyclo[2.2.1]hept-2-yl]-3-(1,1-dimethyl-3,3,3-trifluoropropyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.81-0.95 (m, 7H), 0.97 (s, 3H), 1.09-1.60 (m, 9H, including 2 Me singlets at 1.12 and 1.13 ppm ), 1.66-1.71 (m, 1H), 1.75-1.85 (m, 1H), 2.24-2.47 (m, 3H), 3.91-3.98 (m, 1H), 4.08-4.24 (m, 3H), 6.01-6.08 (m, 1H), 7.13-7.19 (m, 2H), 7.24-7.36 (m, 3H).

### Compound 35

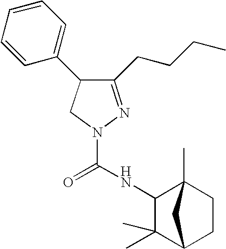

### N-[endo-(1R)-1,3,3-trimethylbicyclo[2.2.1]hept-2-yl]-3-(n-butyl)-4-phenyl-4,5-dihydro-(1H)-pyrazole-1-carboxamide (from the isocyanate derived from endo-(1R)-1,3,3-trimethylbicyclo[2.2.1]heptan-2-amine

¹H-NMR (400 MHz, CDCl₃) δ 0.78-0.89 (m, 6H), 1.05-1.78 (m, 17H), 2.01-2.22 (m, 2H), 3.56 (dd, J=10 and 2, 1H), 3.83-3.91 (m, 1H), 4.09-4.27 (m, 2H), 6.07 (br d, J˜10, 1H), 7.14-7.18 (m, 2H), 7.26-7.37 (m, 3H).

### Compound 36

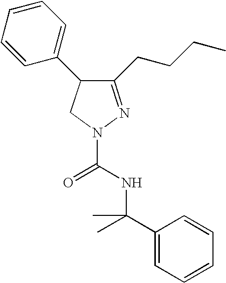

### N-(1-Methyl-1-phenyl-ethyl)-3-(n-butyl)-4-phenyl-4,5-dihydro-(1H)-pyrazole-1-carboxamide

¹H-NMR (400 MHz, CDCl₃) δ 0.86 (t, J=7, 3H), 1.21-1.33 (m, 2H), 1.38-1.54 (m, 2H), 1.75 (s, 3H), 1.77 (s, 3H), 2.04-2.22 (m, 2H), 3.82 (dd, J=9.7 and 5.6, 1H), 4.07-4.20 (m, 2H), 6.38 (br s, 1H), 7.13-7.36 (m, 8H), 7.48 (br d J˜8, 2H).

### Compound 37

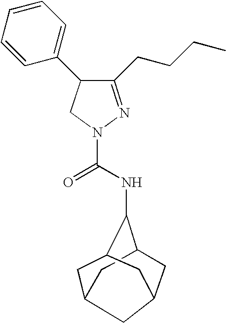

### N-(2-Adamantyl)-3-(n-butyl)-4-phenyl-4,5-dihydro-(1H)-pyrazole-1-carboxamide

¹H-NMR (400 MHz, CDCl₃) δ 0.86 (t, J=7, 3H), 1.23-1.34 (m, 2H), 1.41-1.53 (m, 2H), 1.62-2.10 (m, 15H), 2.13-2.22 (m, 1H), 3.86 (dd, J=10.5 and 6.5, 1H), 3.98-4.03 (m 1H), 4.13-4.26 (m, 2H), 6.38 (br d, J˜8, 1H), 7.16 (br d, J˜8, 2H), 7.24-7.36 (m, 3H).

### Compound 38

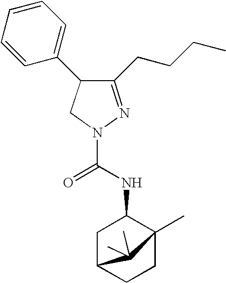

### N-[Exo-(1R,2R,4R)-1,7,7-trimethylbicyclo[2.2.1]hept-2-yl]-3-(n-butyl)-4-phenyl-4,5-dihydro-(1H)-pyrazole-1-carboxamide (from the isocyanate derived from exo-1R-bornylamine

¹H-NMR (400 MHz, CDCl₃) δ 0.79-0.92 (m, 10H), 0.98 (s, 3H), 1.12-1.77 (m, 9H), 1.86-1.93 (m, 1H), 1.99-2.19 (m, 2H), 3.80-3.90 (m, 2H), 4.07-4.25 (m, 2H), 6.06 (br d, J˜9, 1H), 7.15 (br d, J˜8, 2H), 7.26-7.37 (m, 3H).

### Compound 39

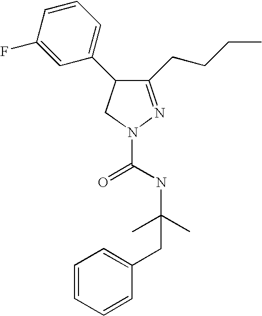

### N-(2-phenyl-1,1-dimethyl-ethyl)-3-(n-butyl)-4-(3-fluorophenyl)-4,5-dihydro-(1H)-pyrazole-1-carboxamide

¹H-NMR (400 MHz, CDCl₃) δ 0.85 (t, J=7, 3H), 1.20-1.32 (m, 2H), 1.37-1.51 (m, 8H), 1.98-2.18 (m, 2H), 3.03 (d, J=18, 1H), 3.11 (d, J=18, 1H), 3.88 (dd, J=11 and 7, 1H), 4.07-4.24 (m, 2H), 5.82 (br s, 1H), 6.84-7.04 (m, 3H), 7.17-7.36 (m, 6H).

### Compound 40

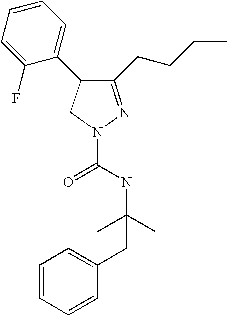

### N-(2-phenyl-1,1-dimethyl-ethyl)-3-(n-butyl)-4-(2-fluorophenyl)-4,5-dihydro-(1H)-pyrazole-1-carboxamide

¹H-NMR (400 MHz, CDCl₃) δ 0.85 (t, J=7, 3H), 1.20-1.32 (m, 2H), 1.34-1.53 (m, 8H), 2.00-2.19 (m, 2H), 3.03 (d, J=18, 1H), 3.11 (d, J=18, 1H), 3.89 (dd, J=11 and 7, 1H), 4.20 (t, J=11, 1H), 4.47 (dd, J=11 and 7, 1H), 5.81 (br s, 1H), 7.05-7.31 (m, 9H).

### Compound 41

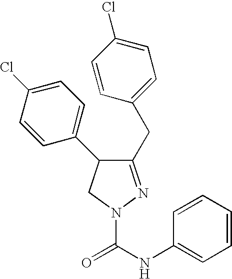

### N-Phenyl-3-(4-chlorobenzyl)-4-(4-chlorophenyl)-4,5-dihydro-(1H)-pyrazole-1-carboxamide

Melting point: 156° C.

### Compound 42

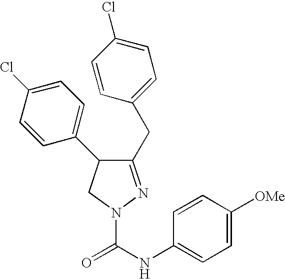

### N-(4-Methoxyphenyl)-3-(4-chlorobenzyl)-4-(4-chlorophenyl)-4,5-dihydro-(1H)-pyrazole-1-carboxamide

Melting point: 116-119° C.

### Compound 43

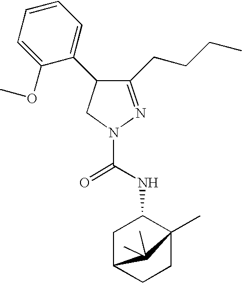

### N-[Endo-(1R,2S,4R)-1,7,7-trimethylbicyclo[2.2.1]hept-2-yl]-3-(n-butyl)-4-(2-methoxyphenyl)-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.84-0.95 (m, 10H), 0.97 (s, 3H), 1.20-1.68 (m, 8H), 1.73-1.83 (m, 1H), 2.01-2.11 (m, 1H), 2.16-2.26 (m, 1H), 2.34-2.44(m, 1H), 3.78-3.85 (m, 4H), 4.08-4.23 (m, 2H), 4.50-4.58 (m, 1H), 5.98-6.03 (m, 1H), 6.86-6.96 (m, 2H), 7.06 (dd, J=8 and 2, 1H), 7.22-7.28 (m, 1H).

### Compound 44

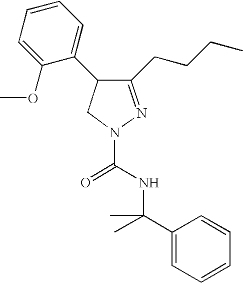

### N-(1-Methyl-1-phenyl-ethyl)-3-(n-butyl)-4-(2-methoxyphenyl)-4,5-dihydro-(1H)-pyrazole-1-carboxamide

¹H-NMR (400 MHz, CDCl₃) δ 0.85 (t, J=7, 3H), 1.24-1.56 (m, 4H), 1.75 (s, 3H), 1.76 (s, 3H), 2.01-2.11 (m, 1H), 2.16-2.25 (m, 1H), 3.75-3.82 (m, 4H), 4.07 (t, J=11, 1H), 4.53 (dd, J=11 and 7, 1H), 6.36 (br s, 1H), 6.87 (d, J=8, 1H), 6.90-6.95 (m, 1H), 7.06 (dd, J=8 and 2, 1H), 7.19-7.28 (m, 2H), 7.33 (t, J=8, 2H), 7.48 (br d, J=8, 2H).

### Compound 45

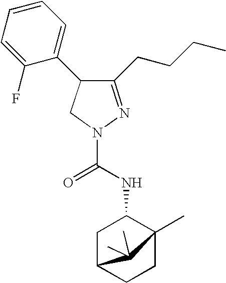

### N-[Endo-(1R,2S,4R)-1,7,7-trimethylbicyclo[2.2.1]hept-2-yl]-3-(n-butyl)-4-(2-fluorophenyl)-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.85-0.95 (m, 10H), 0.97 (s, 3H), 1.21-1.83 (m, 9H), 2.05-2.14 (m, 1H), 2.19-2.28 (m, 1H), 2.35-2.45 (m, 1H), 3.82-3.90 (m, 1H), 4.13-4.24 (m, 2H), 4.49 (dd, J=11 and 7, 1H), 6.01 (br d, J˜9, 1H), 7.03-7.18 (m, 3H), 7.23-7.30 (m, 1H).

### Compound 46

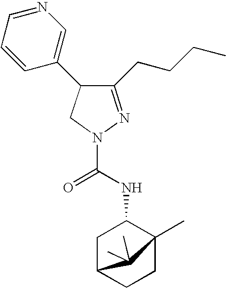

### N-[Endo-(1R,2S,4R)-1,7,7-trimethylbicyclo[2.2.1]hept-2-yl]-3-(n-butyl)-4-(pyrid-3-yl)-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.85-0.94 (m, 10H), 0.97 (s, 3H), 1.21-1.70 (m, 8H), 1.74-1.84 (m, 1H), 2.02-2.12 (m, 1H), 2.16-2.27 (m, 1H), 2.33-2.46 (m, 1H), 3.82-3.89 (m, 1H), 4.12-4.28 (m, 3H), 6.02 (br d, J˜9, 1H), 7.28-7.33 (m, 1H), 7.47-7.52 (m, 1H), 8.47 (br d, J˜2, 1H), 8.56 (dd, J=5 and 2, 1H).

### Compound 47

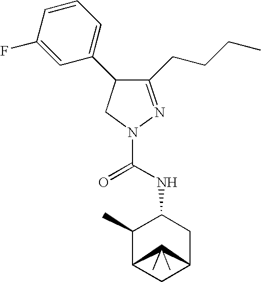

### N-[(1R,2R,3R,5S)-2,7,7-trimethylbicyclo[3.1.1]hept-3-yl]-3-(n-butyl)-4-(3-fluorophenyl)-4,5-dihydro-(1H)-pyrazole-1-carboxamide (from the isocyanate derived from (1R,²R,³R,5S)-(−)-isopinocampheylamine (CAS 69460-11-3))

¹H-NMR (400 MHz, CDCl₃) δ 0.87 (t, J=7, 3H), 0.96 (d, J=9, 1H), 1.02-2.00 (m, 14H), 2.02-2.10 (m, 1H), 2.13-2.23 (m, 1H), 2.36-2.46 (m, 2H), 2.58-2.70 (m, 2H), 3.83-3.90 (m, 1H), 3.98-4.27 (m, 4H), 5.82 (br d, J˜9, 1H), 6.85-6.90 (m, 1H), 6.94-7.01 (m, 2H), 7.27-7.34 (m, 1H).

### Compound 48

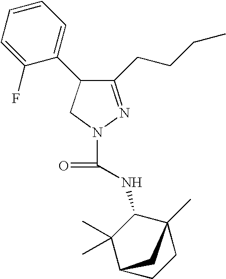

### N-[endo-(1R)-1,3,3-trimethylbicyclo[2.2.1]hept-2-yl]-3-(n-butyl)-4(2-fluorophenyl)-4,5-dihydro-(1H)-pyrazole-1-carboxamide (from the isocyanate derived from endo-(1R)-1,3,3-trimethylbicyclo[2.2.1]heptan-2-amine

¹H-NMR (400 MHz, CDCl₃) δ 0.75-0.83 (m, 6H), 1.02, 1.03, 1.04, 1.05 (4×singlet from diastereomeric CH₃ groups, 6H), 1.08-1.70 (m, 11H), 1.95-2.18 (m, 2H), 3.48 (br d, J˜10, 1H), 3.76-3.84 (m, 1H), 4.08-4.17 (m, 1H), 4.37-4.47 (m, 1H), 5.99 (brd, J˜10, 1H), 6.95-7.09 (m, 3H), 7.15-7.22 (m, 1H). 20

### Compound 49

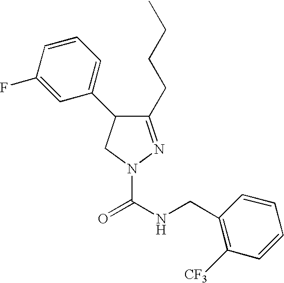

### N-[2-(trifluoromethyl)benzyl]-3-(n-butyl)-4-(3-fluorophenyl)-4,5-dihydro-(1H)-pyrazole-1-carboxamide

¹H-NMR (400 MHz, CDCl₃) δ 0.85 (t, J=7, 3H), 1.21-1.32 (m, 2H), 1.39-1.53 (m, 2H), 2.01-2.20 (m, 2H), 3.89 (dd, J=11 and 6.4, 1H), 4.09-4.15 (m, 1H), 4.23 (t, J=11, 1H), 4.70 (d, J=7, 2H), 6.36 (br t, J=7, 1H), 6.84-6.89 (m, 1H), 6.92-7.02 (m, 2H), 7.28-7.40 (m, 2H), 7.52-7.57 (m, 1H), 7.63-7.70 (m, 2H).

### Compound 50

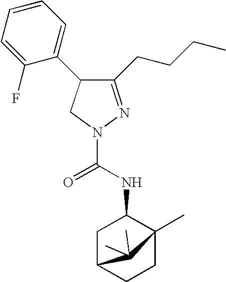

### N-[Exo-(1R,2R,4R)-1,7,7-trimethylbicyclo[2.2.1]hept-2-yl]-3-(n-butyl)-4-(2-fluorophenyl)-4,5-dihydro-(1H)-pyrazole-1-carboxamide (from the isocyanate derived from exo-1R-bornylamine

¹H-NMR (400 MHz, CDCl₃) δ 0.81-0.92 (m, 10H), 0.97 (s, 3H), 1.11-1.77 (m, 9H), 1.89 (dd, J=13 and 9, 1H), 2.03-2.22 (m, 2H), 3.80-3.90 (m, 2H), 4.13-4.23 (m, 1H), 4.43-4.51 (m, 1H), 6.06 (brd, J˜9, 1H), 7.03-7.15 (m, 3H), 7.22-7.30 (m,1H).

### Compound 51

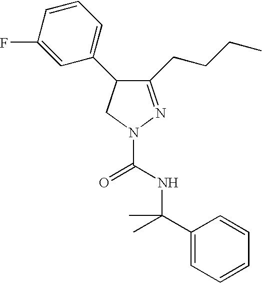

### N-(1-Methyl-1-phenyl-ethyl)-3-(n-butyl)-4-(3-fluorophenyl)-4,5-dihydro-(1H)-pyrazole-1-carboxamide

¹H-NMR (400 MHz, CDCl₃) δ 0.87 (t, J=7, 3H), 1.24-1.56 (m, 4H), 1.75 (s, 3H), 1.77 (s, 3H), 2.02-2.11 (m, 1H), 2.15-2.24 (m, 1H), 3.81 (dd, J=9.3 and 4.8 Hz, 1H), 4.07-4.19 (m, 2H), 6.36 (br s, 1H), 6.86-6.90 (m, 1H), 6.93-7.01 (m, 2H), 7.20-7.37 (m, 4H), 7.45-7.50 (m, 2H).

### Compound 52

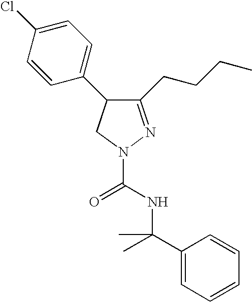

### N-(1-Methyl-1-phenyl-ethyl)-3-(n-butyl)-4-(4-chlorophenyl)-4,5-dihydro-(1H)-pyrazole-1-carboxamide

¹H-NMR (400 MHz, CDCl₃) δ 0.87 (t, J=7, 3H), 1.23-1.55 (m, 4H), 1.74 (s, 3H), 1.78 (s, 3H), 1.99-2.09 (m, 1H), 2.12-2.22 (m, 1H), 3.78 (dd, J=10 and 5.5 Hz, 1H), 4.05-4.19 (m, 2H), 6.36 (br s, 1H), 7.10 (br d, J=8, 2H), 7.20-7.37 (m, 5H), 7.48 (br d, J=8, 2H).

### Compound 53

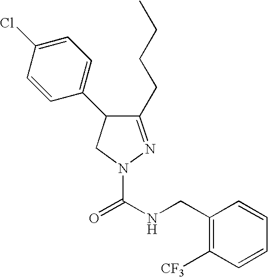

### N-[2-(trifluoromethyl)benzyl]-3-(n-butyl)-4-(4-chlorophenyl)-4,5-dihydro-(1H)-pyrazole-1-carboxamide

¹H-NMR (400 MHz, CDCl₃) δ 0.85 (t, J=7, 3H), 1.20-1.53 (m, 4H), 1.98-2.18 (m, 2H), 3.86 (dd, J=11 and 6.5, 1H), 4.08-4.15 (m, 1H), 4.23 (t, J=11, 1H), 4.69 (br d, J=6.3, 2H), 6.36 (br t, J=6.3, 1H), 7.09 (br d, J=8, 2H), 7.31 (br d, J=8, 2H), 7.35-7.41 (m,1H), 7.51-7.58 (m, 1H), 7.63-7.70 (m, 2H).

### Compound 54

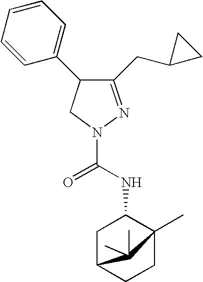

### N-[Endo-(1R,2S,4R)-1,7,7-trimethylbicyclo[2.2.1]hept-2-yl]-3-(cyclopropylmethyl)-4-phenyl-4,5-dihydro-(1H)-pyrazole-1-carboxamide (from the isocyanate derived from 1R-(+)-bornylamine (CAS 32511-34-5))

¹H-NMR (400 MHz, CDCl₃) δ-0.04-0.08 (m, 2H), 0.39-0.53 (m, 2H), 0.75-0.94 (m, 8H), 0.97 (s, 3H), 1.21-1.29 (m, 1H), 1.35-1.46 (m, 1H), 1.57-1.69 (m, 2H), 1.74-1.84 (m, 1H), 1.90-1.98 (m, 1H), 2.15-2.24 (m, 1H), 2.33-2.44 (m, 1H), 3.83-3.89 (m, 1H), 4.12-4.33 (m, 3H), 6.02-6.09 (m, 1H), 7.16 (br d, J=8, 2H), 7.25-7.37 (m, 3H).

### Compound 55

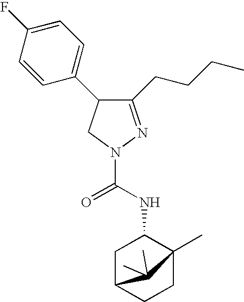

### N-[Endo-(1R,2S,4R)-1,7,7-trimethylbicyclo[2.2.1]hept-2-yl]-3-(n-butyl)4-(4-fluorophenyl)-4,5-dihydro-(1H)-pyrazole-1-carboxamide (from the isocyanate derived from 1R-(+)-bornylamine (CAS 32511-34-5))

¹H-NMR (400 MHz, CDCl₃) δ 0.85-0.95 (m, 10H), 0.97 (s, 3H), 1.20-1.69 (m, 9H), 1.73-1.85 (m, 1H), 2.01-2.10 (m, 1H), 2.14-2.24 (m, 1H), 2.34-2.45 (m, 1H), 3.79-3.86 (m, 1H), 4.08-4.25 (m, 2H), 6.01 (br d, J˜9, 1H), 7.00-7.06 (m, 2H), 7.11-7.16 (m, 2H).

### Compound 56

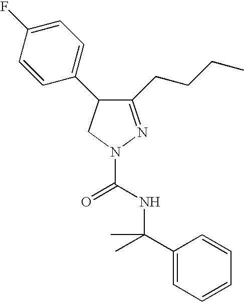

### N-(1-Methyl-1-phenyl-ethyl)-3-(n-butyl)-4-(4-fluorophenyl)-4,5-dihydro-(1H)-pyrazole-1-carboxamide

¹H-NMR (400 MHz, CDCl₃) δ 0.85 (t, J=7, 3H), 1.22-1.54 (m, 4H), 1.75 (s, 3H), 1.77 (s, 3H), 2.00-2.09 (m, 1H), 2.13-2.22 (m, 1H), 3.78 (dd, J=9 and 5.5, 1H), 4.07-4.18 (m, 2H), 6.36 (br s, 1H), 7.00-7.06 (m, 2H), 7.10-7.16 (m, 2H), 7.20-7.25 (m, 1H), 7.32-7.37 (m, 2H), 7.46-7.50 (m, 2H).

### Compound 57

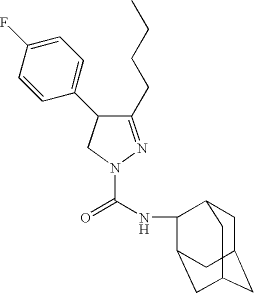

### N-(Adamant-2-yl)-3-(n-butyl)-4-(4-fluorophenyl)-4,5-dihydro-(1H)-pyrazole-1-carboxamide

¹H-NMR (400 MHz, CDCl₃) δ 0.85 (t, J=7, 3H), 1.22-1.54 (m, 4H), 1.62-2.09 (m, 15H), 2.13-2.22 (m, 1H), 3.82 (dd, J=10 and 6, 1H), 3.97-4.03 (m, 1H), 4.08-4.23 (m, 2H), 6.37 (br d, J=9, 1H), 7.00-7.06 (m, 2H), 7.10-7.16 (m, 2H).

### Compound 58

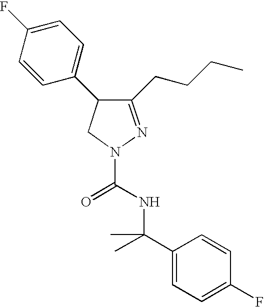

### N-(1-Methyl-1-(4-fluorophenyl)-ethyl)-3-(n-butyl)-4-(4-fluorophenyl)-4,5-dihydro-(1H)-pyrazole-1-carboxamide

¹H-NMR (400 MHz, CDCl₃) δ 0.87 (t, J=7, 3H), 1.21-1.56 (m, 4H), 1.72 (s, 3H), 1.75 (s, 3H), 2.00-2.22 (m, 2H), 3.74-3.78 (m, 1H), 4.07-4.17 (m, 2H), 6.34 (br s, 1H), 6.98-7.06 (m, 4H), 7.09-7.15 (m, 2H), 7.40-7.46 (m, 2H).

LC/MS (Method D). Retention time: 2.09 min; Found molecular mass=400.

### Compound 59

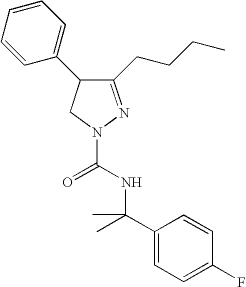

### N-(1-Methyl-1-(4-fluorophenyl)-ethyl)-3-(n-butyl)-4-phenyl-4,5-dihydro-(1H)-pyrazole-1-carboxamide

¹H-NMR (400 MHz, CDCl₃) δ 0.87 (t, J=7, 3H), 1.22-1.35 (m, 2H), 1.38-1.57 (m, 2H), 1.72 (s, 3H), 1.75 (s, 3H), 2.01-2.22 (m, 2H), 3.78-3.82 (m, 1H), 4.09-4.19 (m, 2H), 6.35 (br s, 1H), 6.98-7.04 (m, 2H), 7.13-7.17 (m, 2H), 7.25-7.37 (m, 3H), 7.41-7.47 (m, 2H).

LC/MS (Method D). Retention time: 2.05 min; Found molecular mass=382.

### Compound 60

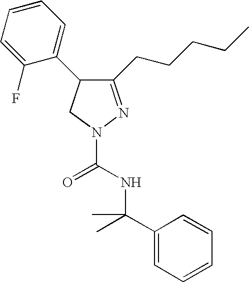

### N-(1-Methyl-1-phenyl-ethyl)-3-(n-pentyl)-4-(2-fluorophenyl)-4,5-dihydro-(1H)-pyrazole-1-carboxamide

LC/MS (Method D). Retention time: 2.13 min; Found molecular mass=396.

### Compound 61

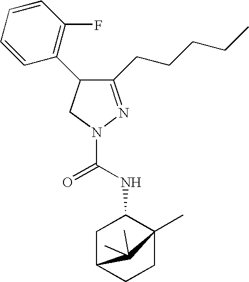

### N-[Endo-(1R,2S,4R)-1,7,7-trimethylbicyclo[2.2.1]hept-2-yl]-3-(n-pentyl)-4-(2-fluorophenyl)-4,5-dihydro-(1H)-pyrazole-1-carboxamide (from the isocyanate derived from 1R-(+)-bornylamine (CAS 32511-34-5))

LC/MS (Method D). Retention time: 2.33 min; Found molecular mass=414.

### Compound 62

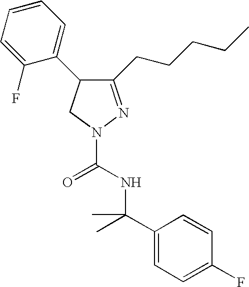

### N-(1-Methyl-1-(4fluorophenyl)-ethyl)-3-(n-pentyl)-4-(2-fluorophenyl)-4,5-dihydro-(1H)-pyrazole-1-carboxamide

LC/MS (Method D). Retention time: 2.12 min; Found molecular mass=414.

### Compound 63

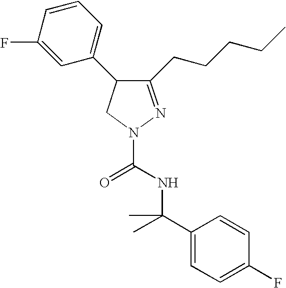

### N-(1-Methyl-1-(4-fluorophenyl)-ethyl)-3-(n-pentyl)-4-(3-fluorophenyl)-4,5-dihydro-(1H)-pyrazole-1-carboxamide

LC/MS (Method D). Retention time: 2.12 min; Found molecular mass=414.

### Compound 64

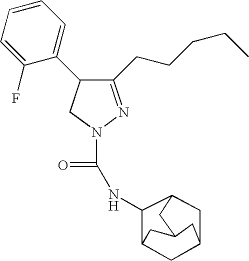

### N-(adamant-2-yl)-3-(n-pentyl)-4-(2-fluorophenyl)-4,5-dihydro-(1H)-pyrazole-1-carboxamide

LC/MS (Method D). Retention time: 2.36 min; Found molecular mass=412.

### Compound 65

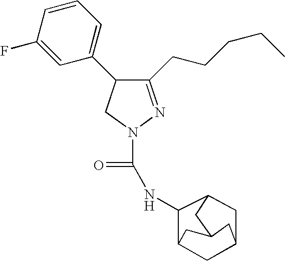

### N-(adamant-2-yl)-3-(n-pentyl)-4-(3-fluorophenyl)-4,5-dihydro-(1H)-pyrazole-1-carboxamide

LC/MS (Method D). Retention time: 2.36 min; Found molecular mass=412.

### Compound 66

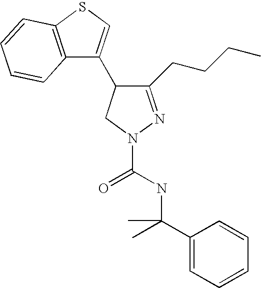

### N-(1-Methyl-1-phenyl-ethyl)-3-(n-butyl)-4-(benzo[b]thiophen-3-yl)-4,5-dihydro-(1H)-pyrazole-1-carboxamide

LC/MS (Method D). Retention time: min; Found molecular mass=419.

### Compound 67

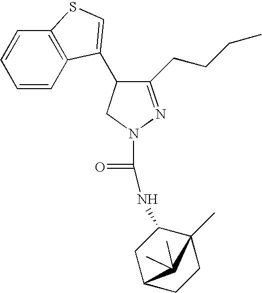

### N-[Endo-(1R,2S,4R)-1,7,7-trimethylbicyclo[2.2.1]hept-2-yl]-3-(n-butyl)-4-(benzo[b]thiophen-3-yl)-4,5-dihydro-(1H)-pyrazole-1-carboxamide (from the isocyanate derived from 1R-(+)-bornylamine (CAS 32511-34-5))

LC/MS (Method D). Retention time: 2.34 min; Found molecular mass=438.

### Compound 68

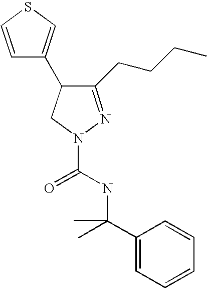

### N-(1-Methyl-1-phenyl-ethyl)-3-(n-butyl)-4-(thiophen-3-yl)-4,5-dihydro-(1H)-pyrazole-1-carboxamide

¹H-NMR (400 MHz, CDCl₃) δ 0.85 (t, J=7, 3H), 1.21-1.57 (m, 4H), 1.74 (s, 3H), 1.77 (s, 3H), 2.05-2.25 (m, 2H), 3.79 (dd, J -11 and 7, 1H), 4.08-4.13 (m, 1H), 4.28 (dd, J˜11 and 7, 1H) 6.36 (br s, 1H), 6.91 (dd, J=6 and 2, 1H), 7.06-7.08 (m, 1H), 7.19-7.24 (m, 1H), 7.30-7.37 (m, 3H), 7.45-7.49 (m, 2H).

LC/MS (Method D). Retention time: 2.00 min; Found molecular mass=370.

### Compound 69

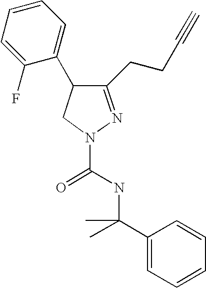

### N-(1-Methyl-1-phenyl-ethyl)-3-(but-3-ynyl)-4(2-fluorophenyl)-4,5-dihydro-(1H)-pyrazole-1-carboxamide

LC/MS (Method D). Retention time: 1.80 min; Found molecular mass=378.

### Compound 70

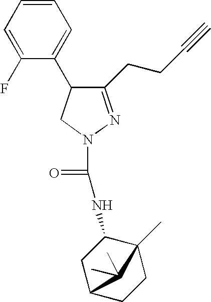

### N-[Endo-(1R,2S,4R)-1,7,7-trimethylbicyclo[2.2.1]hept-2-yl]-3-(but-3-ynyl)-4-(2-fluorophenyl)-4,5-dihydro-(1H)-pyrazole-1-carboxamide (from the isocyanate derived from 1R-(+)-bornylamine (CAS 32511-34-5))

LC/MS (Method D). Retention time: 1.99 min; Found molecular mass=396.

### Compound 71

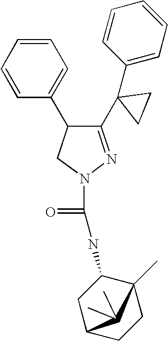

### N-[Endo-(1R,2S,4R)-1,7,7-trimethylbicyclo[2.2.1]hept-2-yl]-3-(1-phenylcyclopropyl)-4-phenyl-4,5-dihydro-(1H)-pyrazole-1-carboxamide (from the isocyanate derived from 1R-(+)-bornylamine (CAS 32511-34-5))

LC/MS (Method D). Retention time: 2.27 min; Found molecular mass=442.

### Compound 72

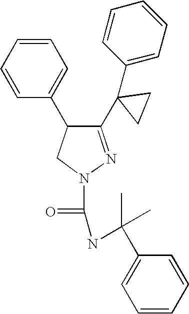

### N-(1-Methyl-1-phenyl-ethyl)-3-(1-phenylcyclopropyl)-4-phenyl-4,5-dihydro-(1H)-pyrazole-1-carboxamide

LC/MS (Method D). Retention time: 2.10 min; Found molecular mass=424.

### Compound 73

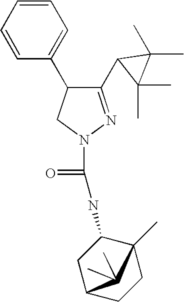

### N-[Endo-(1R,2S,4R)-1,7,7-trimethylbicyclo[2.2.1]hept-2-yl]-3-(2,2,3,3-tetramethylcyclopropyl)-4-phenyl-4,5-dihydro-(1H)-pyrazole-1-carboxamide (from the isocyanate derived from 1R-(+)-bornylamine (CAS 32511-34-5))

LC/MS (Method D). Retention time: 2.46 min; Found molecular mass=422.

### Compound 74

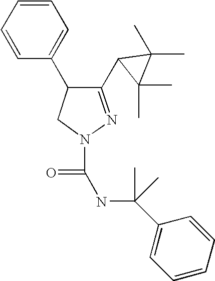

### N-(1-Methyl-1-phenyl-ethyl)-3-(2,2,3,3-tetramethylcyclopropyl)-4-phenyl-4,5-dihydro-(1H)-pyrazole-1-carboxamide

LC/MS (Method D). Retention time: 2.21 min; Found molecular mass=404.

### Compound 75

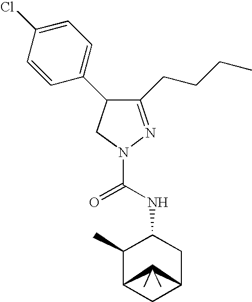

### N-[(1R,2R,3R,5S)-2,7,7-trimethylbicyclo[3.1.1]hept-3-yl]-3-(n-butyl)-4-(4-chlorophenyl)-4,5-dihydro-(1H)-pyrazole-1-carboxamide (from the isocyanate derived from (1R,2R,3R,5S)-(−)-isopinocampheylamine (CAS 69460-11-3))

LC/MS (Method D). Retention time: 2.37 min; Found molecular mass=416.

### Compound 76

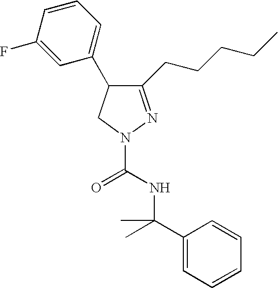

### N-(1-Methyl-1-phenyl-ethyl)-3-(n-pentyl)-4-(3-fluorophenyl)-4,5-dihydro-(1H)-pyrazole-1-carboxamide

LC/MS (Method D). Retention time: 2.15 min; Found molecular mass=396.

### Compound 77

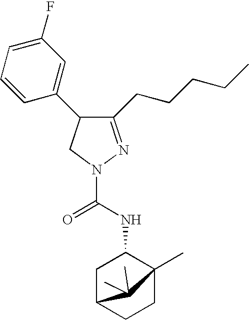

### N-[Endo-(1R,2S,4R)-1,7,7-trimethylbicyclo[2.2.1]hept-2-yl]-3-(n-pentyl)-4-(3-fluorophenyl)-4,5-dihydro-(1H)-pyrazole-1-carboxamide (from the isocyanate derived from 1R-(+)-bornylamine (CAS 32511-34-5))

LC/MS (Method D). Retention time: 2.32 min; Found molecular mass=414.

### Compound 78

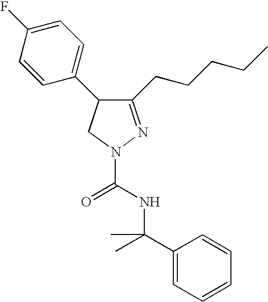

### N-(1-Methyl-1-phenyl-ethyl)-3-(n-pentyl)-4-(4-fluorophenyl)-4,5-dihydro-(1H)-pyrazole-1-carboxamide

LC/MS (Method D). Retention time: 2.07 min; Found molecular mass=396.

### Compound 79

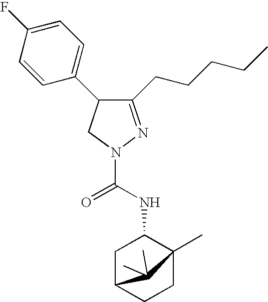

### N-[Endo-(1R,2S,4R)-1,7,7-trimethylbicyclo[2.2.1]hept-2-yl]-3-(n-pentyl)-4-(4-fluorophenyl)-4,5-dihydro-(1H)-pyrazole-1-carboxamide (from the isocyanate derived from 1R-(+)-bornylamine (CAS 32511-34-5))

LC/MS (Method D). Retention time: 2.31 min; Found molecular mass=414.

### Compound 80

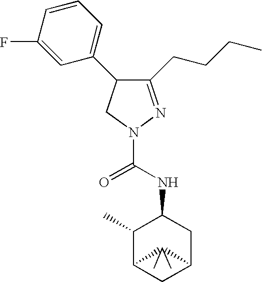

### N-[(1S,2S,3S,5R)-2,7,7-trimethylbicyclo[3.1.1]hept-3-yl]-3-(n-butyl)-4-(3-fluorophenyl)-4,5-dihydro-(1H)-pyrazole-1-carboxamide (from the isocyanate derived from (1S,2S,3S,5R)-(+)-isopinocampheylamine

LC/MS (Method D). Retention time: 2.23 min; Found molecular mass=400.

### Compound 81

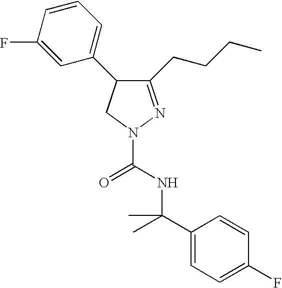

### N-(1-Methyl-1-(4-fluorophenyl)-ethyl)-3-(n-butyl)-4-(3-fluorophenyl)-4,5-dihydro-(1H)-pyrazole-1-carboxamide

LC/MS (Method D). Retention time: 2.08 min; Found molecular mass=400.

### Compound 82

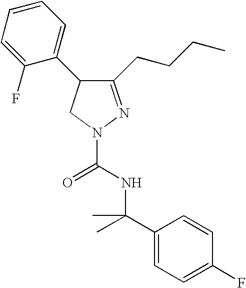

### N-(1-Methyl-1-(4-fluorophenyl)-ethyl)-3-(n-butyl)-4-(2-fluorophenyl)-4,5-dihydro-(1H)-pyrazole-1-carboxamide

LC/MS (Method D). Retention time: 2.05 min; Found molecular mass=400.

### Compound 83

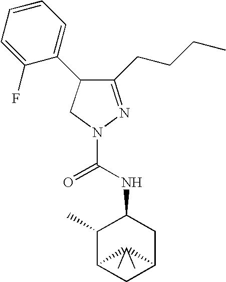

### N-[(1S,2S,3S,5R)-2,7,7-trimethylbicyclo[3.1.1]hept-3-yl]-3-(n-butyl)-4-(2-fluorophenyl)-4,5-dihydro-(1H)-pyrazole-1-carboxamide (from the isocyanate derived from (1S,2S,3S,5R)-(+)-isopinocampheylamine

LC/MS (Method D). Retention time: 2.23 min; Found molecular mass=400.

### Compound 84

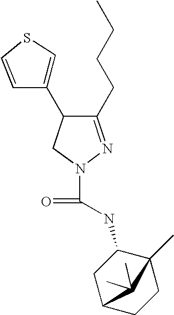

### N-[Endo-(1R,2S,4R)-1,7,7-trimethylbicyclo[2.2.1]hept-2-yl]-3-(n-butyl)-4-(thien-3-yl)-4,5-dihydro-(1H)-pyrazole-1-carboxamide (from the isocyanate derived from 1R-(+)-bornylamine (CAS 32511-34-5))

LC/MS (Method D). Retention time: 2.21 min; Found molecular mass=387.

### Compound 85

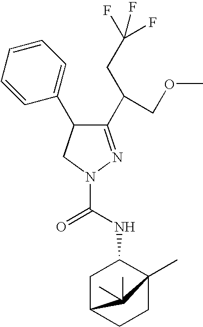

### N-[Endo-(1R,2S,4R)-1,7,7-trimethylbicyclo[2.2.1]hept-2-yl]-3-(3,3,3-trifluoro-1-methoxymethyl-propyl)-4-phenyl-4,5-dihydro-(1H)-pyrazole-1-carboxamide, mixture of diastereomer A and diastereomer B

Part A: 6,6,6-Trifluoro-4-methoxymethyl-2-phenyl-hex-1-en-3-one (Intermediate III-2) was converted with hydrazine hydrate to 3-(3,3,3-trifluoro-1-methoxymethyl-propyl)-4-phenyl-4,5-dihydro-(1H)-pyrazole (Intermediate IV-2) analogously to the procedure described for the synthesis of intermediate IV-1.

Part B: 3-(3,3,3-trifluoro-1-methoxymethyl-propyl)-4-phenyl-4,5-dihydro-(1H)-pyrazole was converted to N-[(1R,2S,5R)-rel-6,6-dimethylbicyclo[3.1.1]heptan-2-methyl]-3-(3,3,3-trifluoro-1-methoxymethyl-propyl)-4-phenyl-4,5-dihydro-(1H)-pyrazole-1-carboxamide analogously to the procedure described for the synthesis of compound 13 (via reaction with the isocyanate derived from 1R-(+)-bornylamine (CAS 32511-34-5)). This reaction gave a mixture of diastereoisomers. A mixture containing diastereomer A and diastereomer B was obtained via Sepacore chromatographic purification (petroleum ether/diethyl ether=1/1 (v/v)). R_{f} (diastereomer A)=0.15, R_{f} (diastereomer B)=0.20. ¹H-NMR (400 MHz, CDCl₃); Mixture containing diastereomer A and diastereomer B: δ 0.82-0.94 (m, 7H), 0.97 (s, 3H), 1.08-1.61 (m, 3H), 1.68 (br t, J=4.5, 1H), 1.74-1.84 (m, 1H), 2.23-2.49 (m, 3H), 2.78-2.85 (m, 1H), 3.13 and 3.15 (2xs, (OCH₃ signals, 3H), 3.17-3.35 (m, 2H), 3.93-3.98 (m, 1H), 4.13-4.28 (m, 3H), 5.93 (br d, J˜9, 1H), 7.19 (br d, J˜8, 2H), 7.28-7.38 (m, 3H).

### Compound 86

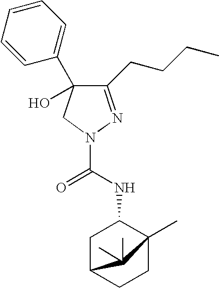

### N-[Endo-(1R,2S,4R)-1,7,7-trimethylbicyclo[2.2.1]hept-2-yl]-3-(n-butyl)-4-hydroxy-4-phenyl-4,5-dihydro-(1H)-pyrazole-1-carboxamide

Part A: 1-Phenylhexan-2-one was reacted in methanol with piperidine and acetic acid, followed by a formaldehyde solution (35% solution in water) and the resulting mixture was stirred at 55° C. for 60 hours analogously to the procedure described for the synthesis of intermediate III-1 to give 2-phenyl-hept-1-en-3-one (intermediate III-3) in 70% yield. ¹H-NMR (400 MHz, CDCl₃) δ 0.91 (t, J=7, 3H), 1.29-1.40 (m, 2H), 1.59-1.69 (m, 2H), 2.72 (t, J=7, 2H), 5.87 (s, 1H), 6.09 (s,1H), 7.28-7.40 (m, 5H).

Part B: To a mixture of 2-phenyl-hept-1-en-3-one (3.76 g, 0.02 mol), 12 ml H₂O₂ (37% aqueous solution) in 20 ml methanol is slowly added a mixture of 2 ml water and 1 ml concentrated aqueous NaOH (Cf. EP0114487). The resulting mixture is cooled to room temperature and stirred for 16 hours. The mixture is poured into water and extracted twice with diethyl ether. The diethyl ether layers were combined and filtered over hyflo and successively washed with water, aqueous acetic acid solution and brine. The resulting solution is dried over Na₂SO₄, filtered and concentrated to give 2.79 gram impure product. Flash chromatography (petroleum ether/ethyl ether=49/1 (v/v) of the crude product gave 1.31 g 1-(2-phenyloxiranyl)-pentan-1-one (Intermediate V-1) as an oil in 32% yield. ¹H-NMR (400 MHz, CDCl₃) δ 0.88 (t, J=7, 3H), 1.23-1.35 (m, 2H), 1.47-1.63 (m, 2H), 2.40-2.61 (m, 2H), 3.02 (d, J=6, 1H), 3.24 (d, J=6, 1H), 7.32-7.40 (m, 3H), 7.45-7.50 (M, 2H).

Part C: 1-(2-Phenyloxiranyl)-pentan-1-one was converted with hydrazine hydrate to 3-(n-butyl-4-hydroxy-4-phenyl-4,5-dihydro-(1H)-pyrazole (Intermediate IV-3) analogously to the procedure described for the synthesis of intermediate IV-1.

Part E: 3-(n-Butyl)-4-hydroxy-4-phenyl-4,5-dihydro-(1H)-pyrazole was converted to N-[(1R,2S,5R)-rel-6,6-dimethylbicyclo[3.1.1]heptan-2-methyl]-3-(n-butyl)-4-hydroxy-4-phenyl-4,5-dihydro-(1H)-pyrazole-1-carboxamide analogously to the procedure described for the synthesis of compound 13 (via reaction with the isocyanate derived from 1R-(+)-bornylamine (CAS 32511-34-5)). ¹H-NMR (400 MHz, CDCl₃) δ 0.81-0.94 (m, 10H), 0.96 (s, 3H), 1.21-1.32 (m, 3H), 1.35-1.70 (m, 5H), 1.74-1.86 (m, 1H), 2.01-2.11 (m, 1H), 2.15-2.28 (m, 1H), 2.32-2.45 (m, 1H), 3.10 and 3.65 (2× br s, OH, 1H), 4.01-4.20 (m, 3H), 6.06-6.14 (m, 1H), 7.27-7.43 (m, 5H).

### Compound 87

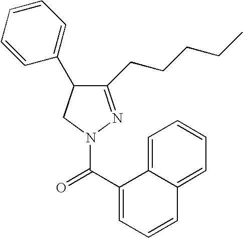

### 1-(1-Naphtoyl)-3-(n-pentyl)-4-phenyl-4,5-dihydro-(1H)-pyrazole

3-(n-Pentyl4-phenyl-4,5-dihydropyrazole (0.75 gram, 3.47 mmol) was dissolved in toluene (10 ml) and treated with 1-naphtoyl chloride (0.522 ml, 3.47 mmol) and the resulting solution was stirred at room temperature for 16 hours. The solution was concentrated, followed by flash chromatographic purification (heptane/ethylacetate=6:1 (v/v)) to give 1-(1-naphtoyl)-3-(n-pentyl)-4-phenyl-4,5-dihydro-(1H)-pyrazole (690 mg) as an oil.

LC/MS (Method B). Retention time: 5.87 minutes: Found molecular mass (API-ES; positive scan)=371. Mobile phase gradient: 0-5 minutes: Solution A/Solution B=30/70 (v/v)). >5 minutes: Solution B.

R_{f}(dichloromethane/methanol=99/1 (v/v))=0.35.

¹H-NMR (400 MHz, CDCl₃) δ 0.80-0.90 (m, 3H), 1.02-1.40 (m, 6H), 1.92-2.11 (m, 2H), 4.21-4.30 (m, 2H), 4.57-4.65 (m, 1H), 7.20 (d, J=8, 2H), 7.29-7.55 (m, 6H), 7.66 (d, J=8, 1H), 7.84-7.94 (m, 2H), 8.03 (br d, J=8, 1H).

Analogously were Prepared Compounds 88-94:

### Compound 88

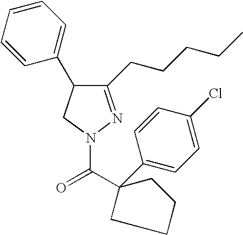

### [3-(n-pentyl)-4-phenyl-4,5-dihydro-(1H)-pyrazol-1-yl]-[1-(4-chlorophenyl)cyclopentyl]methanone

LC/MS (Method C). Retention time: 4.05 min; Found molecular mass=423.

### Compound 89

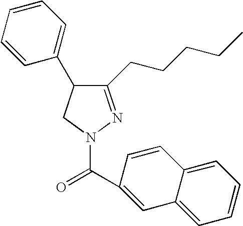

### [3-(n-pentyl)-4-phenyl-4,5-dihydro-(1H)-pyrazol-1-yl]-(napht-2-yl)methanone

LC/MS (Method C). Retention time: 3.52 min; Found molecular mass=371.

### Compound 90

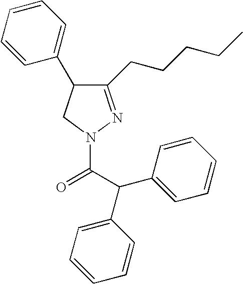

### [3-(n-pentyl)-4-phenyl-4,5-dihydro-(1H)-pyrazol-1-yl]-(diphenylmethyl)methanone

LC/MS (Method C). Retention time: 3.81 min; Found molecular mass=411.

### Compound 91

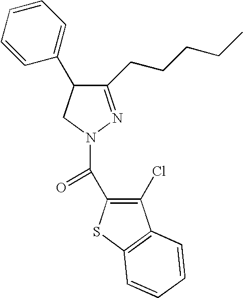

### [3-(n-pentyl)-4-phenyl-4,5-dihydro-(1H)-pyrazol-1-yl]-(3-chlorobenzothien-2-yl]methanone

LC/MS (Method C). Retention time: 3.77 min; Found molecular mass=411.

### Compound 92

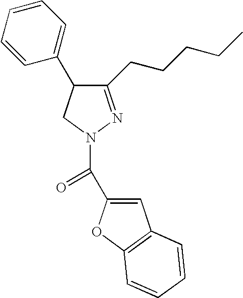

### [3-(n-pentyl)-4-phenyl-4,5-dihydro-(1H)-pyrazol-1-yl]-(benzofuran-2-yl]methanone

LC/MS (Method C). Retention time: 3.48 min; Found molecular mass=361.

### Compound 93

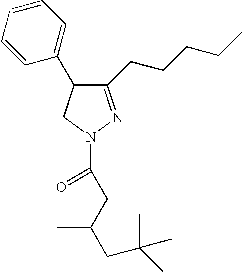

### [3-(n-pentyl)-4-phenyl-4,5-dihydro-(1H)-pyrazol-1-yl]-[2,4,4-(trimethyl)pentyl]methanone

LC/MS (Method C). Retention time: 3.98 min; Found molecular mass=357.

### Compound 94

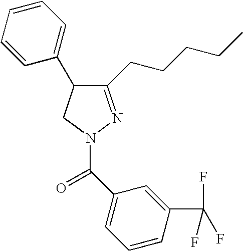

### [3-(n-pentyl)-4-phenyl-4,5-dihydro-(1H)-pyrazol-1-yl]-[3-(trifluoromethyl)phenyl]methanone

¹H-NMR (400 MHz, CDCl₃) δ 0.85 (t, J=7, 3H), 1.19-1.30 (m, 4H), 1.44-1.60 (m, 2H), 2.05-2.23 (m, 2H), 4.10-4.25 (m, 2H), 4.51 (t, J=11, 1H), 7.15-7.20 (m, 2H), 7.29-7.41 (m, 3H), 7.54-7.59 (m, 1H), 7.73 (d, J=8, 1H), 8.18 (d, J=8, 1H), 8.33 (br s, 1H).

### Compound 95

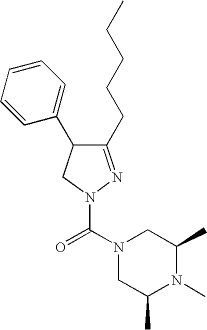

### (Cis-3,4,5-trimethylpiperazin-1-yl)[3-(n-pentyl)-4-phenyl-4,5-dihydro-(1H)-pyrazol-1-yl]methanone

Part A: To a magnetically stirred solution of N-(tert-butoxycarbonyl)-cis-3,5-dimethylpiperazine (19.7 gram, 90 mmol) in 1,4-dioxane (400 ml) was successively added a mixture of NaOH (230 ml of a 2N solution, 460 mmol) and phosphorous acid (230 ml of a 2M solution in 230 ml water, 460 mmol) followed by formaldehyde (110 ml, 37% solution in water, 1.46 mol) and the resulting mixture was reacted for 3.5 hours at 63° C. The reaction mixture was allowed to attain room temperature and extracted twice with dichloromethane. The organic layers were collected and washed with water and brine respectively and subsequently dried over Na₂SO₄, filtered and concentrated to give crude N-tert-butoxycarbonyl-cis-3,4,5-trimethylpiperazine (12 gram).

Part B: To a magnetically stirred solution of the crude N-tert-butoxycarbonyl-cis-3,4,5-trimethylpiperazine (12 gram, ˜53 mmol) in dichloromethane (180 ml) was added excess trifluoroacetic acid (40 ml) and the resulting mixture was stirred at room temperature overnight. Aqueous NaOH was added and the reaction mixture was twice extracted with dichloromethane (2×100 ml). The organic layers were collected, dried over Na₂SO₄, filtered and concentrated to give cis-3,4,5-trimethylpiperazine (3.44 gram, ˜30% yield). ¹H-NMR (400 MHz, CDCl₃) δ 1.05 (d, J=6, 6H), 1.65 (br s, 1H), 2.03-2.13 (m, 2H), 2.27 (s, 3H), 2.53 (d, J˜10, 1H), 2.57 (d, J˜10, 1H), 2.82-2.88 (m, 2H).

Part C: To a magnetically stirred solution of cis-3,4,5-trimethylpiperazine (1.5, gram, 12.7 mmol) in toluene (25 ml) was added phosgene (8 ml of a 20% solution in toluene, 15 mmol) and triethylamine (1.7 ml) and a catalytic amount of dimethylaminopyridine (DMAP). The resulting solution was stirred for 10 minutes at room temperature and 3-(n-pentyl)-4-phenyl-4,5-dihydropyrazole (2.5 gram, 12 mmol) was added and the resulting mixture was stirred at room temperature for 16 hours. The mixture was then concentrated in vacuo, followed by flash chromatographic purification (dichloromethane/7M NH₃ in methanol=97.5/2.5 (v/v)) to give (cis-3,4,5-trimethylpiperazin-1-yl)[3-(n-pentyl)-4-phenyl-4,5-dihydro-(1H)-pyrazol-1-yl]methanone (compound 26) (1.9 gram) as an oil. ¹H-NMR (300 MHz, CDCl₃) δ 0.81-0.87 (m, 3H), 1.11 (d, J=6, 6H), 1.21-1.26 (m, 4H), 1.44-1.50 (m, 2H), 2.00-2.30 (m, 7H), 2.71-2.82 (m, 2H), 3.82 (dd, J˜11 and 7, 1H), 3.97 (dd, J˜11 and 7, 1H), 4.13-4.23 (m, 3H), 7.14-7.18 (m, 2H), 7.25-7.36 (m, 3H).

### Compounds 96 and 97

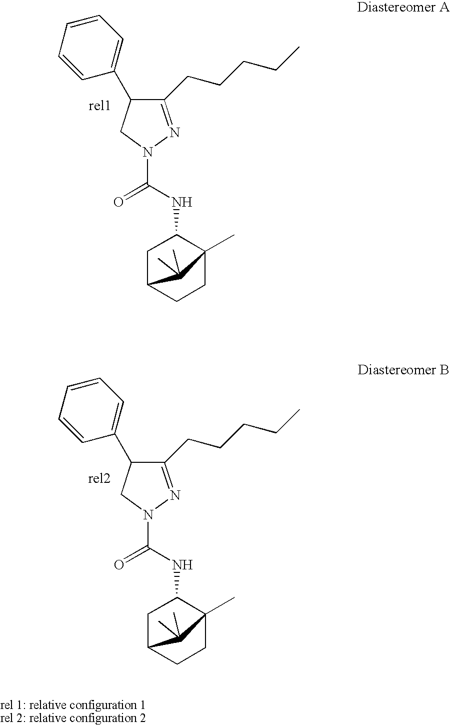

### N-Endo-[(1R,2S,4R)-1,7,7-trimethylbicyclo[2.2.1]hept-2-yl]-3-(n-pentyl)-4-phenyl-4,5-dihydro-(1H)-pyrazole-1-carboxamide (compound 27, diastereomer A) and N-Endo-[(1R,2S,4R)-1,7,7-trimethylbicyclo[2.2.1]hept-2-yl]-3-(n-pentyl)-4-phenyl-4,5-dihydro-(1H)-pyrazole-1-carboxamide (compound 28, diastereomer B)

Preparative HPLC separation of compound 13 gave compounds 96 and 97 respectively. Preparative HPLC separation procedure: A prepHPLC column LC80 (internal diameter: 8 cm) was packed with 800 grams of Chiralpak AD, 20 μ. Aceton/methanol (95/5 (v/v)) was used as the mobile phase. UV detection 220 nm. Flowrate: 2 ml/minute. Compound 96: Optical rotation ([α]_{D})=+124 (c=1.3, MeOH). ¹H-NMR (400 MHz, CDCl₃) δ 0.80-0.92 (m, 10H), 0.97 (s, 3H), 1.20-1.69 (m, 10H), 1.74-1.83 (m, 1H), 2.00-2.22 (m, 2H), 2.33-2.45 (m, 1H), 3.83-3.89 (m, 1H), 4.09-4.27 (m, 3H), 6.02 (br d, J˜10, 1H), 7.16 (br d, J˜8, 2H), 7.27-7.37 (m, 3H). ¹³C-NMR (100 MHz, CDCl₃) δ 13.74, 13.93, 18.74, 20.00, 22.32, 25.76, 28.05, 28.27, 28.45, 31.35, 38.20, 44.97, 47.99, 49.29, 53.30, 53.58, 54.42, 127.54, 127.64, 129.05, 139.67, 155.87, 158.88.

Compound 97: Optical rotation ([α]_{D})=˜85 (c=1.55, MeOH). ¹H-NMR (400 MHz, CDCl₃) δ 0.80-0.94 (m, 10H), 0.97 (s, 3H), 1.20-1.69 (m, 10H), 1.74-1.83 (m, 1H), 2.00-2.22 (m, 2H), 2.33-2.45 (m, 1H), 3.83-3.89 (m, 1H), 4.09-4.27 (m, 3H), 6.02 (br d, J˜10, 1H), 7.16 (br d, J˜8, 2H), 7.27-7.37 (m, 3H). ¹³C-NMR (100 MHz, CDCl₃) δ 13.73, 13.93, 18.73, 20.00, 22.31, 25.75, 28.03, 28.26, 28.46, 31.36, 38.12, 44.99, 48.00, 49.37, 53.34, 53.62, 54.41, 127.56, 127.68,129.06, 139.71, 155.78, 158.83.

### Compounds 98 and 99

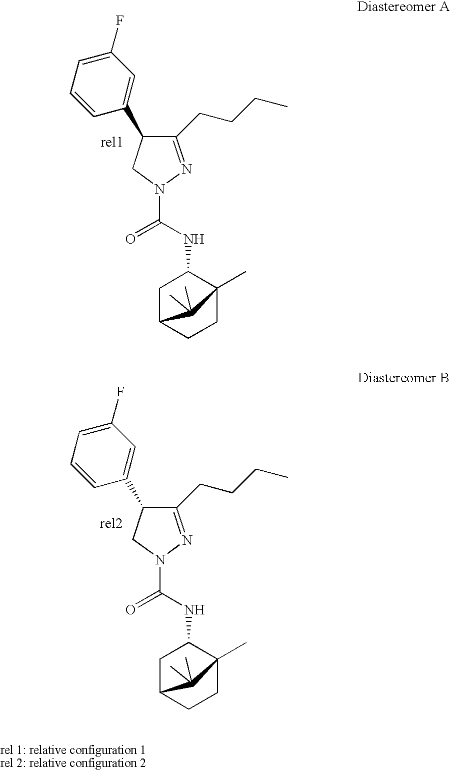

### N-Endo-[(1R,2S,4R)-1,7,7-trimethylbicyclo[2.2.1]hept-2-yl]-3-(n-butyl)-4-(3-fluorophenyl)-4,5-dihydro-(1H)-pyrazole-1-carboxamide (compound 98, diastereomer A) and N-Endo-[(1R,2S,4R)-1,7,7-trimethylbicyclo[2.2.1]hept-2-yl]-3-(n-butyl)-4-(3-fluorophenyl)-4,5-dihydro-(1H)-pyrazole-1-carboxamide (compound 99, diastereomer B)

Column chromatographic separation (gradient: petroleum ether to petroleumether/ethylacetate=4/1 (v/v)) of N-endo-[(1R,2S,4R)-1,7,7-trimethylbicyclo[2.2.1]hept-2-yl]-3-(n-butyl)-4-(3-fluorophenyl)-4,5-dihydro-(1H)-pyrazole-1-carboxamide gave compounds 98 and 99, respectively. Compound 98: Optical rotation ([α]_{D})=−116 (c=1.16, MeOH). ¹H-NMR (400 MHz, CDCl₃) δ 0.84-0.95 (m, 10H), 0.97 (s, 3H), 1.21-1.69 (m, 8H), 1.73-1.84 (m, 1H), 2.02-2.11 (m, 1H), 2.16-2.26 (m, 1H), 2.35-2.45 (m, 1H), 3.86 (dd, J=11 and 7, 1H), 4.09-4.23 (m, 3H), 6.01 (br d, J˜9, 1H), 6.88 (br d, J˜8, 1H), 6.94-7.02 (m, 2H), 7.27-7.34 (m, 1H).

Compound 99: Optical rotation ([α]_{D})=+127 (c=1.0, MeOH). ¹H-NMR (400 MHz, CDCl₃) δ 0.84-0.95 (m, 10H), 0.97 (s, 3H), 1.21-1.69 (m, 8H), 1.73-1.84 (m, 1H), 2.02-2.11 (m, 1H), 2.16-2.26 (m, 1H), 2.35-2.45 (m, 1H), 3.86 (dd, J=11 and 7, 1H), 4.09-4.23 (m, 3H), 6.01 (br d, J˜9, 1H), 6.88 (br d, J˜8, 1H), 6.94-7.02 (m, 2H), 7.27-7.34 (m, 1H).

### Compounds 100 and 101

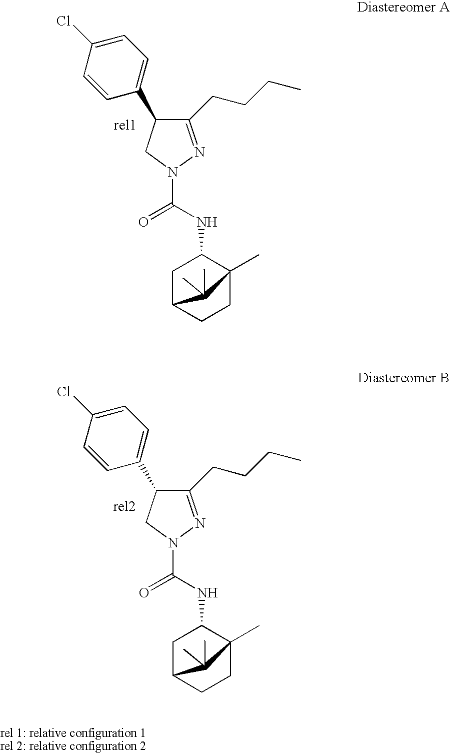

### N-Endo-[(1R,2S,4R)-1,7,7-trimethylbicyclo[2.2.1]hept-2-yl]-3-(n-butyl)-4-(4-chlorophenyl)-4,5-dihydro-(1H)-pyrazole-1-carboxamide (compound 100, diastereomer A) and N-Endo-[(1R,2S,4R)-1,7,7-trimethylbicyclo[2.2.1]hept-2-yl]-3-(n-butyl)-4-(4-chlorophenyl)-4,5-dihydro-(1H)-pyrazole-1-carboxamide (compound 101, diastereomer B)

Column chromatographic separation (gradient: petroleum ether to petroleumether/ethylacetate=4/1 (v/v)) of N-endo-[(1R,2S,4R)-1,7,7-trimethylbicyclo[2.2.1]hept-2-yl]-3-(n-butyl)-4-(4-chlorophenyl)-4,5-dihydro-(1H)-pyrazole-1-carboxamide gave compounds 31 and 32, respectively.

Compound 100: Optical rotation ([α]_{D})=−120 (c=1.0, MeOH). ¹H-NMR (400 MHz, CDCl₃) δ 0.82-0.94 (m, 10H), 0.97 (s, 3H), 1.20-1.69 (m, 8H), 1.73-1.84 (m, 1H), 2.00-2.09 (m, 1H), 2.13-2.23 (m, 1H), 2.34-2.44 (m, 1H), 3.83 (dd, J=10.7 and 6.3, 1H), 4.07-4.23 (m, 3H), 6.01 (brd, J˜9, 1H), 7.11 (br d, J=8.4, 2H), 7.32 (br d, J=8.4, 2H).

Compound 101: Optical rotation ([α]_{D})=+169 (c=1.1, MeOH). ¹H-NMR (400 MHz, CDCl₃) δ 0.82-0.92 (m, 10H), 0.97 (s, 3H), 1.20-1.69 (m, 8H), 1.73-1.84 (m, 1H), 2.00-2.09 (m, 1H), 2.13-2.23 (m, 1H), 2.34-2.44 (m, 1H), 3.83 (dd, J=10.7 and 6.3, 1H), 4.07-4.23 (m, 3H), 6.01 (brd, J˜9, 1H), 7.11 (brd, J=8.4, 2H), 7.32 (br d, J=8.4, 2H).

### Compound 102

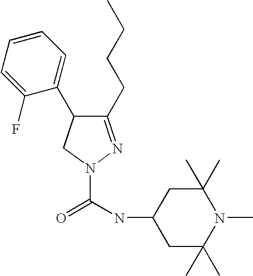

### N-(1,2,2,6,6-pentamethylpiperidin-4-yl)-3-(n-butyl)-4-(2-fluorophenyl)-4,5-dihydro-(1H)-pyrazole-1-carboxamide

To a magnetically stirred solution of 3-(n-butyl)-4-(2-fluorophenyl4,5-dihydro-(1H)-pyrazole-1-carbonyl chloride (Intermediate VIII-1) (1.26 g, 4.5 mmol) in dichloromethane (25 ml) was slowly added 1,2,2,6,6-pentamethylpiperidine (1.97 g, 11.6 mmol dissolved in 10 ml dichloromethane) and the resulting mixture was stirred for 16 hours at room temperature. The mixture was poured into water. The organic layer was separated and collected, dried over Na₂SO₄, filtered and concentrated in vacuo and subsequently purified by column chromatography (eluant: dichloromethane/methanol/25% aqueous ammonia=87.5/12/0/5 (v/v)) to give pure N-(1,2,2,6,6-pentamethylpiperidin-4-yl)-3-(n-butyl)-4-(2-fluorophenyl)-4,5-dihydro-(1H)-pyrazole-1-carboxamide (1.35 g, 73% yield)

LC/MS method C: Retention time: 1.27 minutes: Found molecular mass=417.

### Compound 103

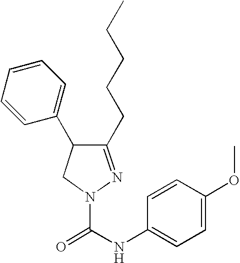

### N-(4-methoxyphenyl)-3-(n-pentyl)-4-phenyl-4,5-dihydro-(1H)-pyrazole-1-carboxamide

Compound 34 was prepared analogously to the procedure described for 34 U from 3-(n-pentyl)-4-phenyl-4,5-dihydro-(1H)-pyrazole-1-carbonyl chloride (Intermediate VIII-2) in dichloromethane in the presence of 1.2 mol equivalent DIPEA and 1.0 mol equivalent para-methoxyaniline. The mixture was reacted for 18 hours at 30° C. to give N-(4-methoxyphenyl)-3-(n-pentyl)-4-phenyl-4,5-dihydro-(1H)-pyrazole-1-carboxamide.

LC/MS method C: Retention time: 3.28 minutes: Found molecular mass=366.

Analogously were prepared compounds 104-123:

### Compound 104

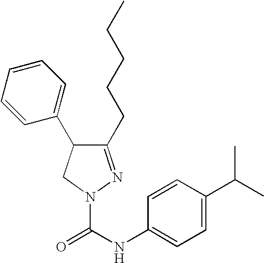

### N-(4-methoxyphenyl)-3-(n-pentyl)-4-phenyl-4,5-dihydro-(1H)-pyrazole-1-carboxamide

LC/MS method C: Retention time: 3.74 min; Found molecular mass=378.

### Compound 105

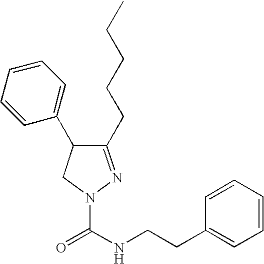

### N-(phenethyl)-3-(n-pentyl)-4-phenyl-4,5-dihydro-(1H)-pyrazole-1-carboxamide

LC/MS method C: Retention time: 3.34 min; Found molecular mass=364.

### Compound 106

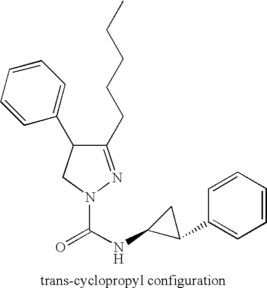

### N-(2-phenyl-trans-cyclopropyl)-3-(n-pentyl)-4phenyl-4,5-dihydro-(1H)-pyrazole-1-carboxamide

LC/MS method C: Retention time: 3.40 min; Found molecular mass=376.

### Compound 107

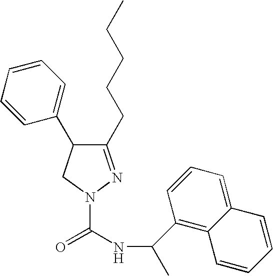

### N-(1-naphthalen-1-yl-ethyl)-3-(n-pentyl)-4-phenyl-4,5-dihydro-(1H)-pyrazole-1-carboxamide

LC/MS method C: Retention time: 3.61 min; Found molecular mass=414.

### Compound 108

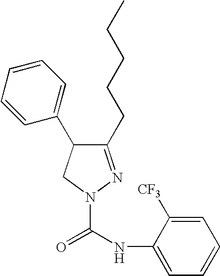

### N-[2-(trifluoromethyl)phenyl)-3-(n-pentyl)-4-phenyl-4,5-dihydro-(1H)-pyrazole-1-carboxamide

LC/MS method C: Retention time: 3.81 min; Found molecular mass=404.

### Compound 109

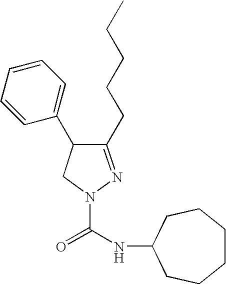

### N-cycloheptyl-3-(n-pentyl)-4-phenyl-4,5-dihydro-(1H)-pyrazole-1-carboxamide

LC/MS method C: Retention time: 3.74 min; Found molecular mass=356.

### Compound 110

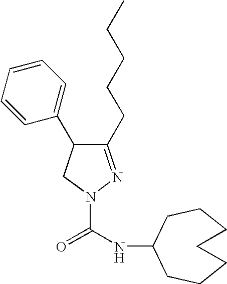

### N-cyclooctyl-3-(n-pentyl)-4-phenyl-4,5-dihydro-(1H)-pyrazole-1-carboxamide

LC/MS method C: Retention time: 3.81 min; Found molecular mass=370.

### Compound 111

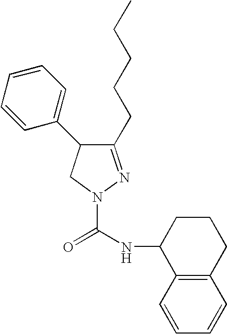

### N-(1,2,3,4tetrahydronaphthalen-1-yl)-3-(n-pentyl)-4-phenyl-4,5-dihydro-(1H)-pyrazole-1-carboxamide

LC/MS method C: Retention time: 3.61 min; Found molecular mass=390.

### Compound 112

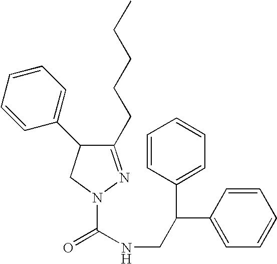

### N-[2,2-(diphenyl)ethyl]-3-(n-pentyl)-4-phenyl-4,5-dihydro-(1H)-pyrazole-1-carboxamide

LC/MS method C: Retention time: 3.59 min; Found molecular mass=440.

### Compound 113

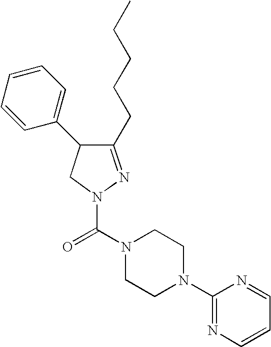

### (3-Pentyl-4-phenyl-4,5-dihydropyrazol-1-yl)-[4-(2-pyrimidinyl)piperazin-1-yl]methanone

LC/MS method C: Retention time: 3.13 min; Found molecular mass=407.

### Compound 114

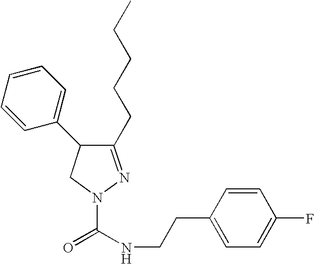

### N-[2-(4-fluorophenyl)ethyl]-3-(n-pentyl)-4-phenyl-4,5-dihydro-(1H)-pyrazole-1-carboxamide

LC/MS method C: Retention time: 3.21 min; Found molecular mass=382.

### Compound 115

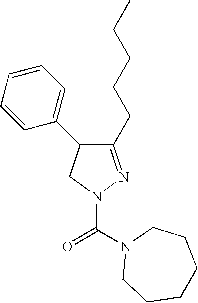

### (3-Pentyl-4phenyl-4,5-dihydropyrazol-1-yl)-[azepan-1-yl]methanone

LC/MS method C: Retention time: 3.59 min; Found molecular mass=342.

### Compound 116

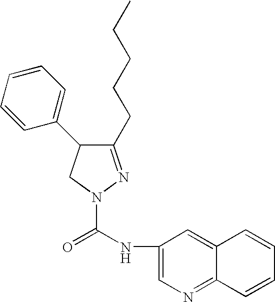

### N-(quinolin-3-yl)-3-(n-pentyl)-4-phenyl-4,5-dihydro-(1H)-pyrazole-1-carboxamide

LC/MS method C: Retention time: 3.08 min; Found molecular mass=387.

### Compound 117

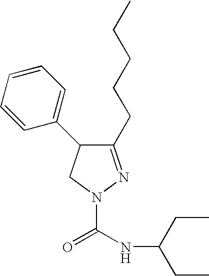

### N-[1-(ethyl)propyl]-3-(n-pentyl)-4phenyl-4,5-dihydro-(1H)-pyrazole-1-carboxamide

LC/MS method C: Retention time: 3.30 min; Found molecular mass=330.

### Compound 118

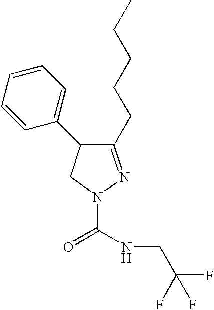

### N-(2,2,2-trifluoroethyl)-3-(n-pentyl)-4-phenyl-4,5-dihydro-(1H)-pyrazole-1-carboxamide

LC/MS method C: Retention time: 2.87 min; Found molecular mass=342.

### Compound 119

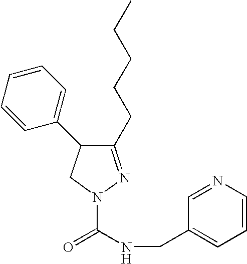

### N-(pyridin-3-ylmethyl)-3-(n-pentyl)-4phenyl-4,5-dihydro-(1H)-pyrazole-1-carboxamide

LC/MS method C: Retention time: 2.41 min; Found molecular mass=351.

### Compound 120

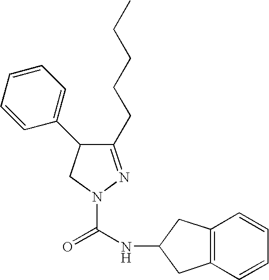

### N-(2-indanyl)-3-(n-pentyl)-4-phenyl-4,5-dihydro-(1H)-pyrazole-1-carboxamide

LC/MS method C: Retention time: 3.27 min; Found molecular mass=376.

### Compound 121

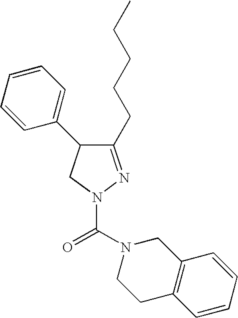

### (3-Pentyl-4phenyl-4,5-dihydropyrazol-1-yl)-(1,2,3,4-tetrahydroisoquinolin-2-yl)methanone

LC/MS method C: Retention time: 3.48 min; Found molecular mass=376.

### Compound 122

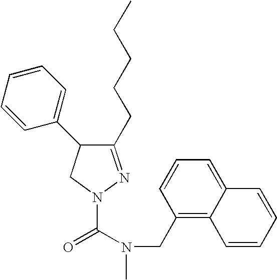

### N-(Methyl), N-(naphthalen-1-ylmethyl)-3-(n-pentyl)-4phenyl-4,5-dihydro-(1H)-pyrazole-1-carboxamide

LC/MS method C: Retention time: 3.62 min; Found molecular mass=414.

### Compound 123

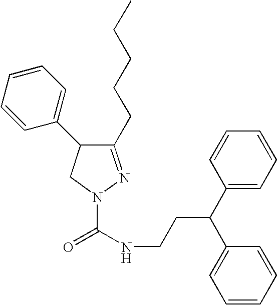

### N-(3,3-Diphenypropyl)-3-(n-pentyl)-4phenyl-4,5-dihydro-(1H)-pyrazole-1-carboxamide

LC/MS method C: Retention time: 3.59 min; Found molecular mass=454.

### Compound 124

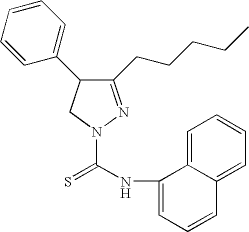

### N-(napht-1-yl)-3-(n-pentyl)-4-phenyl-4,5-dihydro-(1H)-pyrazolecarbothiamide

N-(napht-1-yl)-3-(n-pentyl)-4-phenyl-4,5-dihydro-(1H)-pyrazolecarbothiamide was obtained from 3-(n-pentyl)-4-phenyl-4,5-dihydropyrazole and an equimolar amount of 1-napthylisothiocyanate in tetrahydrofuran at 30° C. for 5 hours. LC/MS (Method C). Retention time: 3.65 min; Found molecular mass=402.

### Compound 125

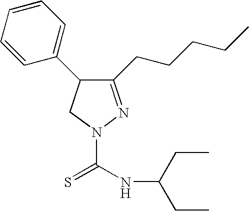

### N-[1-(ethyl)propyl]-3-(n-pentyl)-4phenyl-4,5-dihydro-(1H)-pyrazolecarbothiamide

N-(1-(ethyl)propyl)-3-(n-pentyl)-4-phenyl-4,5-dihydro-(1H)-pyrazolecarbothiamide was obtained from 3-(n-pentyl)-4-phenyl-4,5-dihydropyrazole and an equimolar amount of 1-(ethyl)propylisothiocyanate in tetrahydrofuran at 30° C. for 5 hours. LC/MS (Method C). Retention time: 3.69 min; Found molecular mass=346.

### Compound 126

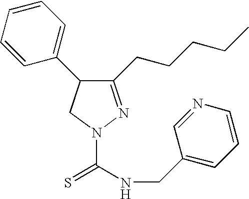

### N-[pyridin-3-ylmethyl]-3-(n-pentyl)-4-phenyl-4,5-dihydro-(1H)-pyrazolecarbothiamide

N-(pyridine-3-ylmethyl)-3-(n-pentyl4-phenyl-4, 5-dihydro-(1H)-pyrazolecarbothiamide was obtained from 3-(n-pentyl4-phenyl-4,5-dihydropyrazole and an equimolar amount of pyridin-3-ylmethylisothiocyanate in tetrahydrofuran at 30° C. for 5 hours. LC/MS (Method C). Retention time: 3.83 min; Found molecular mass=367.

### Compound 127

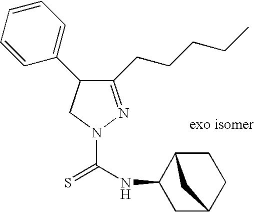

### N-[exo-bicyclo[2.2.1]hept-2-yl]-3-(n-pentyl)-4-phenyl-4,5-dihydro-(1H)-pyrazolecarbothiamide

N-[exo-bicyclo[2.2.1]hept-2-yl]-3-(n-pentyl)-4-phenyl-4,5-dihydro-(1H)-pyrazolecarbothiamide was obtained from 3-(n-pentyl)-4-phenyl-4,5-dihydropyrazole and an equimolar amount of racemic exo-bicyclo[2.2.1]hept-2-ylisothiocyanate in tetrahydrofuran at 30° C. for 5 hours. LC/MS (Method C). Retention time: 3.89 min; Found molecular mass=370.

### Compound 128

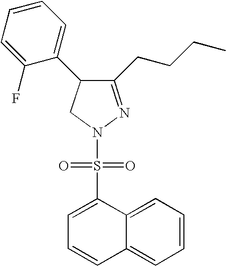

### 1-(Naphthalen-1-ylsulfonyl)-3-(n-butyl)-4-(2-fluorophenyl)-4,5-dihydro-(1H)-pyrazole

Crude 3-(n-butyl)-4-(2-fluorophenyl)-4,5-dihydropyrazole (Intermediate IV-3) (1.50 gram, 5.71 mmol maximally) was dissolved in dichloromethane (20 ml) and DIPEA (0.81 g, 1.09 ml, 6.28 mmol) and 1-naphthalenesulfonyl chloride (1.42 g, 6.28 mmol dissolved in 10 ml dichloromethane ) were successively added and the resulting magnetically stirred solution was reacted at room temperature for 16 hours. The resulting mixture was poured into water. The organic layer was separated and collected, dried over Na₂SO₄, filtered and concentrated, followed by flash chromatographic purification (dichloromethane) to give 1-(naphthalen-1-ylsulfonyl)-3-(n-butyl)-4-(2-fluorophenyl4,5-dihydro-(1H)-pyrazole (2.07 g, 88% yield). R_{f}=0.4 (dichloromethane).

LC/MS (Method D). Retention time: 2.04 min; Found molecular mass=411.

Analogously was Prepared:

### Compound 129

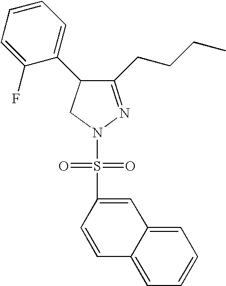

### 1-(Naphthalen-2-ylsulfonyl)-3-(n-butyl)-4-(2-fluorophenyl)-4,5-dihydro-(1H)-pyrazole

LC/MS (Method D). Retention time: 2.00 min; Found molecular mass=411.

### Compound 130

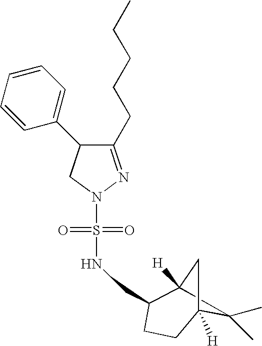

### N-[(1R,2S,5R)-rel-6,6-dimethylbicyclo[3.1.1]heptan-2-methyl]-3-(n-pentyl)-4-phenyl-4,5-dihydro-(1H)-pyrazole-1-sulfonamide

Part A: To a magnetically stirred solution of (−)cis-myrtanylamine (2.0 g, 13 mmol) (CAS 38235-68-6)) in dichloromethane (25 ml) was added triethylamine (4 ml) and chlorosulfonic acid (0.865 ml, 13 mmol, dissolved in dichloromethane (5 ml)) at 0° C. The resulting solution was allowed to attain room temperature and reacted for 16 hours.

The reaction mixture was poured in excess 1M hydrochloric acid. The precipitated crude [(1R,2S,5R)-rel-6,6-dimethylbicyclo[3.1.1]heptan-2-methyl]sulfamic acid (3.41 gram) was collected by filtration.

Part B: To a magnetically stirred solution of crude [(1R,2S,5R)-rel-6,6-dimethylbicyclo[3.1.1]heptan-2-methyl]sulfamic acid (3.41 9) in dichloromethane (25 ml) was slowly added POCl₃ (2.78 ml POCl₃ dissolved in dichloromethane (25 ml)). The resulting mixture was heated at reflux temperature for 16 hours. Subsequent concentration in vacuo gave crude [(1R,2S, 5R)-rel-6,6-dimethylbicyclo[3.1.1]heptan-2-methyl]sulfamic acid chloride (5.31 g). ¹H-NMR (300 MHz, CDCl₃) δ 0.95 (d, J=10, 1H), 1.04 (s, 3H), 1.23 (s, 3H), 1.43-1.60 (m, 1H), 1.82-2.09 (m, 5H), 2.25-2.46 (m, 2H), 3.25-3.40 (m, 2H), 5.66 (br s, 1H).

Part C: 3-(n-Pentyl)-4-phenyl-4,5-dihydropyrazole (3.4 gram, 15.7 mmol) was dissolved in toluene (25 ml) and treated with crude [(1R,2S,5R)-rel-6,6-dimethylbicyclo[3.1.1]heptan-2-methyl]sulfamic acid chloride (5.31 g, 15.7 mmol maximally) and triethylamine (2.2 ml, 15.7 mmol) and the resulting solution was magnetically stirred at room temperature for 96 hours. The solution was concentrated to give a crude oil (7.7 gram). Column chromatographic purification (heptane/ethylacetate=1:1 (v/v), followed by another column chromatographic separation using as eluant heptane/ethylacetate=6:1 (v/v ) gave N-[(1R,2S,5R)-rel-6,6-dimethylbicyclo[3.1.1]heptan-2-methyl]-3-(n-pentyl)-4-phenyl-4,5-dihydro-(1H)-pyrazole-1-sulfonamide (675 mg) as an oil. R_{f}=0.3 (heptane/ethylacetate=6:1 (v/v)). ¹H-NMR (400 MHz, CDCl₃) δ 0.83 (t, J=7, 3H), 0.93 (d, J=10, 1H), 1.01 (s, 3H), 1.20-1.29 (m, 7H), 1.41-1.60 (m, 3H), 1.85-2.43 (m, 9H), 3.22-3.28 (m, 2H), 3.64-3.71 (m, 1H), 4.02-4.09 (m, 1H), 4.12-4.19 (m, 1H), 4.66 (br t, J=7, 1H), 7.19-7.23 (m, 2H), 7.28-7.38 (m, 3H).

### Example 5

### Formulations used in Animal Studies

For oral (p.o.) administration: to the desired quantity (0.5-5 mg) of the solid compound 1 in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1N). Remaining particles in the suspension were further suspended by using an ultrasonic bath.

For intraperitoneal (i.p.) administration: to the desired quantity (0.5-15 mg) of the solid compound 1 in a glass tube, some glass beads were added and the solid was milled by vortexing for 2 minutes. After addition of 1 ml of a solution of 1% methylcellulose and 5% mannitol in water, the compound was suspended by vortexing for 10 minutes. Finally the pH was adjusted to 7.

### Example 6

### Pharmacological Methods

In Vitro Affinity for Cannabinoid-CB₁ Receptors

The affinity of the compounds of the invention for cannabinoid CB₁ receptors can be determined using membrane preparations of Chinese hamster ovary (CHO) cells in which the human cannabinoid CB₁ receptor is stably transfected in conjunction with [³H]CP-55,940 as radioligand. After incubation of a freshly prepared cell membrane preparation with the [³H]-ligand, with or without addition of compounds of the invention, separation of bound and free ligand is performed by filtration over glassfiber filters. Radioactivity on the filter is measured by liquid scintillation counting.

In Vitro Affinity for Cannabinoid-CB₂ Receptors

The affinity of the compounds of the invention for cannabinoid CB₂ receptors can be determined using membrane preparations of Chinese hamster ovary (CHO) cells in which the human cannabinoid CB₂ receptor is stably transfected in conjunction with [³H]CP-55,940 as radioligand. After incubation of a freshly prepared cell membrane preparation with the [³H]-ligand, with or without addition of compounds of the invention, separation of bound and free ligand is performed by filtration over glassfiber filters. Radioactivity on the filter is measured by liquid scintillation counting.

In Vitro Cannabinoid-CB₁Receptor (Ant)Agonism

In vitro CB₁ receptor antagonism/agonism can be assessed with the human CB₁ receptor cloned in Chinese hamster ovary (CHO) cells. CHO cells are grown in a Dulbecco's Modified Eagle's medium (DMEM) culture medium, supplemented with 10% heat-inactivated fetal calf serum. Medium is aspirated and replaced by DMEM, without fetal calf serum, but containing [³H]-arachidonic acid and incubated overnight in a cell culture stove (5% CO₂/95% air; 37° C.; water-saturated atmosphere). During this period [³H]-arachidonic acid is incorporated in membrane phospholipids. On the test day, medium is aspirated and cells are washed three times using 0.5 ml DMEM, containing 0.2% bovine serum albumin (BSA). CB₁ agonist stimulation leads to activation of PLA₂ followed by release of [³H]-arachidonic acid into the medium. This CB₁ agonist-induced release is concentration-dependently antagonized by CB₁ receptor antagonists, such as for example rimonabant.

In Vitro Cannabinoid-CB₂ Receptor (Ant)Agonism

Functional activity at the cannabinoid CB₂ receptor was assessed using a forskolin-stimulated cAMP accumulation assay. The ability of compounds to stimulate and inhibit adenylate cyclase activity was assessed in Chinese ovarian hamster (CHO) K₁ cells expressing human CB2 (Euroscreen, Brussel) receptor. CHO cells were grown in a CHO—S—SFM-II culture medium, supplemented with 10% heat-inactivated foetal calf serum, 2 mM glutamine, 400 μg/ml Hygromycine B and 500 μg/ml G418 at 37° C. in 93% air/5% CO₂. For incubation with test compounds, confluent cultures grown in 24 well plates were used. Each condition or substance was routinely tested in quadruplicate. Cells were loaded with 1 mCi [³H]-adenine in 0.5 ml medium per well. After 2 hours, cultures were washed with 0.5 ml PBS containing 1 mM IBMX and incubated for 20 minutes with 0.5 ml PBS containing 1 mM IBMX and 3×10⁻⁷ M forskolin with or without the test compound. Antagonistic effects of test compounds were determined as inhibition of 0.1 μM JWH-133-decreased [³H]cAMP formation. After aspiration the reaction was stopped with 1 ml trichloroacetic acid (5% w/v). The [3H]-ATP and [³H]-cAMP formed in the cellular extract were assayed as follows: a volume of 0.8 ml of the extract was passed over Dowex (50WX-4200-400 mesh) and aluminum oxide columns, eluted with water and 0.1 M imidazole (pH=7.5). Eluates were mixed with 7 ml Ultima-Flo [AP] and the β-radioactivity was counted with a liquid scintillation counter. The conversion of [³H]-ATP into [³H]-cAMP was expressed as the ratio in percentage radioactivity in the cAMP fraction as compared to the combined radioactivity in both cAMP and ATP fractions, and basal activity was subtracted to correct for spontaneous activity. Reference compounds used to assess cannabinoid CB₂ receptor mediated adenylate cyclase activity were the full cannabinoid CB₂ receptor agonists JWH-133 (Huffman, 1999^{b}) and WIN 55, 212-2 (Huffman, 1999^{a}), and the inverse agonist or antagonist SR-144528 (Rinaldi-Carmona, 1998). Compounds were studied in a concentration range of 10⁻¹⁰ M to 10⁻⁶M. pEC₅₀ and the pA₂ were calculated according to Cheng-Prusoff equation (Cheng and Prusoff, 1973). Two independent experiments were performed in triplicate.

### Example 7

### Pharmacological Testresults

Cannabinoid CB₁/CB₂ receptor affinity data, expressed as pKᵢ values (mean results of at least three independent experiments, performed according to the protocols given above) as well as CB₁ receptor agonist functional data of representative compounds of this invention are shown in the table below.

TABLE 1
CB₁ and CB₂ receptor affinities and CB₁ functional agonistic activity of
representative compounds of this invention.

| | | | Human CB₁ |
|---|---|---|---|
| | Human CB₁ | Human CB₂ | Arachidonic acid |
| | receptor binding | | release (CB₁) |
| Compound | pKi | pK₁ | pEC₅₀ |
| 1 | 8.4 | 7.9 | 5.7 |
| 7 | 7.4 | 8.0 | 6.2 |
| 11 | 7.8 | 8.1 | 5.7 |
| 12 | 7.8 | 7.3 | |
| 13 | 8.1 | 8.1 | 9.3 |
| 15 | 7.0 | 7.8 | |
| 18 | 8.2 | 7.5 | |
| 39 | 5.4 | 6.9 | |
| 40 | 5.8 | 6.8 | |
| 41 | 6.5 | 6.7 | |
| 54 | 7.6 | 7.4 | 6.6 |
| 87 | 7.1 | 7.0 | 7.8 |
| 97 | 8.3 | 8.3 | >9.0 |
| 109 | 8.2 | 7.6 | |
| 130 | 6.5 | 6.9 | |

These data illustrate the affinities of representative compounds for the CB₁ and CB₂ receptor as well as the CB₁ agonistic properties achieved by the structural modifications forming the basis of the present invention.

TABLE 2
CB₂ functional agonistic/antagonistic activity of representative
compounds of this invention.

| | Human CB₂ | Human CB₂ |
|---|---|---|
| | CB₂-mediated adenylate | CB₂-mediated adenylate |
| | cyclase activity | cyclase activity |
| Compound | pEC₅₀ | pA₂ |
| 39 | 8.1 | |
| 40 | 8.1 | |
| 97 | | 9.2 |

These data illustrate the functional cannabinoid-CB₂ agonistic or antagonistic acivity of representative compounds of the present invention.

### Example 8

### Pharmaceutical Preparations

For clinical use, compounds of formula (I) are formulated into a pharmaceutical compositions which are important and novel embodiments of the invention because of the presence of the compounds, more particularly specific compounds disclosed herein. Types of pharmaceutical compositions that may be used include but are not limited to tablets, chewable tablets, capsules (including microcapsules), solutions, parenteral solutions, ointments (creams and gels), suppositories, suspensions, and other types disclosed herein or apparent to a person skilled in the art from the specification and general knowledge in the art. The compositions are used for oral, intravenous, subcutaneous, tracheal, bronchial, intranasal, pulmonary, transdermal, buccal, rectal, parenteral or some other mode of administration. The pharmaceutical formulation contains at least one compound of formula (I) in admixture with a pharmaceutically acceptable adjuvant, diluent and/or carrier. The total amount of active ingredients suitably is in the range of from about 0.1% (w/w) to about 95% (w/w) of the formulation, suitably from 0.5% to 50% (w/w) and preferably from 1% to 25% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances which may be mentioned are magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets.

The active ingredients may be separately premixed with the other non-active ingredients, before being mixed to form a formulation. The active ingredients may also be mixed with each other, before being mixed with the non-active ingredients to form a formulation.

Soft gelatine capsules may be prepared with capsules containing a mixture of the active ingredients of the invention, vegetable oil, fat, or other suitable vehicle for soft gelatine capsules. Hard gelatine capsules may contain granules of the active ingredients. Hard gelatine capsules may also contain the active ingredients in combination with solid powdered ingredients such as lactose, saccharose, sorbitol, mannitol, potato starch, corn starch, amylopectin, cellulose derivatives or gelatine. Dosage units for rectal administration may be prepared (i) in the form of suppositories which contain the active substance mixed with a neutral fat base; (ii) in the form of a gelatine rectal capsule which contains the active substance in a mixture with a vegetable oil, paraffin oil or other suitable vehicle for gelatine rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder to be reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation to be reconstituted with a suitable solvent before use.

Also provided according to the present invention are formulations and ‘kits of parts’ comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. The use of formulations of the present invention in the manufacture of medicaments for use in the treatment of a condition in which modulation of cannabinoid CB₁ receptors is required or desired, and methods of medical treatment or comprising the administration of a therapeutically effective total amount of at least one compound of formula (I), either as such or, in the case of prodrugs, after administration, to a patient suffering from, or susceptible to, a condition in which modulation of cannabinoid CB, receptors is required or desired.

The invention has been described with reference to various specific and preferred embodiments and techniques. However, it should be understood that many variations and modifications may be made while remaining within the spirit and scope of the invention.

### REFERENCES

Albericio, F., et al., Tetrahedron Lett., 38, 4853-4856, 1997.
Akaji, K. et al., Tetrahedron Lett., 35, 3315-3318, 1994.
Barluenga et al. Chem. Eur. J., 5, (3) 883-896, 1999
Bickel, M. H., “The pharmacology and Biochemistry of N-oxides”, Pharmacological Reviews, 21(4), 325-355, 1969.
Bodanszky, M. and A. Bodanszky: The Practice of Peptide Synthesis, Springer-Verlag, New York, ISBN: 0-387-57505-7, 1994.
Bundgaard, H. (editor), “Design of Prodrugs”, Elsevier, 1985.
Cheng, Y. and Prusoff, W. H., Biochem. Pharmacol., 22, 3099-3108, 1973
De Petrocellis, L. et al,. Br. J. Pharmacol., 141, 765-774, 2004.
Di Marzo, V. et al., Nature Rev. Drug Discov., 3, 771-784, 2004.
Ettmayer, P. et al., “Lessons learned from marketed and investigational prodrugs”, J. Med. Chem., 47, 2393-2404, 2004.
Hertzog, D. L. Expert Opin. Ther. Patents, 14, 1435-1452, 2004
Huffman et al., Curr. Med. Chem., 6, 705-720, 1999
Huffman et al., Bioorg. Med. Chem., 7, 2905-2914, 1999
Järvinen, T., “Design and Pharmaceutical applications of prodrugs”, p. 733-796 in: S. C. Gad “Drug Discovery Handbook”, John Wiley & Sons, New Jersey, U.S.A., 2005.
King, F. D., (editor), page 215 in: “Medicinal Chemistry: Principles and Practice”, 1994.
Lambert, D. M. and Fowler, C. J. J. Med. Chem., 48, 5059-5087, 2005.
Lange, J. H. M. et al., J. Med. Chem., 47, 627-643, 2004.
Lange, J. H. M. et al., Bioorg. Med. Chem. Lett, 15, 4794-4798, 2005.
Lange, J. H. M. and Kruse, C. G., C. Cuff. Opin. Drug Discovery Dev, 7, 498-506, 2004
Lange, J. H. M. and Kruse, C. G. Drug Discov. Today, 10, 693-702, 2005;
Montalbetti, C. A. G. N. & V. Falque, Tetrahedron, 61, 10827-52. 2005.
Muccioli, G. G. et al., Curr. Med. Chem., 12,1361-1394, 2005
Muccioli, G. G. and Lambert, D. M., Expert Opin. Ther. Patents, 16, 1405-1423, 2006
Ogata, J., et al., J. Med. Chem. 1987, 30, 1054-1068, 1987^{a}
Ogata, J., et al., J. Med. Chem. 1987, 30, 1497-1502, 1987^{b}
Padgett, L. W. Life Sc., 77, 1767-1798, 2005.
Raitio, K. H., et al., Curr. Med. Chem., 12, 1217-1237, 2005.
Rinaldi-Carmona et al., J. Pharmacol. Exp. Ther., 284, 644-650, 1998
Smith, R. A. and Fathi, Z. IDrugs, 8, 53-66, 2005.
Stella, J., “Prodrugs as therapeutics”, Expert Opin. Ther. Patents, 14(3), 277-280, 2004.
Thakur, G. A. et al., Mini-Rev. Med. Chem., 5, 631-640, 2005.
Vandevoorde, S. and Lambert, D. M. Curr. Pharm. Des., 11, 2647-68, 2005
Van Sickle, M. D. et al., Science, 310, 329-332, 2005
Wang et al., Synth. Commun., 33 (9), 1449-1457, 2003.

1. Compounds of the general formula (I) 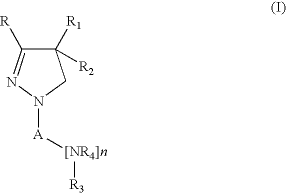
wherein
R represents a C₂₋₁₀ alkyl group, a C₄₋₁₀ alkenyl group, a C₄₋₁₀ alkynyl group, a C₂₋₁₀-heteroalkyl group, a C₅₋₈-cycloalkyl-C₁₋₅-alkyl group or a C₅₋₈-heterocycloalkyl-C₁₋₅-alkyl group wherein the heteroatom(s) are either N, O or S, which C₂₋₁₀ alkyl group, C₄₋₁₀ alkenyl group, C₄₋₁₀ alkynyl group, C₂₋₁₀-heteroalkyl group, C₅₋₈-cycloalkyl-C₁₋₅-alkyl group or C₅₋₈-heterocycloalkyl-C₁₋₅-alkyl group may be substituted with 1-5 substituents selected from methyl, ethyl, hydroxy, amino or fluoro, or R represents an aryl-C₁₋₃-alkyl group or an aryl-C₁₋₃-heteroalkyl group in which the aryl groups may be substituted with 1-5 substituents Y, which can be the same or different, selected from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethyl-sulfonyl, cyano, carbamoyl, sulfamoyl, phenyl and acetyl, or R represents a cyclopropyl group which cyclopropyl group may be substituted with 1-5 substituents selected from methyl, ethyl, fluoro or with a C₃₋₅ linear or branched alkyl group or with a benzyl or aryl group, in which the aryl or benzyl group may be substituted with 1-5 substituents Y,
R¹ represents hydrogen, hydroxy, C₁₋₃-alkoxy, acetyloxy or propionyloxy,
R₂ represents an aryl group which may be substituted with 1-5 substituents Y, wherein Y has the abovementioned meaning,
n is either 0 or 1
R₃ represents a linear C₃₋₁₀ alkyl group, a branched C₅₋₁₀ alkyl group, a cyclopropyl, cyclobutyl, cyclopentyl, cycloheptyl or cyclooctyl group, C₅₋₁₀ bicycloalkyl group, C₆₋₁₀ tricycloalkyl group or C₈₋₁₁ tetracycloalkyl group which groups may be substituted with 1-5 substituents selected from methyl, ethyl, hydroxy, amino, fluoro or R₃ represents a C₃₋₈ cycloalkyl group which C₃₋₈ cycloalkyl group is substituted with an aryl group which aryl group may be substituted with 1-5 substituents Y wherein Y has the abovementioned meaning, or R₃ represents a 2,2,2-trifluoroethyl or 2-fluoroethyl group or R₃ represents a cyclohexyl group which group is substituted with 1-5 substituents selected from methyl, ethyl, hydroxy, amino or fluoro, or R₃ represents a C₅₋₈ heterocycloalkyl group, C₆₋₁₀ bicycloheteroalkyl group, C₇₋₁₀ tricycloheteroalkyl group, which groups may be substituted with 1-5 substituents selected from methyl, ethyl, hydroxy, amino or fluoro, or R₃ represents a C₃₋₈ cycloalkyl-C₁₋₃-alkyl group, C₅₋₁₀-bicycloalkyl-C₁₋₃-alkyl group, C₆₋₁₀-tricycloalkyl-C₁₋₃-alkyl group, which groups may be substituted with 1-5 substituents selected from methyl, ethyl, hydroxy, amino or fluoro, or R₃ represents a branched or linear C₃₋₈heterocycloalkyl-C₁₋₃-alkyl group, C₅₋₁₀ bicycloheteroalkyl-C₁₋₃-alkyl group, C₆₋₁₀ tricycloheteroalkyl-C₁₋₃-alkyl group, which groups may be substituted with 1-5 substituents selected from methyl, ethyl, hydroxy, amino or fluoro, or R₃ represents an aryl group, which group may be substituted with 1-5 substituents Y, wherein Y has the abovementioned meaning, or R₃ represents a aryl-C₁₋₅alkyl group or a diaryl-C₁₋₅alkyl group, in which groups the phenyl or heteroaromatic rings may be substituted with 1-5 substituents Y, wherein Y has the abovementioned meaning, or R₃ represents a linear or branched C₄₋₈ alkenyl or C₄₋₈ alkynyl group which linear or branched C₄₋₈ alkenyl or C₄₋₈ alkynyl group may be substituted with 1-3 fluoro atoms, or, when n=1, R₃ represents a branched or linear C₂₋₁₀ heteroalkyl group, containing 1-2 heteroatoms selected from N, O or S,
R₄ represents a hydrogen atom, a C₁₋₄ alkyl group or R₃ and R₄—together with the nitrogen atom to which they are bonded—form a saturated or unsaturated, non-aromatic or partly aromatic, monocyclic, bicyclic or tricyclic heterocyclic group having 5 to 11 ring atoms, which heterocyclic group may be substituted with 1-5 substituents selected from aryl, aryl-C₁₋₃-alkyl, diarylmethyl, or Y, wherein Y has the abovementioned meaning,
A represents a carbonyl (C═O), thiocarbonyl (C═S) or sulfonyl (SO₂) group with the proviso that when A represents a thiocarbonyl (C═S),group, n has the value 1,
and stereoisomers, prodrugs and N-oxides thereof, and isotopically-labelled compounds of formula (I), as well as pharmacologically acceptable salts, hydrates, solvates, complexes and conjugates of said compounds of formula (I) and its stereoisomers, prodrugs, N-oxides, or isotopically-labelled analogs.

2. Compounds as claimed in claim 1 of the general formula (I), wherein R¹ represents a hydrogen atom, and the other symbols have the meanings as given in claim 1.

3. Compounds as claimed in claim 2 of the general formula (I) wherein A represents a carbonyl group, and the other symbols have the meanings as given in claim 2.

4. Compounds as claimed in claim 3 of the general formula (I) wherein R₂ represents a phenyl, thienyl or pyridyl group, which phenyl, pyridyl or thienyl group may be substituted with 1, 2 or 3 substituents Y, and the other symbols have the meanings as given in claim 3.

5. Compounds as claimed in claim 4 of the general formula (I) wherein n is 1, and the other symbols have the same meanings as given in claim 4

6. Compounds as claimed in claim 5 of the general formula (I) wherein R₄ represents a hydrogen atom, and the other symbols have the same meanings as given in claim 5.

7. Compounds as claimed in claim 6 of the general formula (I) wherein R represents a C₃₋₈ branched or linear alkyl group, which C₃₋₈ branched or linear alkyl group may be substituted with 1-3 fluoro atoms, and the other symbols have the same meanings as given in claim 6.

8. The compound according to claim 1 which is:
N-[(1R,2S,5R)-rel-6,6-dimethylbicyclo[3.1.1]heptan-2-methyl]-3-(n-pentyl)-4-phenyl-4,5-dihydro-(1H)-pyrazole-1-carboxamide
N-(1-Adamantyl)-3-(n-pentyl)-4-phenyl-4,5-dihydro-(1H)-pyrazole-1-carboxamide
N-(Exo-bicyclo[2.2.1]hept-2-yl)-3-(n-pentyl)-4-phenyl-4,5-dihydro-(1H)-pyrazole-1-carboxamide
N-Phenyl-3-(n-pentyl)-4-phenyl-4,5-dihydro-(1H)-pyrazole-1-carboxamide
N-[(1R,2S, 5R)-rel-6,6-dimethylbicyclo[3.1.1]heptan-2-methyl]-3-(benzyl)-4-phenyl-4,5-dihydro-(1H)-pyrazole-1-carboxamide
N-(1-Adamantyl)-3-(benzyl)-4-phenyl-4,5-dihydro-(1H)-pyrazole-1-carboxamide
N-[(1R,2S,5R)-rel-6,6-dimethylbicyclo[3.1.1]heptan-2-methyl]-3-(n-butyl)-4-phenyl-4,5-dihydro-(1H)-pyrazole-1-carboxamide
N-[(1R,2S,5R)-rel-6,6-dimethylbicyclo[3.1.1]heptan-2-methyl]-3-[3-(1-piperidinyl)propyl]-4-phenyl-4,5-dihydro-(1H)-pyrazole-1-carboxamide
N-[(1R,2S,5R)-rel-6,6-dimethylbicyclo[3.1.1]heptan-2-methyl]-3-(n-propyl)-4-phenyl-4,5-dihydro-(1H)-pyrazole-1-carboxamide
N-(Benzyl)-3-(n-pentyl)-4-phenyl-4,5-dihydro-(1H)-pyrazole-1-carboxamide
N-(1-Adamantyl)methyl-3-(n-pentyl)-4-phenyl-4,5-dihydro-(1H)-pyrazole-1-carboxamide
N-(Cyclohexylmethyl)-3-(n-pentyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(n-pentyl)-4-phenyl-4,5-dihydro-(1H)-pyrazole-1-carboxamide
N-[endo-(1S)-1,3,3-trimethylbicyclo[2.2.1]hept-2-yl]-3-(n-pentyl)-4-phenyl-4,5-dihydro-(1H)-pyrazole-1-carboxamide
N-[(1R,2S,5R)-rel-6,6-dimethylbicyclo[3.1.1]heptan-2-methyl]-3-(n-propyl)-4-(2-pyridyl)-4,5-dihydro-(1H)-pyrazole-1-carboxamide
N-(1-Phenyl-ethyl)-3-(n-pentyl)-4-phenyl-4,5-dihydro-(1H)-pyrazole-1-carboxamide
N-(2-Adamantyl)-3-(n-pentyl)-4-phenyl-4,5-dihydro-(1H)-pyrazole-1-carboxamide
N-(1-Naphtyl)-3-(n-pentyl)-4-phenyl-4,5-dihydro-(1H)-pyrazole-1-carboxamide
N-(1-Methyl-1-phenyl-ethyl)-3-(n-pentyl)4-phenyl-4,5-dihydro-(1H)-pyrazole-1-carboxamide
N-(2,2-Diphenylpropyl)-3-(n-pentyl)-4-phenyl-4,5-dihydro-(1H)-pyrazole-1-carboxamide
N-((3-Trifluoromethyi)benzyl)-3-(n-pentyl)-4-phenyl-4,5-dihydro-(1H)-pyrazole-1-carboxamide
N-(2,2-Dimethylpropyl)-3-(n-pentyl)-4-phenyl-4, 5-dihydro-(1H)-pyrazole-1-carboxamide
N-(Naphthalen-1-yl-methyl)-3-(n-pentyl4-phenyl-4,5-dihydro-(1H)-pyrazole-1-carboxamide
N-[(3-Dimethylamino)-2,2-dimethylpropyl]-3-(n-pentyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-phenyl-4,5-dihydro-(1H)-pyrazole-1-carboxamide
N-(2-(4-fluorophenyl)-1,1-dimethyl-ethyl)-3-(n-butyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(4,4,4-trifluoro-n-butyl)-4-phenyl-4,5-dihydro-(1H)-pyrazole-1-carboxamide
N-(2-(4-fluorophenyl)-1,1-dimethyl-ethyl)-3-(4,4,4-trifluoro-n-butyl)-4-phenyl-4,5-dihydro-(1H)-pyrazole-1-carboxamide
N-(2-(4-Fluorophenyl)-1,1-dimethyl-ethyl)-3-(n-pentyl)-4-phenyl-4,5-dihydro-(1H)-pyrazole-1-carboxamide
N-[Endo-(1R,2S,4R)-1,7,7-Trimethylbicyclo[2.2.1]hept-2-yl]-3-(1,1-dimethyl-n-butyl)-4-phenyl-4,5-dihydro-(1H)-pyrazole-1-carboxamide
N-[Endo-(1R,2S,4R)-1,7,7-Trimethylbicyclo[2.2.1]hept-2-yl]-3-(3,3,3-trifluoropropyl)-4-phenyl-4,5-dihydro-(1H)-pyrazole-1-carboxamide
N-[Endo-(1R,2S,4R)-1,7,7-Trimethylbicyclo[2.2.1]hept-2-yl]-3-(1,1-dimethylpropyl)-4-phenyl-4,5-dihydro-(1H)-pyrazole-1-carboxamide
N-(2-(4-Fluorophenyl)-1,1-dimethyl-ethyl)-3-(1,1-dimethylpropyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(1,1-dimethyl-3,3,3-trifluoropropyl)-4-phenyl-4,5-dihydro-(1H)-pyrazole-1-carboxamide
N-[endo-(1R)-1,3,3-trimethylbicyclo[2.2.1]hept-2-yl]-3-(n-butyl)-4-phenyl-4,5-dihydro-(1H)-pyrazole-1-carboxamide
N-(1-Methyl-1-phenyl-ethyl)-3-(n-butyl)-4-phenyl-4,5-dihydro-(1H)-pyrazole-1-carboxamide
N-(2-Adamantyl)-3-(n-butyl)-4-phenyl-4,5-dihydro-(1H)-pyrazole-1-carboxamide
N-[Exo-(1R,2R,4R)-1,7,7-trimethylbicyclo[2.2.1]hept-2-yl]-3-(n-butyl)-4-phenyl-4,5-dihydro-(1H)-pyrazole-1-carboxamide
N-(2-phenyl-1,1-dimethyl-ethyl)-3-(n-butyl)-4-(3-fluorophenyl)-4,5-dihydro-(1H)-pyrazole-1-carboxamide
N-(2-phenyl-1,1-dimethyl-ethyl)-3-(n-butyl)-4-(2-fluorophenyl)-4,5-dihydro-(1H)-pyrazole-1-carboxamide
N-Phenyl-3-(4-chlorobenzyl)-4-(4-chlorophenyl)-4,5-dihydro-(1H)-pyrazole-1-carboxamide
N-(4-Methoxyphenyl)-3-(4-chlorobenzyl)-4-(4-chlorophenyl)-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-(2methoxyphenyl)-4,5-dihydro-(1H)-pyrazole-1-carboxamide
N-(1-Methyl-1-phenyl-ethyl)-3-(n-butyl)-4-(2-methoxyphenyl)-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-(2-fluorophenyl)-4,5-dihydro-(1H)pyrazole-1-carboxamide
N-[Endo-(1R,2S,4R)-1, 7,7-trimethylbicyclo[2.2. 1 ]hept-2-yl]-3-(n-butyl)-4-(pyrid-3-yl)-4,5-dihydro-(1H )-pyrazole-1-carboxamide
N-[(1R,2R,3R,5S)-2,7,7-trimethylbicyclo[3. 1.1 ]hept-3-yl]-3-(n-butyl)-4-(3-fluorophenyl)-4,5-dihydro-(1H)pyrazole-1-carboxamide
N-[endo-(1R)-1,3,3-trimethylbicyclo[2.2.1]hept-2-yl]-3-(n-butyl)-4-(2-fluorophenyl)-4,5-dihydro-(1Hypyrazole-1-carboxamide
N-[2-(trifluoromethyl)benzyl]-3-(n-butyl)-4-(3-fluorophenyl)-4,5-dihydro-(1H)-pyrazole-1-carboxamide
N-[Exo-(1R,2R,4R)-1,7,7-trimethylbicyclo[2.2.1]hept-2-yl]-3-(n-butyl)-4-(2-fluorophenyl)-4,5-dihydro-(1H )-pyrazole-1-carboxamide
N-(1-Methyl-1-phenyl-ethyl)-3-(n-butyl)-4-(3-fluorophenyl)-4,5-dihydro-(1H)-pyrazole-1-carboxamide
N-(1-Methyl-1-phenyl-ethyl)-3-(n-butyl)-4-(4-chlorophenyl)-4,5-dihydro-(1H)-pyrazole-1-carboxamide
N-[2-(trifluoromethyl)benzyl]-3-(n-butyl)-4-(4-chlorophenyl)-4,5-dihydro-(1H)-pyrazole-1-carboxamide
N-[Endo-(1R,2S,4R)-1,7,7-trimethylbicyclo[2.2.1]hept-2-yl]-3-(cyclopropylmethyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-(4-fluorophenyl)-4,5-dihydro-(1H)-pyrazole-1-carboxamide
N-(1-Methyl-1-phenyl-ethyl)-3-(n-butyl)-4-(4-fluorophenyl)-4,5-dihydro-(1H)-pyrazole-1-carboxamide
N-(Adamant-2-yl)-3-(n-butyl)-4-(4-fluorophenyl)-4,5-dihydro-(1H)-pyrazole-1-carboxamide
N-(1-Methyl-1-(4-fluorophenyl)-ethyl)-3-(n-butyl)-4-(4-fluorophenyl)-4,5-dihydro-(1H)-pyrazole-1-carboxamide
N-(1-Methyl-1-(4-fluorophenyl)-ethyl)-3-(n-butyl)-4-phenyl-4,5-dihydro-(1H)-pyrazole-1-carboxamide
N-(1-Methyl-1-phenyl-ethyl)-3-(n-pentyl)-4-(2-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(n-pentyl)-4-(2-fluorophenyl)-4, 5-dihydro-(1H)-pyrazole-1-carboxamide
N-(1-Methyl-1-(4-fluorophenyl)-ethyl)-3-(n-pentyl)-4-(2-fluorophenyl)-4,5-dihydro-(1H)-pyrazole-1-carboxamide
N-(1-Methyl-1-(4-fluorophenyl)-ethyl)-3-(n-pentyl)-4-(3-fluorophenyl)-4,5-dihydro-(1H)-pyrazole-1-carboxamide
N-(adamant-2-yl)-3-(n-pentyl)-4-(2-fluorophenyl)-4,5-dihydro-(1H)-pyrazole-1-carboxamide
N-(adamant-2-yl)-3-(n-pentyl)-4-(3-fluorophenyl)-4,5-dihydro-(1H)-pyrazole-1-carboxamide
N-(1-Methyl-1-phenyl-ethyl)-3-(n-butyl)-4-(benzo[b]thiophen-3-yl)-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-(benzo[b]thiophen-3-yl4, 5-dihydro-(1H)-pyrazole-1-carboxamide
N-(1-Methyl-1-phenyl-ethyl)-3-(n-butyl)-4-(thiophen-3-yl)-4,5-dihydro-(1H)-pyrazole-1-carboxamide
N-(1-Methyl-1-phenyl-ethyl)-3-(but-3-ynyl)-4-(2-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(but-3-ynyl)-4-(2-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(1-phenylcyclopropyl)-4-phenyl-4,5-dihydro-(1H)-pyrazole-1-carboxamide
N-(1-Methyl-1-phenyl-ethyl)-3-(1-phenylcyclopropyl)-4-phenyl-4,5-dihydro-(1H)-pyrazole-1-carboxamide
N-[Endo-(1R,2S,4R)-1,7,7-trimethylbicyclo[2.2.1]hept-2-yl]-3-(2,2,3,3-tetramethylcyclopropyl)-4-phenyl-4,5-dihydro-(1H)-pyrazole-1-carboxamide
N-(1-Methyl-1-phenyl-ethyl)-3-(2,2,3,3-tetramethylcyclopropyl)-4-phenyl-4,5-dihydro-(1H)-pyrazole-1-carboxamide
N-[(1R,2R,3R,5S)-2,7,7-trimethylbicyclo[3.1.1]hept-3-yl]-3-(n-butyl)-4-(4-chlorophenyl)-4,5-dihydro-(1H )-pyrazole-1-carboxamide
N-(1-Methyl-1-phenyl-ethyl)-3-(n-pentyl)-4-(3-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(n-pentyl)-4-(3-fluorophenyl)-4,5-dihydro-(1H)-pyrazole-1-carboxamide
N-(1-Methyl-1-phenyl-ethyl)-3-(n-pentyl)-4-(4-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(n-pentyl)-4-(4-fluorophenyl)-4,5-dihydro-(1H)-pyrazole-1-carboxamide
N-[(1S,2S,3S,5R)-2,7,7-trimethylbicyclo[3.1.1]hept-3-yl]-3-(n-butyl)-4-(3-fluorophenyl)-4,5-dihydro-(1Hypyrazole-1-carboxamide
N-(1-Methyl-1-(4-fluorophenyl)-ethyl)-3-(n-butyl)-4-(3-fluorophenyl)-4,5-dihydro-(1H)-pyrazole-1-carboxamide
N-(1-Methyl-1-(4-fluorophenyl)-ethyl)-3-(n-butyl)-4-(2-fluorophenyl)-4,5-dihydro-(1H)-pyrazole-1-carboxamide
N-[(1S,2S,3S,5R)-2,7,7-trimethylbicyclo[3.1.1]hept-3-yl]-3-(n-butyl)-4-(2-fluorophenyl)-4,5-dihydro-(1H)-pyrazole-1-carboxamide
N-[Endo-(1R,2S,4R)-1,7,7-trimethylbicyclo[2.2.1]hept-2-yl]-3-(n-butyl)-4-(thien-3-yl)-4,5-dihydro-(1H)-pyrazole-1-carboxamide
N-[Endo-(1R,2S,4R)-1,7,7-trimethylbicyclo[2.2.1]hept-2-yl]-3-(3,3,3-trifluoro-1-methoxymethyl-propyl)-4-phenyl-4,5-dihydro-(1H )-pyrazole-1-carboxamide
N-[Endo-(1R,2S,4R)-1,7,7-trimethylbicyclo[2.2.1]hept-2-yl]-3-(n-butyl)-4-hydroxy-4-phenyl-4,5-dihydro-(1H)-pyrazole-1-carboxamide
1-(1-Naphtoyl)-3-(n-pentyl)-4-phenyl-4,5-dihydro-(1H)-pyrazole
[3-(n-pentyl)-4-phenyl-4,5-dihydro-(1H)-pyrazol-1-yl]-[1-(4-chlorophenyl)cyclopentyl]methanone
(3-(n-pentyl)-4-phenyl-4,5-dihydro-(1H )-pyrazol-1-yl]-(napht-2-yl)methanone
(3-(n-pentyl)-4-phenyl-4,5-dihydro-(1H)-pyrazol-1-yl]-(diphenylmethyl)methanone
[3-(n-pentyl)-4-phenyl-4,5-dihydro-(1H)-pyrazol-1-yl]-(3-chlorobenzothien-2-yl]methanone
[3-(n-pentyl)-4-phenyl-4,5-dihydro-(1H)-pyrazol-1-yl]-(benzofuran-2-yl]methanone
[3-(n-pentyl)-4-phenyl-4,5-dihydro-(1H)-pyrazol-1-yl]-[2,4,4-(trimethyl)pentyl]methanone
[3-(n-pentyl)-4-phenyl-4,5-dihydro-(1H)-pyrazol-1-yl]-[3-(trifluoromethyl)phenyl]methanone
(Cis-3,4,5-trimethylpiperazin-1-yl)[3-(n-pentyl)-4-phenyl-4,5-dihydro-(1H)-pyrazol-1-yl]methanone
N-Endo-[(1R,2S,4R)-1,7,7-trimethylbicyclo[2.2. 1 ]hept-2-yl]-3-(n-pentyl)-4-phenyl-4,5-dihydro-(1H)-pyrazole-1-carboxamide (diastereomer A)
N-Endo-[(1R,2S,4R)-1,7,7-trimethylbicyclo[2.2.1]hept-2-yl]-3-(n-pentyl)-4-phenyl-4,5-dihydro-(1H)-pyrazole-1-carboxamide (diastereomer B)
N-Endo-[(1R,2S,4R)-1,7,7-trimethylbicyclo[2.2.1]hept-2-yl]-3-(n-butyl)-4-(3-fluorophenyl)-4,5-dihydro-(1H)-pyrazole-1-carboxamide (diastereomer A)
N-Endo-[(1R,2S,4R)-1,7,7-trimethylbicyclo[2.2.1]hept-2-yl]-3-(n-butyl)-4-(3-fluorophenyl)-4,5-dihydro-(1H)-pyrazole-1-carboxamide (diastereomer B)
N-Endo-[(1R,2S,4R)-1,7,7-trimethylbicyclo[2.2.1]hept-2-yl]-3-(n-butyl)-4-(4-chlorophenyl)-4,5-dihydro-(1H)-pyrazole-1-carboxamide (diastereomer A)
N-Endo-[(1R,2S,4R)-1,7,7-trimethylbicyclo[2.2.1]hept-2-yl]-3-(n-butyl)-4-(4-chlorophenyl)-4,5-dihydro-(1H)-pyrazole-1-carboxamide (diastereomer B)
N-(1,2,2,6,6-pentamethylpiperidin-4-yl)-3-(n-buty4-(2-fluorophenyl)-4,5-dihydro-(1H)-pyrazole-1-carboxamide
N-(4-methoxyphenyl)-3-(n-pentyl)-4-phenyl-4,5-dihydro-(1H)-pyrazole-1-carboxamide
N-(4-methoxyphenyl)-3-(n-pentyl)-4-phenyl-4,5-dihydro-(1H)-pyrazole-1-carboxamide
N-(phenethyl)-3-(n-pentyl)-4-phenyl-4,5-dihydro-(1H )-pyrazole-1-carboxamide
N-(2-phenyl-trans-cyclopropyl)-3-(n-pentyl)-4-phenyl-4,5-dihydro-(1H)-pyrazole-1-carboxamide
N-(1-naphthalen-1-yl-ethyl)-3-(n-pentyl)-4-phenyl-4,5-dihydro-(1H)-pyrazole-1-carboxamide
N-[2-(trifluoromethyl)phenyl)-3-(n-pentyl)-4-phenyl-4,5-dihydro-(1H)-pyrazole-1-carboxamide
N-cycloheptyl-3-(n-pentyl)-4-phenyl-4,5-dihydro-(1H)-pyrazole-1-carboxamide
N-cyclooctyl-3-(n-pentyl)-4-phenyl-4,5-dihydro-(1H)-pyrazole-1-carboxamide
N-(1,2,3,4-tetrahydronaphthalen-1-yl)-3-(n-pentyl)-4-phenyl-4,5-dihydro-(1H)-pyrazole-1-carboxamide
N-[2,2-(diphenyl)ethyl]-3-(n-pentyl)-4-phenyl-4,5-dihydro-(1H)-pyrazole-1-carboxamide
(3-Pentyl-4-phenyl-4,5-dihydropyrazol-1-yl)-[4-(2-pyrimidinyl)piperazin-1-yl]methanone
N-[2-(4-fluorophenyl)ethyl]-3-(n-pentyl)-4-phenyl-4,5-dihydro-(1H)-pyrazole-1-carboxamide
(3-Pentyl-4-phenyl-4,5-dihydropyrazol-1-yl)[azepan-1-yl]methanone
N-(quinolin-3-yl)-3-(n-pentyl)-4-phenyl-4,5-dihydro-(1H)-pyrazole-1-carboxamide
N-[1-(ethyl)propyl]-3-(n-pentyl)-4-phenyl-4,5-dihydro-(1H)-pyrazole-1-carboxamide
N-(2,2,2-trifluoroethyl)-3-(n-pentyl)-4-phenyl-4, 5-dihydro-(1H)-pyrazole-1-carboxamide
N-(pyridin-3-ylmethyl)-3-(n-pentyl)-4-phenyl-4,5-dihydro-(1H)-pyrazole-1-carboxamide
N-(2-indanyl)-3-(n-pentyl)-4-phenyl-4,5-dihydro-(1H )-pyrazole-1-carboxamide
(3-Pentyl-4-phenyl-4,5-dihydropyrazol-1-yl(1,2,3,4-tetrahydroisoquinolin-2-yl)methanone
N-(Methyl), N-(naphthalen-1-ylmethyl)-3-(n-pentyl)-4-phenyl-4,5-dihydro-(1H)-pyrazole-1-carboxamide
N-(3,3-Diphenypropyl)-3-(n-pentyl)-4-phenyl-4,5-dihydro-(1H)-pyrazole-1-carboxamide
N-(napht-1-yl)-3-(n-pentyl)-4-phenyl-4,5-dihydro-(1H)-pyrazolecarbothiamide
N-[1-(ethyl)propyl]-3-(n-pentyl)-4-phenyl-4,5-dihydro-(1H)-pyrazolecarbothiamide
N-[pyridin-3-ylmethyl]-3-(n-pentyl)-4-phenyl-4,5-dihydro-(1H)-pyrazolecarbothiamide
N-[exo-bicyclo[2.2.1]hept-2-yl]-3-(n-pentyl)-4-phenyl-4,5-dihydro-(1H)-pyrazolecarbothiamide
1-(Naphthalen-1-ylsulfonyl)-3-(n-butyl)-4-(2-fluorophenyl)-4,5-dihydro-(1H)-pyrazole
1-(Naphthalen-2-ylsulfonyl)-3-(n-butyl)-4-(2-fluorophenyl)-4,5-dihydro-(1H)-pyrazole
N-[(1R,2S,5R)-rel-6,6-dimethylbicyclo[3.1.1]heptan-2-methyl]-3-(n-pentyl)-4-phenyl-4,5-dihydro-(1H)-pyrazole-1-sulfonamide

9. Compounds of the general formula (IV). 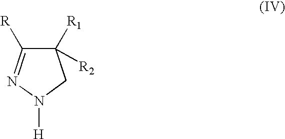
wherein R and R₁ have the same meanings as given in claim 1 and R₂ represents an phenyl group which may be substituted with 1-5 substituents Y2 which can be the same or different, selected from the group C₁₋₃-alkoxy, hydroxy, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethyl-sulfonyl, cyano, carbamoyl, sulfamoyl, ortho-halogen, meta-halogen, ortho-C₁₋₃-alkyl, meta-C₁₋₃-alkyl and acetyl, or R₂ represents a thienyl or pyridyl group, which groups may be substituted with one or two substituents Y, which Y group has the meaning as given in claim 1, such compounds being useful in the synthesis of compounds of the general formula (I).

10. Compounds of the general formula (III) 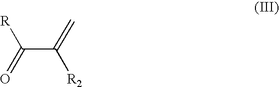
wherein:
R represent a phenyl group which is substituted with 1-3 substituents Y1 wherein Y1 represents halogen, CF₃, OCF₃ or OCH₃, or R represents a pyridyl or thienyl group, and R₂ represents a n-butyl, n-propyl, 1,1-dimethylpropyl, 1,1-dimethylbutyl, 3,3,3-trifluoropropyl, 4,4,4-trifluorobutyl or 1,1-dimethyl-3,3,3-trifluoropropyl group, or R represent a phenyl group and R₂ represents a 1,1-dimethylpropyl, 1,1-dimethylbutyl, 3,3,3-trifluoropropyl, 4,4,4-trifluorobutyl or 1,1-dimethyl-3,3,3-trifluoropropyl group, such compound being useful in the synthesis of compounds of the general formula (I).

11. Compounds of the general formula (VIII) 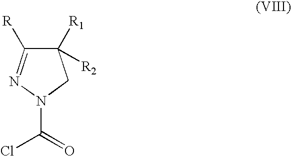
wherein R and R₂ have the same meanings as given in claim 1 and R₁ represents hydrogen, such compounds being useful in the synthesis of compounds of the general formula (I) wherein n=1.

12. A compound as claimed in any of the claims 1-8, or a pharmacologically acceptable salt, hydrate, solvate or complex thereof, for use as a medicament.

13. A medicament, characterized in that it contains a compound according to one of the claims 1-8, or a pharmacologically acceptable salt, hydrate, solvate or complex thereof.

14. A pharmaceutical composition comprising, in addition to a pharmaceutically acceptable carrier and/or at least one pharmaceutically acceptable auxiliary substance, a pharmacologically active amount of at least one compound of one of the claims 1-8, or a pharmacologically acceptable salt, hydrate, solvate or complex thereof, as an active ingredient.

15. A pharmaceutical composition as claimed in claim 14, for the prevention or the treatment of multiple sclerosis, traumatic brain injury, pain, appetite disorders, epilepsy, Alzheimer's disease, Tourette's syndrome, cerebral ischaemia or gastrointestinal disorders.

16. The pharmaceutical composition according to claim 14, further comprising: at least one additional therapeutic agent.

17. A method of preparing pharmaceutical compositions as claimed in claim 14, characterized in that a compound of one of the claims 1-8 is brought into a form suitable for administration.

18. A pharmaceutical composition made by mixing a compound of claim 1 and a pharmaceutically acceptable carrier and/or at least one pharmaceutically acceptable auxiliary substance.

19. Use of a compound as claimed in claims 1-8 for the preparation of a pharmaceutical composition for the treatment of multiple sclerosis, traumatic brain injury, pain, appetite disorders, epilepsy, Alzheimer's disease, Tourette's syndrome, cerebral ischaemia and gastrointestinal disorders.

20. A method of treating multiple sclerosis, traumatic brain injury, pain, appetite disorders, epilepsy, Alzheimer's disease, Tourette's syndrome, cerebral ischaemia or gastrointestinal disorders in a human or animal patient in need of such treating, wherein the method comprises administering to the patient a compound of formula (I) as claimed in claim 1 in an amount efficacious for the treating.

21. A method of modulating a cannabinoid CB₁ receptor, which comprises administering to a subject in need thereof, an effective amount of a compound according to claim 1.
